(19) United States
(12) Patent Application Publication
(10) Pub. No.: US 2026/0078152 A1
(43) Pub. Date: Mar. 19, 2026

(54) ENZYMATICALLY ACTIVE DOMAINS FROM ANTI-STAPHYLOCOCCAL PHAGE PROTEINS

(71) Applicant: Topaz Biosciences, Inc., Emeryville, CA (US)

(72) Inventors: Oliver LIU, San Francisco, CA (US); Teresa SHOCK, San Francisco, CA (US); Maritza MILLER, Oakland, CA (US); Jennifer SHOCK, San Francisco, CA (US)

(21) Appl. No.: 19/390,151

(22) Filed: Nov. 14, 2025

Related U.S. Application Data
(63) Continuation of application No. PCT/US2024/029487, filed on May 15, 2024.
(60) Provisional application No. 63/502,341, filed on May 15, 2023.

(51) Int. Cl. C07K 14/31; A61K 9/00; A61P 31/04; C12N 9/14

(52) U.S. Cl. CPC C07K 14/31; A61K 9/0014; A61P 31/04; C12N 9/14; C07K 2319/00

(57) ABSTRACT

The present disclosure relates to novel enzymatically active domains from Staphylococcus phage proteins. The disclosure also relates to cell wall hydrolases comprising these enzymatically active domains and uses thereof in the treatment of infection with Staphylococcus sp.

### CROSS-REFERENCE TO RELATED APPLICATIONS

This application is a continuation of International Application No. PCT/US2024/029487, filed May 15, 2024, which claims the benefit of priority to U.S. Provisional Application No. 63/502,341, filed May 15, 2023, the contents of each of which are incorporated by reference herein in their entireties.

### INCORPORATION OF THE SEQUENCE LISTING

The contents of the electronic sequence listing (TOPB_001_01US_SeqList_ST26.xml: Size: 161,688 bytes; and Date of Creation: Oct. 24, 2025) are herein incorporated by reference in their entirety.

### FIELD OF THE DISCLOSURE

The present disclosure relates to novel enzymatically active domains from anti-Staphylococcal phage proteins. The disclosure also relates to cell wall hydrolases comprising these enzymatically active domains.

### BACKGROUND

Staphylococcus is a genus of gram-positive bacteria in the family Staphylococcaceae from the order Bacillales. This genus includes at least 43 species, many of which do not cause disease and reside on the skin and mucous membranes of humans and other animals. However, some species of Staphylococcus, including Staphylococcus aureus, are pathogenic and can cause infections that are particularly dangerous to immunocompromised subjects. Staphylococcus aureus is a leading agent of sepsis. Within the skin microbiome, overgrowth of Staphylococcus aureus is strongly associated with moderate to severe cases of atopic dermatitis as well as acute radiation dermatitis.

While antibiotics may be used to treat bacterial infection, many Staphylococcus strains are antibiotic resistant, making them very difficult to manage. In addition, antibiotics or chemicals like benzoyl peroxide can negatively affect commensal bacterial populations, as well as pathogenic ones.

There is an ongoing, unmet need for new therapeutics active against Staphylococcus sp.

### BRIEF SUMMARY

The present disclosure provides recombinant proteins comprising enzymatically active domains disclosed herein. In one aspect, the present disclosure provides recombinant proteins comprising the sequence of any one of SEQ ID NO: 2 or 4-123, or a sequence having at least 70, 80, 90, 95, or 99% sequence identity to any one of SEQ ID NO: 2 or 4-123.

In another aspect, the present disclosure provides a chimeric cell wall hydrolase (CWH) comprising: a) an enzymatically active domain (EAD) having the sequence of CHAP2 (SEQ ID NO: 2) or M23-S1 (SEQ ID NO: 4), or a sequence having at least 70, 80, 90, 95, or 99% sequence identity to the sequence of CHAP2 (SEQ ID NO: 2) or M23-SI (SEQ ID NO: 4); and b) a cell wall binding domain (CBD) from ALE-1 (SEQ ID NO: 127), LysA72 (SEQ ID NO: 125), LysH5 (SEQ ID NO: 124), LysSA97 (SEQ ID NO: 128), LysPALS1 (SEQ ID NO: 129), or PlySs2 (SEQ ID NO: 126), or a CBD having at least 70, 80, 90, 95, or 99% sequence identity to a CBD from ALE-1 (SEQ ID NO: 127), LysA72 (SEQ ID NO: 125), LysH5 (SEQ ID NO: 124), LysSA97 (SEQ ID NO: 128), LysPALS1 (SEQ ID NO: 129), or PlySs2 (SEQ ID NO: 126).

In another aspect, the present disclosure provides a chimeric cell wall hydrolase (CWH) comprising: a) an enzymatically active domain (EAD) from Lysostaphin (SEQ ID NO: 152) or ALE-1 (SEQ ID NO: 153), or a sequence having at least 70, 80, 90, 95, or 99% sequence identity to an EAD from Lysostaphin (SEQ ID NO: 152) or ALE-1 (SEQ ID NO: 153); and b) a cell wall binding domain (CBD) from LysH5 (SEQ ID NO: 124), or a CBD having at least 70, 80, 90, 95, or 99% sequence identity to a CBD from LysH5 (SEQ ID NO: 124).

In another aspect, the present disclosure provides a chimeric cell wall hydrolase (CWH) comprising: a) an enzymatically active domain (EAD) having the sequence of M23-S66 (SEQ ID NO: 69), or a sequence having at least 70, 80, 90, 95, or 99% sequence identity to the sequence of M23-S66 (SEQ ID NO: 69); and b) a cell wall binding domain (CBD) from LysH5 (SEQ ID NO: 124), or a CBD having at least 70, 80, 90, 95, or 99% sequence identity to a CBD from LysH5 (SEQ ID NO: 124).

In another aspect, the present disclosure provides a composition comprising a first and a second recombinant protein, wherein each recombinant protein comprises: an enzymatically active domain (EAD) having the sequence of any one of SEQ ID NO: 2 or 4-123, or a sequence having at least 70, 80, 90, 95, or 99% sequence identity to any one of SEQ ID NO: 2 or 4-123.

In another aspect, the present disclosure provides a topical formulation comprising a recombinant protein, chimeric cell wall hydrolase, or combination composition of the disclosure.

In another aspect, the present disclosure provides methods of treating conditions associated with Staphylococcus comprising administering a composition or formulation disclosed herein.

### BRIEF DESCRIPTION OF THE SEVERAL VIEWS OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1A shows the domain architecture of ANT44936.1 as determined via SMART.

FIG. 1B shows the domain architecture of YP_008058813 as determined via SMART.

FIG. 2A-2F show turbidity reduction assay results for chimeric cell wall hydrolases comprised of the CHAP2 EAD fused to a panel of CBD domains from staphylococcal cell wall hydrolases, as compared to a Twort EAD+PlySs2 CBD chimera. Results are shown for the following chimeras: CHAP2 EAD+PALS1 CBD (FIG. 2A): CHAP2 EAD+ALE-1 CBD (FIG. 2B): CHAP2 EAD+LysSA97 CBD (FIG. 2C): CHAP2 EAD+PlySs2 CBD (FIG. 2D); CHAP2 EAD+LysH5 CBD (FIG. 2E); and Twort EAD+PlySs2 CBD (FIG. 2F).

FIG. 3 shows relative activity of CHAP2 chimeric enzymes against S. aureus and S. epidermidis as calculated from turbidity reduction assays, and as compared to the Twort EAD+PlySs2 CBD control.

FIG. 4 shows Minimal Inhibitory Concentration (MIC) assay results against Staphylococcus aureus and Staphylococcus epidermidis for chimeric cell wall hydrolases comprised of the CHAP2 EAD fused to a panel of CBDs from staphylococcal cell wall hydrolases, and as compared to a Twort EAD+PlySs2 CBD chimera control. Wells outlined in the figure showed no visible bacterial growth.

FIG. 5A-5E show turbidity reduction assay results for chimeric cell wall hydrolases comprised of the M23-SI EAD fused to a panel of CBD domains from staphylococcal cell wall hydrolases, as compared to a Twort EAD+PlySs2 CBD chimera. Results are shown for the following chimeras: M23-S1 EAD+ALE-1 CBD (FIG. 5A): M23-S1 EAD+LysA72 CBD (FIG. 5B): M23-SI EAD+PlySs2 CBD (FIG. 5C): M23-S1 EAD+LysH5 CBD (FIG. 5D); and Twort EAD+PlySs2 CBD (FIG. 5E).

FIG. 6 shows relative activity of M23-SI chimeric enzymes against S. aureus and S. epidermidis as calculated from turbidity reduction assays.

FIG. 7A-7E show thermostability assay results for chimeric cell wall hydrolases comprised of the M23-SI EAD fused to a panel of CBD domains from staphylococcal cell wall hydrolases, as compared to a Twort EAD+PlySs2 CBD chimera. Results are shown for the following chimeras: M23-S1 EAD+ALE-1 CBD (FIG. 7A): M23-SI EAD+LysA72 CBD (FIG. 7B): M23-S1 EAD+PlySs2 CBD (FIG. 7C): M23-S1 EAD+LysH5 CBD (FIG. 7D); and Twort EAD+PlySs2 CBD (FIG. 7E).

FIG. 8 shows Minimal Inhibitory Concentration (MIC) assay results against Staphylococcus aureus and Staphylococcus epidermidis for M23-S1-LysH5 and M23-S1-ALE-1 chimeric cell wall hydrolases, as compared to a Twort EAD+PlySs2 CBD chimera control. Wells outlined in the figure showed no visible bacterial growth.

FIG. 9 shows the results of M23-S1 EAD+LysH5 CBD in a turbidity reduction assay against a variety of Staphylococcus sp.

FIG. 10A-10B show turbidity reduction assay results for SA. 100 vs. M23-SI EAD+LysH5 CBD. FIG. 10A shows the turbidity reduction assay results, while FIG. 10B shows relative activity as calculated from the turbidity reduction assay.

FIG. 11 shows the results of quantitative killing assays for SA. 100 vs. M23-S1 EAD+LysH5 CBD.

FIG. 12 shows the results of an assay testing the effect of an M23-S1 EAD+LysH5 CBD chimera compared to SA. 100 or PBS alone in a 3D skin model of the skin microbiome. Error bars represent the standard error of the mean of 3 biological replicates.

FIG. 13 shows the results of a checkerboard assay used to measure synergistic activity against Staphylococcus aureus by combinations of M23-SI EAD+LysH5 CBD and CHAP2 EAD+LysH5 CBD chimeric CWHs. The numbers in each well are the calculated Fractional Inhibitory Concentration. The bottom row shows results for the M23-SI EAD+LysH5 CBD chimeric CWH alone—the well with the dotted lines around it corresponds to the minimum inhibitory concentration for this chimeric CWH. The rightmost column shows results for the CHAP2 EAD+LysH5 CBD chimeric CWH alone—the well with the dotted lines around it corresponds to the minimum inhibitory concentration for this chimeric CWH. The well with the solid black line around it corresponds to a combination with observed synergistic results.

FIG. 14 shows a phylogenetic tree of 120 M23 domains found in Staphylococcal phage tail proteins. The 120 non-redundant sequences are divided into 11 families and 36 groups, as shown in the tree.

FIG. 15A-15B show the results of turbidity reduction assays against S. aureus, testing representative chimeric proteins comprising the LysH5 CBD in combination with an M23 EAD from groups 1-19 (FIG. 15A) and groups 20-36 (FIG. 15B). Chimeras that were active in this assay are marked with *.

FIG. 16 shows the M23 EADs that demonstrated S. aureus antimicrobial activity when fused to the LysH5 CBD, i.e., in the results shown in FIG. 15A-15B.

FIG. 17A-17B show the results of assays conducted with purified M23-S66 EAD+LysH5 CBD chimeric protein. FIG. 17A shows the results of turbidity reduction assays testing for microbial activity against Staphylococcus aureus, Staphylococcus epidermidis, Cutibacterium acnes, and Corynebacterium striatum, while FIG. 17B shows the results of thermostability assays.

FIG. 18A-18B show motif logos for M23 EAD sequence motif 1 (FIG. 18A) and M23 EAD sequence motif 2 (FIG. 18B).

FIG. 19A-19D show the results of turbidity reduction assays for wild type lysostaphin compared to a Lss-EAD+LysH5-CBD chimera against S. aureus (FIG. 19A) and S. epidermidis (FIG. 19B); and for wild type ALE-1 compared to an ALE-1-EAD+LysH5-CBD chimera against S. aureus (FIG. 19C) and S. epidermidis (FIG. 19D).

FIG. 20 shows the results of turbidity reduction assays against S. aureus and S. epidermidis for a LytM-EAD+LysH5-CBD chimera.

FIG. 21A-21B show the results of quantitative killing assays against S. aureus for M23-S1 EAD+LysH5 CBD chimeric protein in two hydrogel formulations: Formulation #1 (FIG. 21A) and Formulation #2 (FIG. 21B).

### DETAILED DESCRIPTION

All publications, patents and patent applications, including any drawings and appendices, are herein incorporated by reference to the same extent as if each individual publication or patent application was specifically and individually indicated to be incorporated by reference.

The following description includes information that may be useful in understanding the present disclosure. It is not an admission that any of the information provided herein is prior art, or that any publication specifically or implicitly referenced is prior art.

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “cell wall hydrolase” or “CWH” refers to bacterial cell wall hydrolases, which are enzymes that degrade peptidoglycan in bacterial cell walls by cleaving bonds in the peptidoglycan chain and side-chain branches. Cell wall hydrolases may have different domain architectures. CWHs comprise an “enzymatically active domain” or “EAD,” which is a domain responsible for degrading peptidoglycan. In some embodiments, the EAD has glycosidase, amidase, and/or peptidase enzymatic activity. In some embodiments, CWHs comprise a “cell wall binding domain” or “CBD”, which is a domain that binds to a bacterial cell wall. In some embodiments, CWHs of the disclosure are recombinant proteins. In some embodiments, CWHs of the disclosure are chimeric proteins.

A “native” protein is used to indicate a protein that occurs in nature and has not been artificially modified or recombined.

The term “recombinant” is used herein to describe nucleic acids, proteins, vectors, and host cells which do not occur in nature or, in the context of nucleic acids, are in an arrangement not found in nature. A “recombinant protein” therefore refers to a protein which does not occur in nature. In some embodiments, a recombinant protein, as used herein, refers to a chimeric protein. In some embodiments, recombinant protein refers to the expression product of any of the presently disclosed EAD or CBD sequences alone, or within a protein that does not occur in nature. For example, the present disclosure envisions recombinant EAD or CBD sequences of the disclosure fused to any protein tags, such as 6× His.

As used herein, “heterologous” refers to any genetic material that is artificially introduced into a non-native context. E.g., a heterologous domain refers to a domain, such as an EAD or CBD, that is artificially introduced into a recombinant protein sequence, wherein the resulting recombinant protein sequence is non-native.

As used herein, a “chimeric protein” is any recombinant protein comprising two or more heterologous domains, e.g., EADs and/or CBDs.

As used herein, a “domain” of a protein is a functional and/or structural subunit in a protein. In some embodiments, they are responsible for a particular function or interaction, contributing to the overall role of a protein. Protein domains are fundamental units of protein structure, folding, function, evolution and design. See, e.g., Wang et al., “Protein domain identification methods and online resources,” Comput Struct Biotechnol J 2021:19:1145-1153, incorporated by reference herein.

As used herein, a “chimeric cell wall hydrolase” or “chimeric CWH” is a chimeric protein that acts as a cell wall hydrolase and comprises at least one heterologous domain, e.g., a heterologous EAD or CBD, compared to a native CWH sequence. A chimeric CWH also refers herein to a recombinant protein comprising two or more heterologous CWH domains, e.g., an EAD and a CBD. A chimeric CWH also refers to a CWH comprising at least two CWH domains that are not found together in nature.

As used herein, “activity” refers to the ability of a chimeric protein to inhibit the growth of and/or lyse a cell from a Staphylococcus species. The term “active against”, as used herein with reference to a target species of Staphylococcus, refers to a protein, e.g., a CWH, comprising an EAD of the disclosure that is able to inhibit the growth of and/or lyse cells belonging to that target species of Staphylococcus. Activity can be calculated in different ways, depending on the assay performed. In some embodiments, level of activity is indicated based on minimum inhibitory concentration (“MIC”), e.g., the minimum concentration of the protein required to prevent growth of the target Staphylococcus species in an MIC assay. In some embodiments, level of activity is indicated based on turbidity reduction, e.g., with activity calculated as −αOD₆₀₀/min/(mg of enzyme). In some embodiments, activity is indicated based on the decrease in viable bacterial cells in a culture after a period of incubation (e.g., 2 hours) with a protein.

In the context of anti-Staphylococcus activity, the terms “selective” and “selectivity” as used herein refer to the property of showing higher activity toward one target species of Staphylococcus in comparison to a second species of Staphylococcus. Selectivity can be calculated by comparing the inverse of the MIC of a chimeric protein toward a first species to the MIC of the protein toward a second species. In some embodiments, selectivity is determined based on relative activity in a turbidity reduction assay.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs. Unless noted otherwise, the term “sequence identity” in the claims refers to sequence identity as calculated by MUSCLE (www.ebi.ac.uk/Tools/msa/muscle/) using default parameters.

### Overview

The present disclosure provides novel enzymatically active domains (EADs) from Staphylococcus phage proteins. The disclosure also provides chimeric proteins, e.g., chimeric cell wall hydrolases, comprising these EADs. The disclosure also provides novel chimeric proteins comprising combinations of EADs and CBDs from different native proteins. Also provided herein are compositions comprising the EADs or chimeric proteins, and uses thereof in targeting Staphylococcus sp.

The present disclosure is related, in part, to the growing appreciation for the critical role that the skin microbiome plays in skin health and skin function. Among other benefits, a healthy microbiome helps to prevent colonization by pathogenic microbes, train the immune system and prevent inflammation, reinforce the skin barrier, and promote wound healing. Dysbiosis of the skin microbiome can cause and/or exacerbate skin diseases like atopic dermatitis and acne vulgaris. For example, overgrowth of Staphylococcus aureus is strongly associated with moderate to severe cases of atopic dermatitis as well as acute radiation dermatitis.

Current therapeutic tools do not allow for precision treatment of skin diseases. Common approaches like topical/oral antibiotics or chemicals like benzoyl peroxide are used to target causal bacteria but also negatively affect the global skin microbiome. In other cases, long term steroid use can lead to unwanted and severe side effects.

The present disclosure provides compositions and treatments for Staphylococcus infection based on the identification of novel EADs from cell wall hydrolases (CWHs) and based on the identification of novel combinations of EADs and CBDs from CWHs. CWHs are enzymes that degrade bacterial peptidoglycan by cleaving bonds in the peptidoglycan chain and side-chain branches. Degradation of peptidoglycan cell walls by CWHs can result in rapid lysis of a bacterial cell due to an inability to resist internal turgor pressure.

As demonstrated in the Examples herein, the present disclosure provides highly effective chimeric CWHs comprising novel EADs, and/or novel combinations of EADs and CBDs. These chimeric CWHs have high lytic activity and/or Staphylococcus species-specificity. In some embodiments, CWHs herein bind very specific epitopes in target cell walls. In some embodiments, CWHs herein have lytic activity down to a single species or group of related species. Because of these properties, in some embodiments, the CWHs herein act as high-specificity skin microbiome modulators. For example, in the case of atopic dermatitis or acute radiation dermatitis, in some embodiments, CWHs herein are able to specifically kill Staphylococcus aureus, while exhibiting significantly less activity against healthy, commensal bacteria.

Novel Enzymatically Active Domains (EADs) from Staphylococcus Phage Proteins

The present disclosure is based in part on the inventors discovery of novel, previously uncharacterized, and highly active EADs isolated from Staphylococcus phage proteins. These EADs are surprising in that they are derived from proteins having noncanonical CWH architecture and yet the EADs have remarkably high anti-Staphylococcus activity against target Staphylococcus species.

In some embodiments, the EAD is derived from Staphylococcus phage vB_SscM-2. In some embodiments, the EAD is derived from Genbank Accession No. ANT44936.1. In some embodiments, the EAD is a CHAP domain. In some embodiments, the EAD consists of SEQ ID NO: 2, herein referred to as the CHAP2 domain. In some embodiments, the EAD comprises the sequence of SEQ ID NO: 2. In some embodiments, the EAD has at least 80, 81, 82, 83, 84, 85, 86, 87, 88, 89, 90, 91, 92, 93, 94, 95, 96, 97, 98, or 99% identity with the sequence of SEQ ID NO: 2. In some embodiments, the EAD differs from the sequence of SEQ ID NO: 2 by 1, 2, 3, 4, 5, 6, 7, 8, 9, 10, 11, 12, 13, 14, or 15 amino acids.

The sequence of SEQ ID NO: 2 is as follows:

DWDNYAGWQCFDTANYHAYFAMGMSLAGEGAKDIPYKNDFTGKADIY
NNTPEFLAQPGDIVVWTSPTFGGGYGHVASVISATLNTITVIEQN.

In some embodiments, the EAD is derived from Staphylococcus phage StauST398-2. In some embodiments, the EAD is derived from Genbank Accession No. YP_008058813. In some embodiments, the EAD is an M23 domain. In some embodiments, the EAD consists of SEQ ID NO: 4, herein referred to as the “M23-S1” or the “SI” EAD, domain, or sequence. In some embodiments, the EAD comprises the sequence of SEQ ID NO: 4. In some embodiments, the EAD has at least 80, 81, 82, 83, 84, 85, 86, 87, 88, 89, 90, 91, 92, 93, 94, 95, 96, 97, 98, or 99% identity with the sequence of SEQ ID NO: 4. In some embodiments, the EAD differs from the sequence of SEQ ID NO: 4 by 1, 2, 3, 4, 5, 6, 7, 8, 9, 10, 11, 12, 13, 14, or 15 amino acids.

The sequence of SEQ ID NO: 4 is as follows:

GRHYGIDFGMPSGTNVYAVKGGIADKVWTDYGGGNSIQIKTGANEWN
WYMHLSKQLARQGQRIKAGQLIGKSGATGNFVRGAHLHFQLMQGSHP
GNDTAKDP.

In some embodiments, the EAD consists of SEQ ID NO: 69, herein referred to as the “M23-S66” or the “S66” EAD, domain, or sequence. In some embodiments, the EAD comprises the sequence of SEQ ID NO: 69. In some embodiments, the EAD has at least 80, 81, 82, 83, 84, 85, 86, 87, 88, 89, 90, 91, 92, 93, 94, 95, 96, 97, 98, or 99% identity with the sequence of SEQ ID NO: 69. In some embodiments, the EAD differs from the sequence of SEQ ID NO: 69 by 1, 2, 3, 4, 5, 6, 7, 8, 9, 10, 11, 12, 13, 14, or 15 amino acids.

The sequence of SEQ ID NO: 69 is as follows:

GRHYGMDFGMPAGTSVYAVKGGTVDKLWYDYGGGNSIQIKTGPGEWN
WYMHLSKQLVKLGEQFKTGQLIAKSGETGAYCKGAHLHFQLMRGDHP
GNDTAIDP.

In some embodiments, the EAD consists of a sequence contained in Table 1. In some embodiments, the EAD comprises a sequence contained in Table 1.

TABLE 1
M23 EAD Sequences of the Disclosure.

| Sequence Name, | SEQ | |
|---|---|---|
| Group & Family | ID NO | Amino Acid Sequence |
| M23-S1_G26_FamH | 4 | GRHYGIDFGMPSGTNVYAVKGGIADKVWTDYGGGN |
| | | SIQIKTGANEWNWYMHLSKQLARQGQRIKAGQLIG |
| | | KSGATGNFVRGAHLHFQLMQGSHPGNDTAKDP |
| M23-S2_G14_FamD | 5 | PFHEGVDFPFVYQEVRTPMGGRLTRMPFMSGGYGN |
| | | YVKITSGVIDMLFAHLKNFSKSPPSGTMVKPGDVV |
| | | GLTGNTGFSTGPHLHFEMRRNGRHFDP |
| M23-S3_G26_FamH | 6 | GRHYGIDFQMPTGTNIYAVKGGIADKVWTDYGGGN |
| | | SIQIKTGANEWNWYMHLSKQLARQGQRIKAGQLIG |
| | | KSGATGNFVRGAHLHFQLMRGSHPGNDTAVDP |
| M23-S4_G22_FamF | 7 | GVHHGVDYDTPTGTPIRTPMGGRVRSWYDNYGGGK |
| | | AITVQKGRTFLWFMHLSEQLRRTGEQIKAGQLIGK |
| | | SGNTGSMTNYRHLHFQVNQGGESNRYSTDP |
| M23-S5_G24_FamH | 8 | GRHYGMDFGMPAGTKVYAVKGGTVDNVWYDYGGGN |
| | | SIQIKTGPGEWNWYMHLSKQLVRLGEHIRTGQLIA |
| | | ESGATGAFCKGAHLHFQLMRGDHPGNDTAIDP |
| M23-S6_G17_FamE | 9 | GHHYGIDYATPYGTLITAPTSGTVSRQYNQYGGLI |
| | | ARLVSGIYAQYFLHLSEVLKTGRVEQGEPIARTGN |
| | | SGQWTTGPHLHYQVESPFGAELTNRNTINP |
| M23-S7_G18_FamE | 10 | GHHYGIDFGAPYGTTINATNSGQLGELHNFGGGLV |
| | | ARLLTGQFTLFFMHLSKILKHGKVQAGEPIAKTGN |
| | | SGNWTTGPHLHFQVEKGRHNDITNQNTVNP |
| M23-S8_G1_FamA | 11 | RAHHGLDINYKYDKVYSTLSGIATGSSGWNGGFGQ |
| | | NMWIRAKGGLEAIYGHLHKLAFHGKKRVKPGTYLG |
| | | ISGGDPGRDGQNAGSSTGPHLHYEMRRNGVPFDP |
| M23-S9_G36_FamK | 12 | GVHHGRDITSATINGSPIKAARSGIVTFKGWTGGG |
| | | NTLSIFDGKNTYTYMHMKNPARVVKGQRVKAGQIV |
| | | GNVGTTYDRRLGGFSTGPHLHVQANLGKTPSGTFM |
| | | N |
| M23-S10_G28_FamH | 13 | GKHYGMDFGMLPGTNVYAVAGGKASRVWHDYGGGN |
| | | SIEVDLGGGLTNWYMHLQKQLVKQGQRIKAGDLIA |
| | | KSGNTGAFTAGTGHLHFQLNKNGVPKDP |
| M23-S11_G22_FamF | 14 | GVHHGVDYDTPVGTPIRTPMAGRVRSWYDNYGGGN |
| | | AITVSKGKTFLWFMHLSKQLKKTGEQVKAGQLIGK |
| | | SGNTGSMTNYRHLHFQVNQGGESNSNSVEP |
| M23-S12_G14_FamD | 15 | PFHEGVDFPFVYQEVRTPMGGRLTRMPFMSGGYGN |
| | | YVKITSGVIDMLFAHLKNFSKSPPSGTMVKPGDVV |
| | | GLTGNTGFSTGPHLHFEMRRNGRHFNP |
| M23-S13_G13_FamD | 16 | PFHEGLDFDYIYEPVPSTINGRAQVMPFHNGGYGK |
| | | WVKIVKGTLEVIYAHLSKYKVKTGQQVRVGQTVGI |
| | | SGNTGFSTGPHLHYEMRWNGRHRDP |
| M23-S14_G13_FamD | 17 | PFHEGLDFDYIYEPVPSTINGRAQVMPVHNGGYGK |
| | | WVKIVKGALEVIYAHLSKYKVKTGQQVRVGQTVGI |
| | | SGNTGFSTGPHLHYEMRWNGRHRDP |
| M23-S15_G6_FamA | 18 | RAHHGLDIGYPYGTKVFSTTGGTATASKGWNGGFG |
| | | NMVSVKSGTMETIYGHLSKHAFSGSKKVKPGDLLG |
| | | LSGGDPSKQGREAGSSTGAHLHYEMRWGGVPKDP |
| M23-S16_G23_FamG | 19 | GKHYGIDFGMPTGTKIRALTAGKISQAGPVAGGGG |
| | | NQITLDEPGGKWFQWYMHMSKVIAKKGQRVNAGDV |
| | | IGLSGNTGNSTTPHLHIQRMKGYPSNETAINP |
| M23-S17_G26_FamH | 20 | GKHYGMDFGMPTGTPIYAVKGGVADKVWTDYGGGN |
| | | SVQIKTGANEWNWYMHLSKQIAKQGQKIRAGQLIG |
| | | KSGATGNFVRGAHLHFQLMRGSHAGNDTAVNP |
| M23-S18_G8_FamB | 21 | AHPGIDLPYHYEKVQTPLGGTIKTGEMPGGFGHYL |
| | | RVMAKPYDAYFGHLSKWLVKDGQHVSPSDAIAISG |
| | | NTGASTGPHLHFEMNKHGFGANTGHSIDP |
| M23-S19_G14_FamD | 22 | PFHEGVDFPFVYQEVRTPMGGRLTRMPFMSGGYGN |
| | | YVKITSGVIDMLFAHLKNFSKSPPSGTMVKPGDVV |
| | | GLTGNTGFSTGPHLHFEMRRNGQHFDP |
| M23-S20_G34_FamJ | 23 | TPHYGVDYASSVGSPIKASRGGIVLSSGWSNYGGG |
| | | NQVVIYNPKLNKTFTYMHMLSDLRVKKGQQVVAGQ |
| | | MIGRMGNTGNSTGPHLHFQVNEGKGFTAAGT |
| M23-S21_G32_FamI | 24 | TGHAGIDYAASVGTKIPSPLDGTVIKSWQSPWGGG |
| | | NETQVYDGSKYTHIFMHQSKRGVSTGDKVHQGQII |
| | | GLTGNTGNSTGPHLHWQVNKGKGFLNNHPDSINP |
| M23-S22_G22_FamF | 25 | GVHHGIDYDTPVGTPIRTPMGGRVRSWYDNYGGGK |
| | | AITVQQGKTFLWFMHLSQQLRKTGEQIKAGQLIGK |
| | | SGNTGSMTNYRHLHFQVNQGGEANRYSV |
| M23-S23_G23_FamG | 26 | GRHYGVDFGMPTGTKIKALTDGKISQAGAVAGGGG |
| | | NQITLDEPGGKWYQWYMHMSKIIAKKGQKVSAGDV |
| | | IGLSGNTGNSTTPHLHIQRMKGYPSNETAVDP |
| M23-S24_G4_FamA | 27 | YPHMGVDLNYVYDKLYSTHSGIATGKTGYNGGFGN |
| | | HMSIKSGIYEIIYGHMSKLAWTGSKRVHPGSYLGV |
| | | SGNTGMSSGPHLHYEMRKNGVPINP |
| M23-S25_G23_FamG | 28 | SRHYGVDFGMPTGTKIKALTDGKISQAGAVAGGGG |
| | | NQITLDEPGGKWYQWYMHMSKIIAKKGQKVSAGDV |
| | | IGLSGSTGNSTTPHLHIQRMKGYPSNETAVNP |
| M23-S26_G14_FamD | 29 | PFHEGVDFPFVYQTVRTPMGGRLTRMPFMSGGYGN |
| | | YVKITSGAIDMLFAHLKDFSKSPPSGSTVKPGDVV |
| | | GLTGNTGFSTGPHLHFEMRRNGRHFDP |
| M23-S27_G23_FamG | 30 | GRHYGIDFGMPTGTKIKALTDGTVTQAGAVAGGGG |
| | | NQVTLKEPGGKWYQWYMHMSKILTKKGAKVKAGDL |
| | | LGLSGNTGNSTTPHLHIQRMKGYPSNETAVNP |
| M23-S28_G30_FamI | 31 | GGHAGIDYAVPGGTKIPSPIDGEVIQRWFSPFGGG |
| | | NETQVYDGSKYTHIFMHQSKQIAKKGQRISQGDII |
| | | GLVGNTGNSFGDHLHWQVNKGKGFRNNHPDSINP |
| M23-S29_G4_FamA | 32 | YPHKGVDLNYVYDRLYSTHAGTATGSMGYNGGFGN |
| | | HMRIRSGIYEIIYGHMSKLNWTGSKKVRPGSFLGI |
| | | SGNTGMSSGPHLHYEMRKNGRAIDP |
| M23-S30_G9_FamB | 33 | AHPGIDLPYHYEKVQSTTSGIARTKDTGNVGFGHH |
| | | IVVEGKPYDVIYGHLSKWLVKNGEHVHPGTVLGIS |
| | | GSTGSSTGPHLHYEMNKHGFGSMTGHSIDP |
| M23-S31_G2_FamA | 34 | RPHFGLDINYKHDKVYSTMSGTAKTFNGWSGGFGR |
| | | HVEITNGNLKSIYGHLHKLAFNGTKKVRPGTFLGI |
| | | SGGDPREDGQNAGSSTGLHLHYEMQRNGRAFDP |
| M23-S32_G13_FamD | 35 | PFHEGLDFDYIYEPVPSTINGRAQVMPFHNGGYGK |
| | | WVKIVKGALEVIYAHLSKYKVKTGQQVRVGQTVGI |
| | | SGNTGFSTGPHLHYEMRWNGRHRDP |
| M23-S33_G14_FamD | 36 | PFHEGVDFPFVYQTVRTPMGGRLTRMPFMDGGYGN |
| | | YVKITSGAIDMLFAHLKNFSKSPPSGSTVKPGDVV |
| | | GLTGNTGFSTGPHLHFEMRRNGRHFDP |
| M23-S34_G30_FamI | 37 | GGHAGIDYAVPGGTKIPSPIDGEVIQRWFSPYGGG |
| | | NETQVYDGSKYTHIFMHQSKQIAKKGQRISQGDII |
| | | GLVGNTGNSFGDHLHWQVNKGRGFRNNHPDSINP |
| M23-S35_G22_FamF | 38 | GVHHGVDYDTPVGTPIRTPMGGRVRSWYDNYGGGK |
| | | AITVQKGRTFLWFMHLSEQLRRTGEQIKAGQLIGK |
| | | SGNTGSMTNYRHLHFQVNQGGEANRF |
| M23-S36_G4_FamA | 39 | YPHMGVDLNYVYDKLYSTHGGVATGKTGYNGGFGN |
| | | SMWIKSGIYEIIYGHMSKLAWTGAKKVHPGSYLGI |
| | | SGNTGMSSGPHLHYEMRKNGVPIDP |
| M23-S37_G22_FamF | 40 | GVHHGVDYDMPVGTPVRTPMAGRVRSWYDNYGGGN |
| | | AITVSKGKTFLWFMHLSKQLKKTGEQVKAGQLIGK |
| | | SGNTGSMTNYRHLHFQVNQGGESNSNSVEP |
| M23-S38_G18_FamE | 41 | GAHFGIDYGAPYGTTINATNDGNVKAIHNLGGGLV |
| | | ARLLTGQFTLFFMHLSKILKQGKIKAGEPMAKTGN |
| | | SGQWTTGPHVHFQVERGRHDDITNRGTVNP |
| M23-S39_G23_FamG | 42 | GRHYGMDFAMPTGTSIKALTAGKISQAGPVAGGGG |
| | | NQITLDEPGGKWFQWYMHMSKIIAKKGQRVNAGDV |
| | | IGLSGNTGNSTTPHLHIQRMKGYPSNETAVNP |
| M23-S40_G11_FamC | 43 | AHPGIDLPYHHEPVQSTVSGKAYRKYMPNGFGNYV |
| | | LVKSGNLEVFYGHLLKYLIKNGQSVRPGTKLAISG |
| | | GALSDPGHGASTGMHLHYEMHQNGKPINP |
| M23-S41_G4_FamA | 44 | YPHMGVDLNYVYDKLYSTHNGTATAKSGYNGGFGN |
| | | SMWIKSGIYEIIYGHMSKLAFNGSKKVHPGSYLGV |
| | | SGNTGMSSGPHLHYEMRKNGKPIDP |
| M23-S42_G27_FamH | 45 | GRHYGIDFGMPIGTPVKAVADGKVSKVWNDYGGGK |
| | | SIEVQVGKNLWNWYMHLSEQMAKQGQKVSAGDVIG |
| | | KSGDTGNFVNGAHLHFQLNKGDHSGNDTAINP |
| M23-S43_G7_FamA | 46 | RPHHGLDINYPYGTKVYSTVSGKATGSKGYNGGFG |
| | | NMMSVISGAIEVIYGHLSKLNWTGSKSVKPGTLLG |
| | | LSGGDPARQGAGAGSSTGPHLHYEMRWNGVAKDP |
| M23-S44_G36_FamK | 47 | GVHHGRDITSATINGSPIKAARSGIVTFKGWTGGG |
| | | NTLSIFDGKNTYTYMHMKNPARVVKGQRVKAGQIV |
| | | GNVGTTHDRRLGGFSTGPHLHVQVNLGKTPSGTFM |
| | | NT |
| M23-S45_G15_FamD | 48 | PFHEGVDFPFVYKTIRTPMGGKVARQSFMHGGYGN |
| | | WVKVISGAMEMIFAHLRDFSKTPPTGKTVKAGDVI |
| | | GLTGNTGFSTGPHLHFGIRKNGRDVDP |
| M23-S46_G26_FamH | 49 | GRHYGVDFGMPAGTNIYAVKGGIADRVWTDFGGGN |
| | | SIQIKTGANEWNWYMHLSKQIARQGQRIKAGQLIG |
| | | LSGATGNFVRGAHLHFQLMRGSHPGNDTAVDP |
| M23-S47_G26_FamH | 50 | GRHYGVDFGMDPGTNIYAVKGGIADKVWTDYGGGN |
| | | SIQIKTGANEWNWYMHLSKQLVRQGQRIKAGQLIG |
| | | KSGATGNFVRGAHLHFQLMQGSHPGNDTAKDP |
| M23-S48_G35_FamK | 51 | GAHYGLDITSANINNSAIRAAKSGVVTFKGWTGGG |
| | | NTISIFDGKNTYTYMHMIRPSKLKVGSTVQAGQHV |
| | | GNVGSTFGRGGRSTGPHLHVQVNKGRTPTGTFMD |
| M23-S49_G15_FamD | 52 | PFHEGVDFPFVYKTIRTPMGGKVARQSFMHGGYGN |
| | | WVKVISGAMEMIFAHLRDFSKTPPSGKTVKAGDVI |
| | | GLTGNTGFSTGPHLHFGIRKNGRDVDP |
| M23-S50_G26_FamH | 53 | GRHYGVDFGMPIGTNVYAVKGGKADKVWTDYGGGK |
| | | SVQIKTGQGEWNWYMHLSKQIAKQGQRIKPGQLIG |
| | | KSGDTGNFVRGAHLHFQLMKGSHAGNDTAKDP |
| M23-S51_G13_FamD | 54 | PFHEGLDFDYIYEPLPSTINGTAQVMPFMHGGYGN |
| | | WVKIVQGALEVIYAHLSKHKLKTGQKVKIGDIVGI |
| | | SGDTGFSTGPHLHYEMRRNGRHFNP |
| M23-S52_G26_FamH | 55 | GRHYGIDFQMPTGTNIYAVKGGIADKVWTDYGGGN |
| | | SIQIKTGANEWNWYMHLSKQLVRQGQRIKAGQLIG |
| | | KSGATGNFVRGAHLHFQLMQGSHPGNDTAKDP |
| M23-S53_G36_FamK | 56 | GVHHGRDITSGTIDGSPIKAARSGVVTFKGWTGGG |
| | | NTLSIFDGKNTYTYMHMKNPAKVVKGQRVKAGQIV |
| | | GNVGTTADRSLGGFSTGSHLHVQVNLGKTPSGTFM |
| | | N |
| M23-S54_G26_FamH | 57 | GRHYGIDFGMPTGTNVYAVKGGIADKVWTDYGGGN |
| | | SIQIKTGANEWNWYMHLSKQLARQGQRIKAGQLIG |
| | | KSGATGNFVRGAHLHFQLMQGSHPGNDTAKDP |
| M23-S55_G26_FamH | 58 | GRHYGIDFGMPTGTNIYAVKGGIADKVWTDYGGGN |
| | | SIQIKTGANEWNWYMHLSKQLARQGQRIKAGQLIG |
| | | KSGATGNFVRGAHLHFQLMQGSHPGNDTAKDP |
| M23-S56_G26_FamH | 59 | GRHYGVDFGMPAGTSIYAVKGGIADRVWTDFGGGN |
| | | SIQIKTGANEWNWYMHLSKQIARQGQRIKAGQLIG |
| | | LSGATGNFVKGAHLHFQLMRGSHPGNDTAVDP |
| M23-S57_G26_FamH | 60 | GRHYGIDFGMPSGTNVYAVKGGIADKVWTDYSGGN |
| | | SIQIKTGANEWNWYMHLSKQLARQGQRIKAGQLIG |
| | | KSGATGNFVRGAHLHFQLMQGSHPGNDTAKDP |
| M23-S58_G24_FamH | 61 | GRHYGMDFGMPAGTKIYAVKGGTVDNVWYDYGGGN |
| | | SIQIKTGPGEWNWYMHLSKQLVRLGEHIRTGQLIA |
| | | ESGATGAYCKGAHLHFQLMRGDHPGNDTAIDP |
| M23-S59_G2_FamA | 62 | RPHYGLDINYKHDKVYSTMSGTARTFNGWSGGFGR |
| | | HVEITNGNLKSIYGHLHKLAFNGTKKVKPGTFLGI |
| | | SGGDPREDGQNAGSSTGLHLHYEMQRNGRAFDP |
| M23-S60_G23_FamG | 63 | GKHYGIDFGMPTGTKIKALTDGKISQAGAVAGGGG |
| | | NQITLDEPGGKWYQWYMHMSKIIAKKGQKVSAGDV |
| | | IGLSGSTGNSTTPHLHIQRMKGYPSNETAVNP |
| M23-S61_G2_FamA | 64 | RPHYGLDINYKHDKVYSTMGGTATAARGWNGGFGN |
| | | FVTVTSGALKSIYGHLHKLAFTGTKKVKPGTFLGI |
| | | SGGDPREDGQGAGSSTGLHLHYEMQRNGRPFDP |
| M23-S62_G7_FamA | 65 | RPHHGIDVNYPYGTKVYSTIAGKATGSHGYNCGFG |
| | | NMMQIISGAIKVIYGHLSKLAFTGSKQVRPGSFLG |
| | | LSGGDPARQGAGAGSSTGPHLHYEMQWNGVPKDP |
| M23-S63_G22_FamF | 66 | GVHHGVDYDTPIGTPIRTPMGGRVRSWYDNYGGGK |
| | | AITVQKGRTFLWFMHLSEQLRRTGEQIKAGQLIGK |
| | | SGNTGSMTNYRHLHFQVNQGGESNRYSTDP |
| M23-S64_G13_FamD | 67 | PFHEGLDFDYIYEPVPSTINGRAQVMPFHNGGYGK |
| | | WVKIVKGALEVIYAHLSKYKVKTGQQVRVGQTVGI |
| | | SGNTGFSTGPHLHYEMRWNGMHRDP |
| M23-S65_G31_FamI | 68 | SGHAGIDYGAPTGTPIPSPIDGKVIQSWFSPNQPS |
| | | GGNETQIWDGQKYTHIFMHQSKRKVKTGDRVRQGQ |
| | | IIGLVGNTGNSFGSHLHWQVNKGKGYLNNHPDSVN |
| | | P |
| M23-S66_G24_FamH | 69 | GRHYGMDFGMPAGTSVYAVKGGTVDKLWYDYGGGN |
| | | SIQIKTGPGEWNWYMHLSKQLVKLGEQFKTGQLIA |
| | | KSGETGAYCKGAHLHFQLMRGDHPGNDTAIDP |
| M23-S67_G25_FamH | 70 | GRHYGIDFGMTPGTPVYAVKGGKARVFDDYGGGHS |
| | | VEIKTGANEWNWYMHLSKQIAKTGEQIKAGQLIAK |
| | | SGNTGAFTAGSGHLHFQLMQGSHPGNDTAKDP |
| M23-S68_G6_FamA | 71 | RAHHGLDIGYPYGTKVFSTTGGTATASKGWNGGFG |
| | | NMVSVKSGTMETIYGHLSKHAFSGSKKVKPGDLLG |
| | | LSGGDPSRQGREAGSSTGAHLHYEMRWGGVPKDP |
| M23-S69_G26_FamH | 72 | GKHYGMDFGMPTGTNIYAVKGGIADKVWTDFGGGN |
| | | SVQIKTAANEWNWYMHLSKQIARQGQRIKAGQLIG |
| | | KSGATGNFVRGAHLHFQLMRGGHPGNDTAVNP |
| M23-S70_G26_FamH | 73 | GKHYGMDFGMPTGTNVYAVKGGIADKVWTDFGGGN |
| | | SVQIKTAANEWNWYMHLSKQIARQGQRIKAGQLIG |
| | | KSGATGNFVRGAHLHFQLMRGGHPGNDTAVNP |
| M23-S71_G18_FamE | 74 | GHHYGIDFGAPFGTTINATNSGQLGELHNYGGGLV |
| | | ARLLTGQFTLFFMHLSKILKHGKVQAGEPIAKTGN |
| | | SGHWTTGAHLHFQVEKGRHNDITNQNTINP |
| M23-S72_G24_FamH | 75 | GRHYGMDFGMPAGTKVYAVKGGTVDNVWYDYGGGN |
| | | SIQIKTGPSEWNWYMHLSKQLVRLGEHIRTGQLIA |
| | | ESGATGAYCKGAHLHFQLMRGDHPGNDTAIDP |
| M23-S73_G31_FamI | 76 | GGHAGIDYGAATGTPIPSPIDGKVIQSWFSPNQPS |
| | | GGNETQIWDGHKYTHIFMHQSKRKVKTGDRVHQGQ |
| | | IIGLVGDTGNSFGSHLHWQVNKGKGYLNNHPDSI |
| M23-S74_G14_FamD | 77 | PFHEGVDFPFIYQTVRTPMGGRLTRMPFMAGGYGN |
| | | YVKITSGAIDMLFAHLKNFSKSPPSGSTVKPGDVV |
| | | GLTGNTGFSTGPHLHFEMRRNGRHFDP |
| M23-S75_G2_FamA | 78 | RPHYGLDINYKHDKVYSTLNGIANASYGWNGGFGN |
| | | MVKITSGALTAIYGHMHKLAFTGSKKVHPGSYLGI |
| | | SGGNPSEDGQGAGSSTGLHLHYEMQRNGVPFDP |
| M23-S76_G12_FamC | 79 | WPHPGIDLPYIYEPVYSTISGKAYTKEMPKGFGHY |
| | | IQVKGGALDVIYGHLSKWLVKNGQKVQPGTKLGIS |
| | | GNTGASTGPHLHYEMHKNGKPIDP |
| M23-S77_G2_FamA | 80 | RPHYGLDINYKHDKVYSTMSGTAKAFTGWSGGFGN |
| | | HMEVTNGNLKSIYGHLHKLAFHGTKKVKPGTFLGI |
| | | SGGDPREDGQGAGSSTGLHLHYEMQRNGRAFDP |
| M23-S78_G18_FamE | 81 | GHHYGIDFGTPYGTTINSTNDGNLKEIHNFGGGLV |
| | | ARLLTGQFTLFFMHLSKILKHGKVKAGEPIAKTGN |
| | | SGNWTTGPHLHFQVEKGRHDTITNANTVNP |
| M23-S79_G33_FamI | 82 | GGHAGIDYGAPTGTPIPSPIDGKVIKSWQSPWGGG |
| | | NETQVYDGNKYTHIFMHQSRRGVSAGDKVHQGEIL |
| | | GKVGSTGNSSGPHLHWQVNKGKGYLNNHPDS |
| M23-S80_G2_FamA | 83 | RPHHGLDINYKHDKVYSTMSGTAKATTGWGGGFGN |
| | | HVEITNGNLKSIYGHLHKLAFHGTKKVKPGTFLGI |
| | | SGGDPREDGQGAGSSTGLHLHYEMQRNGQPFDP |
| M23-S81_G36_FamK | 84 | GAHHGRDITSGTINGSPIKAARSGVVTFKGWTGGG |
| | | NTLSIFDGKNTYTYMHMKNPAKVVKGQRVKAGQIV |
| | | GNVGTTADRSLGGFSTGPHLHVQVNLGKTPSGTFM |
| | | NT |
| M23-S82_G18_FamE | 85 | GAHFGIDYGAPYGTTINATNDGVVKGIHNFGGGLV |
| | | ARLLTGQFTLFFMHLSKILKEGKIKAGEPMAKTGN |
| | | SGHWTTGPHLHFQVEKGRHDTITNANTVDP |
| M23-S83_G1_FamA | 86 | RAHHGLDINYKYDKVYSTLSGMATASSGWNGGFGQ |
| | | NVWIRAKNGLEAIYGHLHKLQFSGKKRVKPGDLLG |
| | | VSGGDPGRDGANAGSSTGPHLHYEMRKNGVPFAP |
| M23-S84_G16_FamE | 87 | GRHYGIDTPHQYEKIQAPTGGIVRKQYDPGGGTVA |
| | | QILNGKLAQYYLHLTDVLKTGRIKKGQTFAKTGNS |
| | | GANTTGPHLHTQIEDPAANALTNSNTKDP |
| M23-S85_G19_FamE | 88 | GKHFGIDYATPSGTTLKATNDGKVSKLHDHGGGTV |
| | | AKLLSGKFTQFFMHLSNVLKTGKVKQGEGFAKTGN |
| | | SGAWTTGPHLHYQVERGNSPFVTNKNTMDP |
| M23-S86_G31_FamI | 89 | NGHAGIDYGAATGTPIPSPIDGKVIQSWFSPNQPS |
| | | GGNETQIWDGHKYTHIFMHQSKRKVKTGDRVHQGQ |
| | | IIGLVGDTGNSFGSHLHWQVNKGKGYLNNHPDSI |
| M23-S87_G23_FamG | 90 | GRHYGVDFGMPTGTKIKALTDGKISQAGAVAGGGG |
| | | NQITLDEPGGKWYQWYMHMSKIIAKKGQKVSAGDV |
| | | IGLSGSTGNSTTPHLHIQRMKGYPSNETAVNP |
| M23-S88_G31_FamI | 91 | SGHAGIDYGAPTGTPIPSPIDGKVIQSWFSPNQPS |
| | | GGNETQIWDGQKYTHIFMHQSKRKVKIGDRVHQGQ |
| | | IIGLVGNTGNSFGSHLHWQVNKGKGYLNNHPDSVN |
| | | P |
| M23-S89_G5_FamA | 92 | GRHDGLDIGYPYGSKLYSTMSGTGTGSKGWNGGFG |
| | | NNMWLNKGGPVEAIYGHMSELAWTGKKKVKPGTYI |
| | | GKSGGDPARQGAGAGQSTGPHLHYEMRWNGQPKDP |
| M23-S90_G8_FamB | 93 | AHPGIDLPYRYEKVQTPLGGTIKTGEMPGGFGHYL |
| | | RVMAKPYDAYFGHLSKWLVKDGQHVSPGDAIAISG |
| | | NTGASTGPHLHFEMNKHGFGANTGHSIDP |
| M23-S91_G21_FamF | 94 | GVHYGIDYDMPENTPVRTPMGGKIRNWFDNGGGGN |
| | | AVTISKNGTYLWFMHLNKQLRKQGEIVKSGDLIAK |
| | | SGNTGSMTNYRHLHFQVMKGSESNRAAIDP |
| M23-S93_G26_FamH | 95 | GRHYGIDFQMPTGTNIYAVKGGIADKVWTDYGGGN |
| | | SIQIKTGANEWNWYMHLSKQLARQGQRIKAGQLIG |
| | | KSGATGNFVRGAHLHFQLMQGSHPGNDTAKDP |
| M23-S94_G2_FamA | 96 | RPHYGLDINYKHDKVYSTMSGTAKAFTGWSGGFGN |
| | | HMEVTNGNVKSIYGHLHKLAFHGTKKVKPGTFLGI |
| | | SGGDPREDGQGAGSSTGLHLHYEMQWNGQPKDP |
| M23-S95_G9_FamB | 97 | AHPGIDLPYIYEKVLTPMGGKVETRHTASGFGKHV |
| | | IVRAKPYDAYFGHLSKWLVKNGQRVKPGDAIGISG |
| | | DTGSSSGPHLHYEMNKHGFGSMTGHSIDP |
| M23-S96_G9_FamB | 98 | AHPGIDLPYIYEKVQTPLEGKVETRNTASGFGHHI |
| | | IVRAKPYDAYFGHLSKWLVKNGQHVKPGDTIGISG |
| | | NTGSSSGPHLHYEMNKHGFGSMTGHSIDP |
| M23-S97_G21_FamF | 99 | GVHYGVDYDIPENTPVRTPMGGRIRNWFDNGGGGN |
| | | TVTVSKNGTYLWFMHLNKQLRKNGETVKAGDLIAK |
| | | SGNTGSMTNYRHLHFQVMKGSESNSAAIDP |
| M23-S98_G24_FamH | 100 | GRHYGMDFGMPAGTKVYAVKGGTVDNVWYDYGGGN |
| | | SIQIKTGPSEWNWYMHLSKQLVRLGEYIRTGQLIA |
| | | ESGATGAYCKGAHLHFQLMRGDHPGNDTAIDP |
| M23-S99_G24_FamH | 101 | GRHYGMDFGMPAGTKVYAVKGGTVDNVWYDYGGGN |
| | | SIQIKTGPSEWNWYMHLSKQLVRLGEHIRTGQLIA |
| | | ESGATGAFCKGAHLHFQLMRGDHPGNDTAIDP |
| M23-S100_G29_FamH | 102 | GNHYGLDFGMPTGTSIKAVAGGKVSRVWNDYGGGK |
| | | SIEVALGGGLTNWYMHLSKQLVKQGQKVSVGDEIA |
| | | KSGATGNFVRGAHLHFQLNKNGKPQSN |
| M23-S101_G9_FamB | 103 | AHPGIDLPYIYEKVLTPMGGKVETRHTASGFGKHV |
| | | IVRAKPYDAYFGHLSKWLVKNGQHVKPGDAIGISG |
| | | NTGSSSGPHLHYEMNKHGFGSMTGHSIDP |
| M23-S102_G2_FamA | 104 | RPHFGLDINYKHDKVYSTMSGTARTFNGWSGGFGR |
| | | HVEITNGNLKSIYGHLHKLAFNGTKKVRPGTFLGI |
| | | SGGDPREDGQNAGSSTGLHLHYEMQRNGRAFDP |
| M23-S103_G8_FamB | 105 | AHPGIDLPYRNEKVQTPLGGTIKTGEMPGGFGHYL |
| | | RVMAKPYDAYFGHLSKWLVKDGQHVKPGDTIAISG |
| | | STGASTGPHLHFELNKHGFAANTGHSIDP |
| M23-S104_G23_FamG | 106 | GRHYGVDFGMPTGTKIKALTDGKISQAGAVAGGGG |
| | | NQITLDEPGGKWYQWYMHMSKIIAKKGQKVSAGDV |
| | | IGLSGNTGNSTTPHLHIQRMKGYPSNETAVNP |
| M23-S105_G3_FamA | 107 | RPHMGLDINYPYGSKLYSTLGGTATAKSGYNGGFG |
| | | NSMWIKSGVMQAIYGHMSELAFSGSKKVKPGDYLG |
| | | KSGGDPSKQGASAGDSTGAHLHYEMRRNGEPFDP |
| M23-S106_G2_FamA | 108 | RPHHGLDINYKHDKVYSTMSGTARAFKGWSGGFGN |
| | | HMEVTNGNVKSIYGHLHKLAFTGSKKVHPGTFLGI |
| | | SGGDPGEDGQGAGSSTGLHLHYEMQWGGVAKDP |
| M23-S107_G22_FamF | 109 | GVHHGVDYDTPTGTPIRTPMGGRVRSWYDNYGGGK |
| | | AITVQKGRTFLWFMHLSEQLRRTGEQIKAGQLIGK |
| | | SGNTGSMTNYRHLHFQVNEGGEANRYSTDP |
| M23-S108_G10_FamC | 110 | WPHPGIDLPYHHEPIFSTLNGKAYNKFMAGGFGKY |
| | | ILVKSGALEAFYAHLSKSFIKDGQSVHAGQKLGIS |
| | | GGDPGLAISGASTGPHLHYEMHRNGKPINP |
| M23-S109_G15_FamD | 111 | PFHEGVDFPFVYETIRTPMGGRVARQSFMAGGYGN |
| | | WVKVISGAMELIFAHLKNFSKTPPSGKNVKPGDVI |
| | | GLTGNTGFSTGPHLHFGIRKNGRDIDP |
| M23-S110_G26_FamH | 112 | GRHYGVDFGMPVGTNIYAVKGGIADHVWTDFGGGN |
| | | SIQIKTGANEWNWYMHLSKQIARQGQRIKAGQLIG |
| | | LSGATGNFVRGAHLHFQLMRGSHPGNDTAVDP |
| M23-S111_G15_FamD | 113 | PFHEGVDFPFVYQTVRTPMGGRVARQSFMPGGYGN |
| | | WVKVISGAMELIFAHLKNFSKTPASGKNVKPGDVI |
| | | GLTGNTGFSTGPHLHFGIRKNGRDIDP |
| M23-S112_G34_FamJ | 114 | TPHYGVDYASSVGSPIRAARGGVVLSSGWSNYGGG |
| | | NQIVIYNPKLNKTFTYMHMLSDLRVKKGQQVVAGQ |
| | | LIGRMGNTGNSSGPHLHFQVNEGKGFTAAGT |
| M23-S113_G31_FamI | 115 | NGHAGIDYGAATGTPIPSPIDGKVIQSWFSPNQPS |
| | | GGNETQIWDGHKYTHIFMHQSKRKVKTGDRVHQGQ |
| | | IIGLVGDTGNSFGSHLHWQVNKGKGYLNNHPDSIN |
| | | P |
| M23-S114_G20_FamE | 116 | GRHYGIDYATPVGTTLTAPTSGTISKLSNYGGGTV |
| | | AKLLSGKFTQFFMHLKDIFKTGKVKQGEKFAHTGN |
| | | SGHWTTGPHLHYQVEKGHSADITNRNTVDP |
| M23-S115_G26_FamH | 117 | GRHYGIDFGMPTGTNVYAVKGGIADKVWTDYGGGN |
| | | SIQIKTGANEWNWYMHLSKQLARQGQRIKAGQLIG |
| | | KSGATGNFVRGAHLHLQLMQGSHPGNDTAKDP |
| M23-S116_G26_FamH | 118 | GRHYGIDFGMPTGTNIYAVKGGIADKVWTDYGGGN |
| | | SIQIKTGANEWNWYMHLSKQLVRQGQRIKAGQLIG |
| | | KSGATGNFVRGAHLHFQLMQGSHPGNDTAKDP |
| M23-S117_G9_FamB | 119 | AHPGIDLPYIYEKVQTPLEGKVQTKDTGNVGFGHH |
| | | VVVKAKPYDAYFGHMSKWSVKNGQHVKPGDVLGIS |
| | | GNTGSSTGPHLHYEMNKHGMGSMTGHSIDP |
| M23-S118_G18_FamE | 120 | GHHYGIDFGAPFGTTINATNSGQLGELHNFGGGLV |
| | | ARLLTGQFTLFFMHLSKILKHGKVQAGEPIAKTGN |
| | | SGNWTTGPHLHFQVEKGRHNDITNQNTVNP |
| M23-S119_G20_FamE | 121 | GRHFGIDYGTPAGTVIKAPTSGTVSTLHNYGGGLV |
| | | AKLLSGKFTQFFMHLQDILKTGRVKKGDKFAKTGN |
| | | SGHWTTGAHLHYQVEKGNSSDITNKNTVDP |
| M23-S120_G13_FamD | 122 | PFHEGLDFDYIYEPVPSTINGRAQVMPFHNGGYGK |
| | | WVKIVKGALEVIYAHLSKYKVKTGHQVRVGQTVGI |
| | | SGNTGFSTGPHLHYEMRWNGRHRDP |
| M23-S121_G26_FamH | 123 | GRHYGIDFGMPTGTNIYAVKGGIADKVWTDYGGGN |
| | | SIQIKTGANEWNWYMHLSKQLARQGQRIKAGQLIG |
| | | KSGATGNFVRGAHLHFQLMRGSHPGNDTAVDP |

In some embodiments, the EAD has the sequence of any one of SEQ ID NO: 4-123. In some embodiments, the EAD has at least 80, 81, 82, 83, 84, 85, 86, 87, 88, 89, 90, 91, 92, 93, 94, 95, 96, 97, 98, or 99% identity with the sequence of any one of SEQ ID NO: 4-123. In some embodiments, the EAD differs from the sequence of any one of SEQ ID NO: 4-123 by 1, 2, 3, 4, 5, 6, 7, 8, 9, 10, 11, 12, 13, 14, or 15 amino acids.

In some embodiments, the EAD is an M23 sequence of the family A, B, C, D, E, F, G, H, I, J, or K herein. In some embodiments, the EAD has at least 80, 81, 82, 83, 84, 85, 86, 87, 88, 89, 90, 91, 92, 93, 94, 95, 96, 97, 98, or 99% identity with an M23 sequence of the family A, B, C, D, E, F, G, H, I, J, or K herein. In some embodiments, the EAD differs from an M23 sequence of the family A, B, C, D, E, F, G, H, I, J, or K herein by 1, 2, 3, 4, 5, 6, 7, 8, 9, 10, 11, 12, 13, 14, or 15 amino acids.

In some embodiments, the EAD is an M23 sequence of the group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 herein. In some embodiments, the EAD has at least 80, 81, 82, 83, 84, 85, 86, 87, 88, 89, 90, 91, 92, 93, 94, 95, 96, 97, 98, or 99% identity with an M23 sequence of the group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 herein. In some embodiments, the EAD differs from an M23 sequence of the group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 herein by 1, 2, 3, 4, 5, 6, 7, 8, 9, 10, 11, 12, 13, 14, or 15 amino acids.

In some embodiments, the EAD is an M23 sequence of the E, F, H, I, or J families described herein. In some embodiments, the EAD has at least 80, 81, 82, 83, 84, 85, 86, 87, 88, 89, 90, 91, 92, 93, 94, 95, 96, 97, 98, or 99% identity with an M23 sequence of the E, F, H, I, or J families. In some embodiments, the EAD differs from an M23 sequence of the E, F, H, I, or J families by 1, 2, 3, 4, 5, 6, 7, 8, 9, 10, 11, 12, 13, 14, or 15 amino acids.

In some embodiments, the EAD is an M23 sequence of the group 17, 21, 24, 25, 26, 29, 30, 31, 32, 33, or 34 described herein. In some embodiments, the EAD has at least 80, 81, 82, 83, 84, 85, 86, 87, 88, 89, 90, 91, 92, 93, 94, 95, 96, 97, 98, or 99% identity with an M23 sequence of the group 17, 21, 24, 25, 26, 29, 30, 31, 32, 33, or 34. In some embodiments, the EAD differs from an M23 sequence of the group 17, 21, 24, 25, 29, 30, 31, 32, 33, or 34 by 1, 2, 3, 4, 5, 6, 7, 8, 9, 10, 11, 12, 13, 14, or 15 amino acids.

Strikingly, all EADs tested from groups 17, 21, 24, 25, 29, 30, 31, 32, 33, and 34 displayed S. aureus antimicrobial activity in chimeric proteins in combination with the LysH5 CBD, as disclosed in Example 12. In some embodiments, the EAD is an M23 sequence of the group 17, 21, 24, 25, 29, 30, 31, 32, 33, or 34 described herein. In some embodiments, the EAD has at least 80, 81, 82, 83, 84, 85, 86, 87, 88, 89, 90, 91, 92, 93, 94, 95, 96, 97, 98, or 99% identity with an M23 sequence of the group 17, 21, 24, 25, 29, 30, 31, 32, 33, or 34. In some embodiments, the EAD differs from an M23 sequence of the group 17, 21, 24, 25, 29, 30, 31, 32, 33, or 34 by 1, 2, 3, 4, 5, 6, 7, 8, 9, 10, 11, 12, 13, 14, or 15 amino acids.

In some embodiments, the EAD has the sequence of any one of M23-SI (SEQ ID NO: 4), M23-S3 (SEQ ID NO: 6), M23-S6 (SEQ ID NO: 9), M23-S20 (SEQ ID NO: 23), M23-S21 (SEQ ID NO: 24), M23-S28 (SEQ ID NO: 31), M23-S47 (SEQ ID NO: 50), M23-S56 (SEQ ID NO: 59), M23-S65 (SEQ ID NO: 68), M23-S66 (SEQ ID NO: 69), M23-S67 (SEQ ID NO: 70), M23-S69 (SEQ ID NO: 72), M23-S73 (SEQ ID NO: 76), M23-S79 (SEQ ID NO: 82), M23-S91 (SEQ ID NO: 94), M23-S97 (SEQ ID NO: 99), M23-S99 (SEQ ID NO: 101), M23-S100 (SEQ ID NO: 102), or M23-S112 (SEQ ID NO: 114). In some embodiments, the EAD has at least 80, 81, 82, 83, 84, 85, 86, 87, 88, 89, 90, 91, 92, 93, 94, 95, 96, 97, 98, or 99% identity with the sequence of any one of M23-SI (SEQ ID NO: 4), M23-S3 (SEQ ID NO: 6), M23-S6 (SEQ ID NO: 9), M23-S20 (SEQ ID NO: 23), M23-S21 (SEQ ID NO: 24), M23-S28 (SEQ ID NO: 31), M23-S47 (SEQ ID NO: 50), M23-S56 (SEQ ID NO: 59), M23-S65 (SEQ ID NO: 68), M23-S66 (SEQ ID NO: 69), M23-S67 (SEQ ID NO: 70), M23-S69 (SEQ ID NO: 72), M23-S73 (SEQ ID NO: 76), M23-S79 (SEQ ID NO: 82), M23-S91 (SEQ ID NO: 94), M23-S97 (SEQ ID NO: 99), M23-S99 (SEQ ID NO: 101), M23-S100 (SEQ ID NO: 102), or M23-S112 (SEQ ID NO: 114). In some embodiments, the EAD differs from the sequence of any one of M23-SI (SEQ ID NO: 4), M23-S3 (SEQ ID NO: 6), M23-S6 (SEQ ID NO: 9), M23-S20 (SEQ ID NO: 23), M23-S21 (SEQ ID NO: 24), M23-S28 (SEQ ID NO: 31), M23-S47 (SEQ ID NO: 50), M23-S56 (SEQ ID NO: 59), M23-S65 (SEQ ID NO: 68), M23-S66 (SEQ ID NO: 69), M23-S67 (SEQ ID NO: 70), M23-S69 (SEQ ID NO: 72), M23-S73 (SEQ ID NO: 76), M23-S79 (SEQ ID NO: 82), M23-S91 (SEQ ID NO: 94), M23-S97 (SEQ ID NO: 99), M23-S99 (SEQ ID NO: 101), M23-S100 (SEQ ID NO: 102), or M23-S112 (SEQ ID NO: 114) by 1, 2, 3, 4, 5, 6, 7, 8, 9, 10, 11, 12, 13, 14, or 15 amino acids.

In some embodiments, the EAD is an M23 sequence of the H family herein, the same family as the M23-S1 and M23-S66 sequences. In some embodiments, the EAD has at least 80, 81, 82, 83, 84, 85, 86, 87, 88, 89, 90, 91, 92, 93, 94, 95, 96, 97, 98, or 99% identity with an M23 sequence of the H family herein. In some embodiments, the EAD differs from an M23 sequence of the H family herein by 1, 2, 3, 4, 5, 6, 7, 8, 9, 10, 11, 12, 13, 14, or 15 amino acids.

In some embodiments, the EAD is an M23 sequence of group 26 herein, the same group as the M23-SI sequence. In some embodiments, the EAD has at least 80, 81, 82, 83, 84, 85, 86, 87, 88, 89, 90, 91, 92, 93, 94, 95, 96, 97, 98, or 99% identity with an M23 sequence of group 26 herein. In some embodiments, the EAD differs from an M23 sequence of group 26 herein by 1, 2, 3, 4, 5, 6, 7, 8, 9, 10, 11, 12, 13, 14, or 15 amino acids.

In some embodiments, the EAD is an M23 sequence of group 24 herein, the same group as the M23-S66 sequence. In some embodiments, the EAD has at least 80, 81, 82, 83, 84, 85, 86, 87, 88, 89, 90, 91, 92, 93, 94, 95, 96, 97, 98, or 99% identity with an M23 sequence of group 24 herein. In some embodiments, the EAD differs from an M23 sequence of group 24 herein by 1, 2, 3, 4, 5, 6, 7, 8, 9, 10, 11, 12, 13, 14, or 15 amino acids.

Sequence identity among the sequences in Families A-K is shown in Tables 2-12. Between sequences from two different families, sequence identity is less than 50%.

TABLE 2
Sequence identity among sequences in M23 EAD Family A.

| Group | | S8 | S83 | S75 | S61 | S106 | S59 | S31 | S102 | S80 | S77 |
|---|---|---|---|---|---|---|---|---|---|---|---|
| 1 | S8 | 100.0 | 88.5 | 73.1 | 71.2 | 68.3 | 71.2 | 70.2 | 70.2 | 73.1 | 71.2 |
| 1 | S83 | 88.5 | 100.0 | 67.3 | 66.4 | 62.5 | 65.4 | 64.4 | 64.4 | 67.3 | 63.5 |
| 2 | S75 | 73.1 | 67.3 | 100.0 | 81.6 | 74.8 | 74.8 | 73.8 | 73.8 | 77.7 | 75.7 |
| 2 | S61 | 71.2 | 66.4 | 81.6 | 100.0 | 80.6 | 85.4 | 83.5 | 83.5 | 87.4 | 88.4 |
| 2 | S106 | 68.3 | 62.5 | 74.8 | 80.6 | 100.0 | 84.5 | 83.5 | 84.5 | 84.5 | 88.4 |
| 2 | S59 | 71.2 | 65.4 | 74.8 | 85.4 | 84.5 | 100.0 | 97.1 | 98.1 | 89.3 | 92.2 |
| 2 | S31 | 70.2 | 64.4 | 73.8 | 83.5 | 83.5 | 97.1 | 100.0 | 99.0 | 89.3 | 91.3 |
| 2 | S102 | 70.2 | 64.4 | 73.8 | 83.5 | 84.5 | 98.1 | 99.0 | 100.0 | 88.4 | 90.3 |
| 2 | S80 | 73.1 | 67.3 | 77.7 | 87.4 | 84.5 | 89.3 | 89.3 | 88.4 | 100.0 | 93.2 |
| 2 | S77 | 71.2 | 63.5 | 75.7 | 88.4 | 88.4 | 92.2 | 91.3 | 90.3 | 93.2 | 100.0 |
| 2 | S94 | 69.2 | 62.5 | 73.8 | 85.4 | 90.3 | 87.4 | 86.4 | 85.4 | 92.2 | 95.2 |
| 3 | S105 | 66.7 | 64.8 | 67.3 | 65.4 | 58.7 | 59.6 | 58.7 | 58.7 | 62.5 | 61.5 |
| 4 | S29 | 69.2 | 50.0 | 55.3 | 54.4 | 53.4 | 52.4 | 53.4 | 53.4 | 51.5 | 53.4 |
| 4 | S41 | 57.7 | 55.8 | 60.2 | 55.3 | 53.4 | 51.5 | 51.5 | 51.5 | 53.4 | 52.4 |
| 4 | S24 | 58.7 | 54.8 | 58.3 | 51.5 | 52.4 | 48.5 | 48.5 | 48.5 | 51.5 | 50.5 |
| 4 | S36 | 58.7 | 52.9 | 59.2 | 55.3 | 52.4 | 49.5 | 49.5 | 49.5 | 52.4 | 51.5 |
| 5 | S89 | 66.7 | 61.0 | 57.1 | 59.1 | 60.0 | 54.3 | 53.3 | 53.3 | 58.1 | 57.1 |
| 6 | S15 | 63.8 | 65.7 | 63.5 | 65.4 | 65.4 | 57.7 | 56.7 | 56.7 | 61.5 | 59.6 |
| 6 | S68 | 64.8 | 66.7 | 63.5 | 65.4 | 65.4 | 57.7 | 56.7 | 56.7 | 61.5 | 59.6 |
| 7 | S43 | 68.6 | 65.7 | 63.5 | 65.4 | 68.3 | 59.6 | 58.7 | 58.7 | 61.5 | 63.5 |
| 7 | S62 | 64.8 | 61.0 | 66.4 | 65.4 | 65.4 | 58.7 | 59.6 | 59.6 | 62.5 | 60.6 |
| Group | S94 | S105 | S29 | S41 | S24 | S36 | S89 | S15 | S68 | S43 | S62 |
| 1 | 69.2 | 66.7 | 69.2 | 57.7 | 58.7 | 58.7 | 66.7 | 63.8 | 64.8 | 68.6 | 64.8 |
| 1 | 62.5 | 64.8 | 50.0 | 55.8 | 54.8 | 52.9 | 61.0 | 65.7 | 66.7 | 65.7 | 61.0 |
| 2 | 73.8 | 67.3 | 55.3 | 60.2 | 58.3 | 59.2 | 57.1 | 63.5 | 63.5 | 63.5 | 66.4 |
| 2 | 85.4 | 65.4 | 54.4 | 55.3 | 51.5 | 55.3 | 59.1 | 65.4 | 65.4 | 65.4 | 65.4 |
| 2 | 90.3 | 58.7 | 53.4 | 53.4 | 52.4 | 52.4 | 60.0 | 65.4 | 65.4 | 68.3 | 65.4 |
| 2 | 87.4 | 59.6 | 52.4 | 51.5 | 48.5 | 49.5 | 54.3 | 57.7 | 57.7 | 59.6 | 58.7 |
| 2 | 86.4 | 58.7 | 53.4 | 51.5 | 48.5 | 49.5 | 53.3 | 56.7 | 56.7 | 58.7 | 59.6 |
| 2 | 85.4 | 58.7 | 53.4 | 51.5 | 48.5 | 49.5 | 53.3 | 56.7 | 56.7 | 58.7 | 59.6 |
| 2 | 92.2 | 62.5 | 51.5 | 53.4 | 51.5 | 52.4 | 58.1 | 61.5 | 61.5 | 61.5 | 62.5 |
| 2 | 95.2 | 61.5 | 53.4 | 52.4 | 50.5 | 51.5 | 57.1 | 59.6 | 59.6 | 63.5 | 60.6 |
| 2 | 100.0 | 60.6 | 51.5 | 53.4 | 51.5 | 52.4 | 61.9 | 62.5 | 62.5 | 64.4 | 63.5 |
| 3 | 60.6 | 100.0 | 56.7 | 68.3 | 60.6 | 64.4 | 70.5 | 72.1 | 71.2 | 66.4 | 65.4 |
| 4 | 51.5 | 56.7 | 100.0 | 82.1 | 83.2 | 84.2 | 54.3 | 50.0 | 50.0 | 60.6 | 59.6 |
| 4 | 53.4 | 68.3 | 82.1 | 100.0 | 88.4 | 90.5 | 56.2 | 54.8 | 54.8 | 55.8 | 57.7 |
| 4 | 51.5 | 60.6 | 83.2 | 88.4 | 100.0 | 91.6 | 56.2 | 51.9 | 51.9 | 60.6 | 58.7 |
| 4 | 52.4 | 64.4 | 84.2 | 90.5 | 91.6 | 100.0 | 58.1 | 52.9 | 52.9 | 58.7 | 58.7 |
| 5 | 61.9 | 70.5 | 54.3 | 56.2 | 56.2 | 58.1 | 100.0 | 64.8 | 65.7 | 71.4 | 63.8 |
| 6 | 62.5 | 72.1 | 50.0 | 54.8 | 51.9 | 52.9 | 64.8 | 100.0 | 99.0 | 75.0 | 68.3 |
| 6 | 62.5 | 71.2 | 50.0 | 54.8 | 51.9 | 52.9 | 65.7 | 99.0 | 100.0 | 76.0 | 69.2 |
| 7 | 64.4 | 66.4 | 60.6 | 55.8 | 60.6 | 58.7 | 71.4 | 75.0 | 76.0 | 100.0 | 83.7 |
| 7 | 63.5 | 65.4 | 59.6 | 57.7 | 58.7 | 58.7 | 63.8 | 68.3 | 69.2 | 83.7 | 100.0 |

TABLE 3
Sequence identity among sequences in M23 EAD Family D.

| Group | | S51 | S64 | S13 | S14 | S120 | S32 | S19 | S2 | S12 | S74 | S26 | S33 | S45 | S49 | S109 | S111 |
|---|---|---|---|---|---|---|---|---|---|---|---|---|---|---|---|---|---|
| 13 | S51 | 100.0 | 81.1 | 81.1 | 81.1 | 81.1 | 82.1 | 53.6 | 54.6 | 55.7 | 56.7 | 55.7 | 55.7 | <50 | <50 | <50 | <50 |
| 13 | S64 | 81.1 | 100.0 | 97.9 | 97.9 | 97.9 | 99.0 | 51.6 | 51.6 | 50.5 | 53.6 | 52.6 | 52.6 | <50 | <50 | <50 | <50 |
| 13 | S13 | 81.1 | 97.9 | 100.0 | 97.9 | 97.9 | 99.0 | 51.6 | 52.6 | 51.6 | 53.6 | 52.6 | 52.6 | <50 | <50 | <50 | <50 |
| 13 | S14 | 81.1 | 97.9 | 97.9 | 100.0 | 97.9 | 99.0 | 50.5 | 51.6 | 50.5 | 53.6 | 52.6 | 52.6 | <50 | <50 | <50 | <50 |
| 13 | S120 | 81.1 | 97.9 | 97.9 | 97.9 | 100.0 | 99.0 | 51.6 | 52.6 | 51.6 | 54.6 | 53.6 | 53.6 | <50 | <50 | <50 | <50 |
| 13 | S32 | 82.1 | 99.0 | 99.0 | 99.0 | 99.0 | 100.0 | 51.6 | 52.6 | 51.6 | 54.6 | 53.6 | 63.6 | <50 | <50 | <50 | <50 |
| 14 | S19 | 53.6 | 51.6 | 51.6 | 50.5 | 51.6 | 51.6 | 100.0 | 99.0 | 97.9 | 92.8 | 93.8 | 93.8 | 69.1 | 70.1 | 73.2 | 74.2 |
| 14 | S2 | 54.6 | 51.6 | 52.6 | 51.6 | 52.6 | 52.6 | 99.0 | 100.0 | 99.0 | 93.8 | 94.9 | 94.9 | 70.1 | 71.1 | 74.2 | 75.3 |
| 14 | S12 | 55.7 | 50.5 | 51.6 | 50.5 | 51.6 | 51.6 | 97.9 | 99.0 | 100.0 | 92.8 | 93.2 | 93.8 | 69.1 | 70.1 | 73.2 | 74.2 |
| 14 | S74 | 56.7 | 53.6 | 53.6 | 53.6 | 54.6 | 54.6 | 92.8 | 93.8 | 92.8 | 100.0 | 96.9 | 97.9 | 72.2 | 73.2 | 76.3 | 76.3 |
| 14 | S26 | 55.7 | 52.6 | 52.6 | 52.6 | 53.6 | 53.6 | 93.8 | 94.9 | 93.2 | 96.9 | 100.0 | 97.9 | 74.2 | 75.3 | 75.3 | 76.3 |
| 14 | S33 | 55.7 | 52.6 | 52.6 | 52.6 | 53.6 | 63.6 | 93.8 | 94.9 | 93.8 | 97.9 | 97.9 | 100.0 | 73.2 | 74.2 | 76.3 | 77.3 |
| 15 | S45 | <50 | <50 | <50 | <50 | <50 | <50 | 69.1 | 70.1 | 69.1 | 72.2 | 74.2 | 73.2 | 100.0 | 99.0 | 86.7 | 87.6 |
| 15 | S49 | <50 | <50 | <50 | <50 | <50 | <50 | 70.1 | 71.1 | 70.1 | 73.2 | 75.3 | 74.2 | 99.0 | 100.0 | 90.7 | 88.7 |
| 15 | S109 | <50 | <50 | <50 | <50 | <50 | <50 | 73.2 | 74.2 | 73.2 | 76.3 | 75.3 | 76.3 | 86.7 | 90.7 | 100.0 | 95.9 |
| 15 | S111 | <50 | <50 | <50 | <50 | <50 | <50 | 74.2 | 75.3 | 74.2 | 76.3 | 76.3 | 77.3 | 87.6 | 88.7 | 95.9 | 100.0 |

TABLE 4
Sequence identity among sequences in M23 EAD Family H.

| Grp | | S66 | S58 | S98 | S72 | S5 | S99 | S67 | S50 | S17 | S69 | S70 | S56 | S110 | S46 |
|---|---|---|---|---|---|---|---|---|---|---|---|---|---|---|---|
| 24 | S66 | 100 | 90 | 90 | 90 | 90 | 39 | 71 | 74 | 72 | 71 | 72 | 75 | 72 | 73 |
| 24 | S58 | 90 | 100 | 97 | 98 | 98 | 97 | 68 | 71 | 74 | 73 | 72 | 77 | 75 | 76 |
| 24 | S98 | 90 | 97 | 100 | 99 | 97 | 98 | 69 | 71 | 73 | 72 | 73 | 76 | 75 | 75 |
| 24 | S72 | 90 | 98 | 99 | 100 | 98 | 99 | 69 | 71 | 73 | 72 | 73 | 76 | 75 | 75 |
| 24 | S5 | 90 | 98 | 97 | 98 | 100 | 99 | 70 | 73 | 74 | 73 | 74 | 77 | 75 | 76 |
| 24 | S99 | 89 | 97 | 98 | 99 | 99 | 100 | 70 | 72 | 73 | 73 | 74 | 77 | 75 | 76 |
| 25 | S67 | 71 | 68 | 69 | 69 | 70 | 70 | 100 | 76 | 74 | 71 | 72 | 74 | 73 | 74 |
| 26 | S50 | 74 | 71 | 71 | 71 | 73 | 72 | 76 | 100 | 84 | 82 | 83 | 82 | 84 | 84 |
| 26 | S17 | 72 | 74 | 73 | 73 | 74 | 73 | 74 | 84 | 100 | 91 | 90 | 85 | 86 | 86 |
| 26 | S69 | 71 | 73 | 72 | 72 | 73 | 73 | 71 | 82 | 91 | 100 | 99 | 89 | 91 | 91 |
| 26 | S70 | 72 | 72 | 73 | 73 | 74 | 74 | 72 | 83 | 90 | 99 | 100 | 88 | 90 | 90 |
| 26 | S56 | 75 | 77 | 76 | 76 | 77 | 77 | 74 | 82 | 85 | 89 | 88 | 100 | 96 | 98 |
| 26 | S110 | 72 | 75 | 75 | 75 | 75 | 75 | 73 | 84 | 86 | 91 | 90 | 96 | 100 | 98 |
| 26 | S46 | 73 | 76 | 75 | 75 | 76 | 76 | 74 | 84 | 86 | 91 | 90 | 98 | 98 | 100 |
| 26 | S47 | 75 | 76 | 75 | 75 | 76 | 76 | 76 | 85 | 84 | 87 | 86 | 89 | 91 | 91 |
| 26 | S3 | 75 | 76 | 75 | 75 | 76 | 76 | 74 | 84 | 88 | 91 | 90 | 91 | 93 | 93 |
| 26 | S121 | 75 | 77 | 76 | 76 | 77 | 77 | 75 | 85 | 89 | 92 | 91 | 92 | 94 | 94 |
| 26 | S57 | 75 | 75 | 75 | 75 | 76 | 76 | 77 | 86 | 84 | 87 | 88 | 88 | 90 | 90 |
| 26 | S1 | 75 | 75 | 76 | 76 | 77 | 77 | 78 | 87 | 85 | 88 | 89 | 89 | 91 | 91 |
| 26 | S115 | 75 | 75 | 75 | 75 | 76 | 76 | 77 | 86 | 85 | 88 | 89 | 88 | 90 | 90 |
| 26 | S54 | 75 | 75 | 76 | 76 | 77 | 77 | 78 | 87 | 86 | 89 | 90 | 89 | 91 | 91 |
| 26 | S93 | 74 | 75 | 75 | 75 | 75 | 75 | 76 | 85 | 86 | 89 | 88 | 89 | 91 | 91 |
| 26 | S55 | 75 | 76 | 75 | 75 | 76 | 76 | 77 | 86 | 87 | 90 | 89 | 90 | 92 | 92 |
| 26 | S116 | 75 | 77 | 76 | 76 | 77 | 77 | 76 | 85 | 86 | 89 | 88 | 89 | 91 | 91 |
| 26 | S52 | 75 | 76 | 75 | 75 | 76 | 76 | 75 | 84 | 85 | 88 | 87 | 88 | 90 | 90 |
| 27 | S42 | 62 | 59 | 60 | 60 | 61 | 61 | 62 | 71 | 69 | 65 | 65 | 64 | 64 | 64 |
| 28 | S10 | 56 | 54 | 55 | 55 | 56 | 56 | 64 | 60 | 56 | 56 | 57 | 56 | 56 | 57 |
| 29 | S100 | 53 | 53 | 52 | 52 | 53 | 53 | 50 | 57 | 58 | 54 | 53 | 55 | 54 | 55 |
| | Grp | S47 | S3 | S121 | S57 | S1 | S115 | S54 | S93 | S55 | S116 | S52 | S42 | S10 | S100 |
| | 24 | 75 | 75 | 75 | 75 | 75 | 75 | 75 | 74 | 75 | 75 | 75 | 62 | 56 | 53 |
| | 24 | 76 | 76 | 77 | 75 | 75 | 75 | 75 | 75 | 76 | 77 | 76 | 59 | 54 | 53 |
| | 24 | 75 | 75 | 76 | 75 | 76 | 75 | 76 | 75 | 75 | 76 | 75 | 60 | 55 | 52 |
| | 24 | 75 | 75 | 76 | 75 | 76 | 75 | 76 | 75 | 75 | 76 | 75 | 60 | 55 | 52 |
| | 24 | 76 | 76 | 77 | 76 | 77 | 76 | 77 | 75 | 76 | 77 | 76 | 61 | 56 | 53 |
| | 24 | 76 | 76 | 77 | 76 | 77 | 76 | 77 | 75 | 76 | 77 | 76 | 61 | 56 | 53 |
| | 25 | 76 | 74 | 75 | 77 | 78 | 77 | 78 | 76 | 77 | 76 | 75 | 62 | 64 | 50 |
| | 26 | 85 | 84 | 85 | 86 | 87 | 86 | 87 | 85 | 86 | 85 | 84 | 71 | 60 | 57 |
| | 26 | 84 | 88 | 89 | 84 | 85 | 85 | 86 | 86 | 87 | 86 | 85 | 69 | 56 | 58 |
| | 26 | 87 | 91 | 92 | 87 | 88 | 88 | 89 | 89 | 90 | 89 | 88 | 65 | 56 | 54 |
| | 26 | 86 | 90 | 91 | 88 | 89 | 89 | 90 | 88 | 89 | 88 | 87 | 65 | 57 | 53 |
| | 26 | 89 | 91 | 92 | 88 | 89 | 88 | 89 | 89 | 90 | 89 | 88 | 64 | 56 | 55 |
| | 26 | 91 | 93 | 94 | 90 | 91 | 90 | 91 | 91 | 92 | 91 | 90 | 64 | 56 | 54 |
| | 26 | 91 | 93 | 94 | 90 | 91 | 90 | 91 | 91 | 92 | 91 | 90 | 64 | 57 | 55 |
| | 26 | 100 | 93 | 94 | 94 | 95 | 94 | 95 | 95 | 96 | 97 | 96 | 65 | 62 | 57 |
| | 26 | 93 | 100 | 99 | 94 | 95 | 95 | 96 | 98 | 97 | 96 | 87 | 67 | 58 | 57 |
| | 26 | 94 | 99 | 100 | 95 | 96 | 96 | 97 | 97 | 98 | 97 | 96 | 68 | 59 | 58 |
| | 26 | 94 | 94 | 95 | 100 | 99 | 97 | 98 | 96 | 97 | 96 | 95 | 68 | 60 | 55 |
| | 26 | 95 | 95 | 96 | 99 | 100 | 98 | 99 | 97 | 98 | 97 | 96 | 69 | 61 | 56 |
| | 26 | 94 | 95 | 96 | 97 | 98 | 100 | 99 | 97 | 98 | 97 | 96 | 68 | 60 | 56 |
| | 26 | 95 | 96 | 97 | 98 | 99 | 99 | 100 | 98 | 99 | 98 | 97 | 69 | 61 | 57 |
| | 26 | 95 | 98 | 97 | 96 | 97 | 97 | 98 | 100 | 99 | 98 | 99 | 67 | 59 | 57 |
| | 26 | 96 | 97 | 98 | 97 | 98 | 98 | 99 | 99 | 100 | 99 | 98 | 68 | 60 | 58 |
| | 26 | 97 | 96 | 97 | 96 | 97 | 97 | 98 | 98 | 99 | 100 | 99 | 67 | 61 | 59 |
| | 26 | 96 | 87 | 96 | 95 | 96 | 96 | 97 | 99 | 98 | 99 | 100 | 66 | 60 | 58 |
| | 27 | 65 | 67 | 68 | 68 | 69 | 68 | 69 | 67 | 68 | 67 | 66 | 100 | 57 | 66 |
| | 28 | 62 | 58 | 59 | 60 | 61 | 60 | 61 | 59 | 60 | 61 | 60 | 57 | 100 | 72 |
| | 29 | 57 | 57 | 58 | 55 | 56 | 56 | 57 | 57 | 58 | 59 | 58 | 66 | 72 | 100 |

TABLE 5
Sequence identity among sequences in M23 EAD Family B.

| Group | | S103 | S18 | S90 | S30 | S117 | S96 | S95 | S101 |
|---|---|---|---|---|---|---|---|---|---|
| 8 | S103 | 100.0 | 91.9 | 93.9 | 63.0 | 69.0 | 73.0 | 69.0 | 70.0 |
| 8 | S18 | 91.9 | 100.0 | 98.0 | 65.0 | 71.0 | 74.0 | 71.0 | 73.0 |
| 8 | S90 | 93.9 | 98.0 | 100.0 | 65.0 | 72.0 | 75.0 | 72.0 | 74.0 |
| 9 | S30 | 63.0 | 65.0 | 65.0 | 100.0 | 79.0 | 74.0 | 70.0 | 71.0 |
| 9 | S117 | 69.0 | 71.0 | 72.0 | 79.0 | 100.0 | 85.0 | 80.0 | 82.0 |
| 9 | S96 | 73.0 | 74.0 | 75.0 | 74.0 | 85.0 | 100.0 | 90.9 | 92.9 |
| 9 | S95 | 69.0 | 71.0 | 72.0 | 70.0 | 80.0 | 90.9 | 100.0 | 98.0 |
| 9 | S101 | 70.0 | 73.0 | 74.0 | 71.0 | 82.0 | 92.9 | 98.0 | 100.0 |

TABLE 6
Sequence identity among sequences in M23 EAD Family C.

| Group | | S108 | S40 | S76 |
|---|---|---|---|---|
| 10 | S108 | 100.0 | 67.3 | 65.0 |
| 11 | S40 | 67.3 | 100.0 | 65.4 |
| 12 | S76 | 65.0 | 65.4 | 100.0 |

TABLE 7
Sequence identity among sequences in M23 EAD Family E.

| Grp | | S84 | S6 | S78 | S71 | S7 | S118 | S38 | S82 | S85 | S114 | S119 |
|---|---|---|---|---|---|---|---|---|---|---|---|---|
| 16 | S84 | 100.0 | 54.0 | <50 | <50 | <50 | <50 | <50 | <50 | <50 | <50 | <50 |
| 17 | S6 | 54.0 | 100.0 | 57.0 | 57.0 | 58.0 | 57.0 | 55.0 | 54.0 | 58.0 | 63.0 | 60.0 |
| 18 | S78 | <50 | 57.0 | 100.0 | 85.0 | 90.0 | 89.0 | 83.0 | 87.0 | 63.0 | 62.0 | 63.0 |
| 18 | S71 | <50 | 57.0 | 85.0 | 100.0 | 95.0 | 96.0 | 77.0 | 79.0 | 61.0 | 64.0 | 67.0 |
| 18 | S7 | <50 | 58.0 | 90.0 | 95.0 | 100.0 | 99.0 | 80.0 | 82.0 | 62.0 | 64.0 | 65.0 |
| 18 | S118 | <50 | 57.0 | 89.0 | 96.0 | 99.0 | 100.0 | 79.0 | 81.0 | 62.0 | 64.0 | 65.0 |
| 18 | S38 | <50 | 55.0 | 83.0 | 77.0 | 80.0 | 79.0 | 100.0 | 89.0 | 64.0 | 60.0 | 63.0 |
| 18 | S82 | <50 | 54.0 | 87.0 | 79.0 | 82.0 | 81.0 | 89.0 | 100.0 | 66.0 | 63.0 | 67.0 |
| 19 | S85 | <50 | 58.0 | 63.0 | 61.0 | 62.0 | 62.0 | 64.0 | 66.0 | 100.0 | 74.0 | 73.0 |
| 20 | S114 | <50 | 63.0 | 62.0 | 64.0 | 64.0 | 64.0 | 60.0 | 63.0 | 74.0 | 100.0 | 80.0 |
| 20 | S119 | <50 | 60.0 | 63.0 | 67.0 | 65.0 | 65.0 | 63.0 | 67.0 | 73.0 | 80.0 | 100.0 |

TABLE 8
Sequence identity among sequences in M23 EAD Family F.

| Group | | S91 | S97 | S11 | S37 | S22 | S107 | S4 | S63 | S35 |
|---|---|---|---|---|---|---|---|---|---|---|
| 21 | S91 | 100.0 | 91.0 | 65.4 | 67.3 | 61.4 | 64.4 | 65.4 | 65.4 | 60.4 |
| 21 | S97 | 91.0 | 100.0 | 69.3 | 70.3 | 62.4 | 66.3 | 67.3 | 67.3 | 63.4 |
| 22 | S11 | 65.4 | 69.3 | 100.0 | 98.0 | 83.0 | 85.0 | 87.0 | 87.0 | 82.0 |
| 22 | S37 | 67.3 | 70.3 | 98.0 | 100.0 | 81.0 | 83.0 | 85.0 | 85.0 | 80.0 |
| 22 | S22 | 61.4 | 62.4 | 83.0 | 81.0 | 100.0 | 83.0 | 84.0 | 84.0 | 86.7 |
| 22 | S107 | 64.4 | 66.3 | 85.0 | 83.0 | 83.0 | 100.0 | 98.0 | 97.0 | 88.0 |
| 22 | S4 | 65.4 | 67.3 | 87.0 | 85.0 | 84.0 | 98.0 | 100.0 | 99.0 | 89.0 |
| 22 | S63 | 65.4 | 67.3 | 87.0 | 85.0 | 84.0 | 97.0 | 99.0 | 100.0 | 89.0 |
| 22 | S35 | 60.4 | 63.4 | 82.0 | 80.0 | 86.7 | 88.0 | 89.0 | 89.0 | 100.0 |

TABLE 9
Sequence identity among sequences in M23 EAD Family G.

| Group | | S27 | S60 | S23 | S104 | S25 | S87 | S16 | S39 |
|---|---|---|---|---|---|---|---|---|---|
| 23 | S27 | 100.0 | 87.3 | 87.3 | 88.2 | 86.3 | 87.3 | 81.4 | 82.4 |
| 23 | S60 | 87.3 | 100.0 | 96.1 | 97.1 | 97.1 | 98.0 | 91.2 | 90.2 |
| 23 | S23 | 87.3 | 96.1 | 100.0 | 99.0 | 97.1 | 98.0 | 89.2 | 91.2 |
| 23 | S104 | 88.2 | 97.1 | 99.0 | 100.0 | 98.0 | 99.0 | 90.2 | 92.2 |
| 23 | S25 | 86.3 | 97.1 | 97.1 | 98.0 | 100.0 | 99.0 | 88.2 | 90.2 |
| 23 | S87 | 87.3 | 98.0 | 98.0 | 99.0 | 99.0 | 100.0 | 89.2 | 91.2 |
| 23 | S16 | 81.4 | 91.2 | 89.2 | 90.2 | 88.2 | 89.2 | 100.0 | 93.1 |
| 23 | S39 | 82.4 | 90.2 | 91.2 | 92.2 | 90.2 | 91.2 | 93.1 | 100.0 |

TABLE 10
Sequence identity among sequences in M23 EAD Family I.

| Group | | S28 | S34 | S73 | S86 | S113 | S65 | S88 | S21 | S79 |
|---|---|---|---|---|---|---|---|---|---|---|
| 30 | S28 | 100.0 | 98.1 | 73.6 | 72.6 | 74.5 | 75.5 | 75.5 | 76.9 | 64.4 |
| 30 | S34 | 98.1 | 100.0 | 72.6 | 71.7 | 73.6 | 74.5 | 74.5 | 76.0 | 63.5 |
| 31 | S73 | 73.6 | 72.6 | 100.0 | 99.0 | 97.2 | 92.5 | 92.5 | 75.5 | 71.7 |
| 31 | S86 | 72.6 | 71.7 | 99.0 | 100.0 | 98.1 | 92.5 | 92.5 | 75.5 | 70.8 |
| 31 | S113 | 74.5 | 73.6 | 97.2 | 98.1 | 100.0 | 94.3 | 94.3 | 77.4 | 70.8 |
| 31 | S65 | 75.5 | 74.5 | 92.5 | 92.5 | 94.3 | 100.0 | 98.1 | 76.4 | 70.8 |
| 31 | S88 | 75.5 | 74.5 | 92.5 | 92.5 | 94.3 | 98.1 | 100.0 | 76.4 | 71.7 |
| 32 | S21 | 76.9 | 76.0 | 75.5 | 75.5 | 77.4 | 76.4 | 76.4 | 100.0 | 75.0 |
| 33 | S79 | 64.4 | 63.5 | 71.7 | 70.8 | 70.8 | 70.8 | 71.7 | 75.0 | 100.0 |

TABLE 11
Sequence identity among sequences in M23 EAD Family J.

| Group | | S20 | S112 |
|---|---|---|---|
| 34 | S20 | 100.0 | 84.5 |
| 34 | S112 | 84.5 | 100.0 |

TABLE 12
Sequence identity among sequences in M23 EAD Family K.

| Group | | S48 | S9 | S44 | S53 | S81 |
|---|---|---|---|---|---|---|
| 35 | S48 | 100.0 | 67.3 | 67.6 | 67.3 | 71.0 |
| 36 | S9 | 67.3 | 100.0 | 97.2 | 92.5 | 90.7 |
| 36 | S44 | 67.6 | 97.2 | 100.0 | 90.7 | 92.5 |
| 36 | S53 | 67.3 | 92.5 | 90.7 | 100.0 | 94.4 |
| 36 | S81 | 71.0 | 90.7 | 92.5 | 94.4 | 100.0 |

In some embodiments, a recombinant protein, e.g., a chimeric CWH, of the disclosure comprises an EAD comprising a sequence motif. In some embodiments, the sequence motif is common to EADs with activity against Staphylococcus. In some embodiments, the sequence motif is common to EADs with activity against Staphylococcus aureus. In some embodiments, the sequence motif is enriched among EADs with activity against Staphylococcus aureus and is not enriched among EADs without activity against Staphylococcus aureus. In some embodiments, the sequence motif is the active M23 EAD motif 1: Y/F M H L/Q S/N K/R Q/R (SEQ ID NO: 147). In some embodiments, the sequence motif is the active M23 EAD motif 2: Y/F/G G G G N/H S/Q/A I/V X I (SEQ ID NO: 148). In some embodiments, the EAD comprises both motif 1 and motif 2. In some embodiments, the recombinant protein comprises i) an EAD comprising active M23 EAD motif 1, and ii) the CBD from LysH5, or a CBD having at least 80, 85, 90, 95, or 99% sequence identity thereto. In some embodiments, the recombinant protein comprises i) an EAD comprising active M23 EAD motif 2, and ii) the CBD from LysH5, or a CBD having at least 80, 85, 90, 95, or 99% sequence identity thereto.

### Recombinant Proteins and Chimeric Proteins of the Disclosure

In some embodiments, the present disclosure provides a recombinant protein comprising the sequence of an EAD according to any one of the embodiments disclosed herein. In some embodiments, the recombinant protein is a chimeric protein. In some embodiments, the chimeric protein is a chimeric cell wall hydrolase (CWH).

A chimeric CWH herein comprises at least one heterologous domain, e.g., a heterologous EAD or CBD, compared to a native CWH sequence. In some embodiments, a chimeric CWH herein is a chimeric protein comprising an EAD and a CBD from different native proteins.

### Enzymatically Active Domains (EADs)

In some embodiments, the chimeric CWH comprises an EAD according to any one of the foregoing embodiments.

In some embodiments, the chimeric CWH comprises 1 EAD. In some embodiments, the CWH comprises more than one EAD. In some embodiments, the chimeric CWH comprises 2 EADs. In some embodiments, the chimeric CWH comprises 3, 4, 5, 6, 7, 8, 9, or 10 EADs.

In some embodiments, the CWH comprises an EAD derived from a lysin. In some embodiments, the lysin is an endolysin, a tail lysin, an exolysin, a bacteriocin, or an autolysin. In some embodiments, the EAD is derived from any one of the endolysins listed herein. In some embodiments, the EAD is a glycosidase. In some embodiments, the EAD is an amidase. In some embodiments, the EAD is a peptidase. In some embodiments, the EAD is a CHAP domain. In some embodiments, the EAD is an M23 domain.

In some embodiments, the EAD is an M23 domain from a bacterial CWH, e.g., Lysostaphin or a related protein. In some embodiments, the EAD is from any one of the proteins listed in Table 13. In some embodiments, the EAD has at least 80, 81, 82, 83, 84, 85, 86, 87, 88, 89, 90, 91, 92, 93, 94, 95, 96, 97, 98, or 99% identity with an EAD from any one of the proteins listed in Table 13. In some embodiments, the EAD differs from an EAD from any one of the proteins listed in Table 13 by 1, 2, 3, 4, 5, 6, 7, 8, 9, 10, 11, 12, 13, 14, or 15 amino acids.

TABLE 13
Illustrative bacterial cell wall hydrolases.

| Gene Name | Organism | GenBank Accession No. |
|---|---|---|
| Lss (Lysostaphin) | Staphylococcus simulans | M15686.1 |
| ALE-1 | Staphylococcus capitis | D86328.1 |
| LytM | Staphylococcus aureus | L77194.1 |
| LytU | Staphylococcus aureus | BAB41427.1 |
| SpM23_A | Staphylococcus pettenkoferi | WP_049408311.1 |
| SpM23_B | Staphylococcus pettenkoferi | ASE36562.1 |

In some embodiments, the EAD is the Lss EAD (SEQ ID NO: 152). In some embodiments, the EAD has at least 80, 81, 82, 83, 84, 85, 86, 87, 88, 89, 90, 91, 92, 93, 94, 95, 96, 97, 98, or 99% identity with the Lss EAD (SEQ ID NO: 152). In some embodiments, the EAD differs from the Lss EAD (SEQ ID NO: 152) by 1, 2, 3, 4, 5, 6, 7, 8, 9, 10, 11, 12, 13, 14, or 15 amino acids.

In some embodiments, the EAD is the ALE-1 EAD (SEQ ID NO: 153). In some embodiments, the EAD has at least 80, 81, 82, 83, 84, 85, 86, 87, 88, 89, 90, 91, 92, 93, 94, 95, 96, 97, 98, or 99% identity with the ALE-1 EAD (SEQ ID NO: 153). In some embodiments, the EAD differs from the ALE-1 EAD (SEQ ID NO: 153) by 1, 2, 3, 4, 5, 6, 7, 8, 9, 10, 11, 12, 13, 14, or 15 amino acids.

In some embodiments, the EAD is the LytM EAD (SEQ ID NO: 154). In some embodiments, the EAD has at least 80, 81, 82, 83, 84, 85, 86, 87, 88, 89, 90, 91, 92, 93, 94, 95, 96, 97, 98, or 99% identity with the LytM EAD (SEQ ID NO: 154). In some embodiments, the EAD differs from the LytM EAD (SEQ ID NO: 154) by 1, 2, 3, 4, 5, 6, 7, 8, 9, 10, 11, 12, 13, 14, or 15 amino acids.

### Cell Wall Binding Domains (CBDs)

In some embodiments, the chimeric CWH comprises a cell wall binding domain (CBD). In some embodiments, the CBD is derived from a Staphylococcus phage protein. In some embodiments, the CBD is derived from a Staphylococcus phage CWH. In some embodiments, the CBD is derived from a Staphylococcus phage lysin. In some embodiments, the lysin is an endolysin, a tail lysin, an exolysin, a bacteriocin, or an autolysin. In some embodiments, the CBD is an SH3b domain.

In some embodiments, the CBD is derived from an endolysin. In some embodiments, the CBD has at least 80, 81, 82, 83, 84, 85, 86, 87, 88, 89, 90, 91, 92, 93, 94, 95, 96, 97, 98, or 99% identity to a CBD from an endolysin. In some embodiments, the endolysin is any one of the following endolysins: LysPALS1 (derived from Staphylococcus aureus phage PALS1), PlySs2 (derived from Staphylococcus saprophyticus phage Ss2), LysK (derived from Staphylococcus aureus phage K), LysGH15 (derived from Staphylococcus epidermidis phage GH15), LysH5 (derived from Staphylococcus aureus phage H5), LysK2 (derived from Staphylococcus aureus phage K2), LysSA97 (derived from Staphylococcus aureus phage SA97), LysH28S (derived from Staphylococcus aureus phage H28S), LysP (derived from Staphylococcus aureus phage P68), LysSA1l (derived from Staphylococcus aureus phage SA11), LysSA012 (derived from Staphylococcus aureus phage SA012), LysSAP-2 (derived from Staphylococcus aureus phage SAP-2.), LysGH15B (derived from Staphylococcus epidermidis phage GH15B), LysSb01 (derived from Staphylococcus aureus phage Sb01), LysSAP-3 (derived from Staphylococcus aureus phage SAP-3), LysSA012-1 (derived from Staphylococcus aureus phage SA012-1), LysKUMC-1 (derived from Staphylococcus aureus phage KUMC-1), LysSAP-4 (derived from Staphylococcus aureus phage SAP-4), LysSA11-1 (derived from Staphylococcus aureus phage SA11-1), LysGH15C (derived from Staphylococcus epidermidis phage GH15C), LysF1 (derived from Staphylococcus aureus phage F1), LysH1 (derived from Staphylococcus aureus phage H1.), LysSAP-5 (derived from Staphylococcus aureus phage SAP-5), LysSAP-6 (derived from Staphylococcus aureus phage SAP-6), LysSA11-2 (derived from Staphylococcus aureus phage SA11-2), LysK-J47 (derived from Staphylococcus aureus phage J47), LysSA11-3 (derived from Staphylococcus aureus phage SA11-3), LysF11 (derived from Staphylococcus aureus phage F11), LysA72 (derived from Staphylococcus aureus phage A72), LysKAK14 (derived from Staphylococcus aureus phage KAK14), LysSAP-7 (derived from Staphylococcus aureus phage SAP-7), LysGH15D (derived from Staphylococcus epidermidis phage GH15D.), LysK-VA-20 (derived from Staphylococcus aureus phage VA-20), LysGH15E (derived from Staphylococcus epidermidis phage GH15E), LysPVL (derived from Staphylococcus aureus phage PVL), LysGIMI (derived from Staphylococcus aureus phage GIM1), LysGH15F (derived from Staphylococcus epidermidis phage GH15F), LysGH15G (derived from Staphylococcus epidermidis phage GH15G), LysSA012-2 (derived from Staphylococcus aureus phage SA012-2), LysA72-2 (derived from Staphylococcus aureus phage A72-2), LysSAP-8 (derived from Staphylococcus aureus phage SAP-8), and LysSAP-9 (derived from Staphylococcus aureus phage SAP-9).

In some embodiments, the CBD is derived from a bacterial protein with activity against Staphylococcus, e.g., a bacteriocin. In some embodiments, the CBD has at least 80, 81, 82, 83, 84, 85, 86, 87, 88, 89, 90, 91, 92, 93, 94, 95, 96, 97, 98, or 99% identity to a CBD from a bacteriocin. In some embodiments, the bacteriocin is any one of the following bacteriocins: ALE-1 (produced by Staphylococcus simulans), Lysostaphin (produced by Staphylococcus simulans), SpM23_A (produced by Sphingomonas paucimobilis), SpM23_B (produced by Sphingomonas paucimobilis), Enterocin (produced by Enterococcus faecium), Staphylococcin C55 (produced by Staphylococcus epidermidis), Plantaricin (produced by Lactobacillus plantarum), Staphylococcin G (produced by Staphylococcus simulans), BacST33LD (produced by Bacillus licheniformis), Lacticin 3147 (produced by Lactococcus lactis), Aureocin A70 (produced by Staphylococcus aureus), Nisin (produced by Lactococcus lactis), Pediocin (produced by Pediococcus acidilactici), Lactococcin (produced by Lactococcus lactis), Bacillocin (produced by Bacillus subtilis), Amylocyclicin (Bacillus amyloliquefaciens), Thuricin (produced by Bacillus thuringiensis), Epidermin (produced by Staphylococcus epidermidis), Staphylococcin: produced by Staphylococcus hyicus), Aureocin A53 (produced by Staphylococcus aureus), Staphylococcin C55 (produced by Staphylococcus cohnii), Staphylococcin G (produced by Staphylococcus epidermidis), Staphylococcin P (produced by Staphylococcus epidermidis), Staphylococcin S (produced by Staphylococcus aureus), Staphylococcin T (produced by Staphylococcus hyicus), Staphylococcin U (produced by Staphylococcus saprophyticus), Staphylococcin V (produced by Staphylococcus sciuri), Bacilysin (produced by Bacillus amyloliquefaciens), Carnocyclin A (produced by Carnobacterium maltaromaticum), Curvacin (produced by Lactobacillus curvatus), Garvicin (produced by Lactococcus garvieae), Lacticin (produced by Lactococcus lactis), Leucocin (produced by Leuconostoc mesenteroides), Piscicolin (produced by Carnobacterium piscicola), and Staphylococcin 188 (produced by Staphylococcus aureus).

In some embodiments, the CBD is derived from a bacterial protein with activity against Staphylococcus, e.g., an autolysin. In some embodiments, the CBD has at least 80, 81, 82, 83, 84, 85, 86, 87, 88, 89, 90, 91, 92, 93, 94, 95, 96, 97, 98, or 99% identity to a CBD from an autolysin. In some embodiments, the autolysin is any one of the following autolysins: AtlA, AtlE, AtIS, LytA, LytB, LytC, LytD, LytF, LytG, LytH, LytM, LytN, LytP, LytR, LytS, LytT, LytU, LytV, LytX, LytY, LytZ, N-acetylmuramoyl-L-alanine amidase, N-acetylmuramoyl-L-alanine amidase domain protein, OatA, PcsB, SceD, Sle1, Sle2, Sle3, Sle4, Aaa, Alp, Ami, Atl, CwlH, Epi, IsaA, LysK, N-acetylmuramoyl-L-alanine amidase 2, OatB, SagB, SsaA, SsaB, SsaC, SsaD, SsaE, SsaF, SsaG, SsaH, Ssal, SsaJ, SsaK, SsaL, SsaM, SsaN, SsaO, SsaP, SsaQ, SsaR, SsaS, SsaT, SsaU, SsaV, SsaW, SsaX, SsaY, SsaZ, AcmA, LytE, and AaeA.

In some embodiments, the chimeric CWH comprises a CBD having at least 80, 81, 82, 83, 84, 85, 86, 87, 88, 89, 90, 91, 92, 93, 94, 95, 96, 97, 98, or 99% identity to the CBD from ALE-1 (SEQ ID NO: 127), LysA72 (SEQ ID NO: 125), LysH5 (SEQ ID NO: 124), LysPALS1 (SEQ ID NO: 129), LysSA97 (SEQ ID NO: 128), or PlySs2 (SEQ ID NO: 126). In some embodiments, the chimeric CWH comprises the CBD from ALE-1 (SEQ ID NO: 127), LysA72 (SEQ ID NO: 125), LysH5 (SEQ ID NO: 124), LysPALS1 (SEQ ID NO: 129), LysSA97 (SEQ ID NO: 128), or PlySs2 (SEQ ID NO: 126). In some embodiments, the chimeric protein comprises a CBD having the sequence of, or a sequence having at least 80, 81, 82, 83, 84, 85, 86, 87, 88, 89, 90, 91, 92, 93, 94, 95, 96, 97, 98, or 99% identity to the sequence of, a CBD listed in Table 14 below. In some embodiments, the chimeric CWH comprises a CBD having at least 80, 81, 82, 83, 84, 85, 86, 87, 88, 89, 90, 91, 92, 93, 94, 95, 96, 97, 98, or 99% identity to the CBD from LysH5 (SEQ ID NO: 124). In some embodiments, the chimeric CWH comprises the CBD from LysH5 (SEQ ID NO: 124).

TABLE 14
Illustrative CBD sequences of the disclosure.

| CBD | SEQ | |
|---|---|---|
| Description | ID NO | Sequence |
| LysH5 CBD | 124 | SNDSSASSNTVKPVASAWKRNKYGTY |
| | | YMEESARFTNGNQPITVRKVGPFLS |
| | | CPVGYQFQPGGYCDYTEVMLQDGHV |
| | | WVGYTWEGQRYYLPIRTWNGSAPPN |
| | | QILGDLWGEIS |
| LysA72 CBD | 125 | KNPPVPAGYTLDKNNVPYKKEAGNY |
| | | TVANVKGNNVRDGYSTNSRITGVLP |
| | | NNATIKYDGAYCINGYRWITYIANS |
| | | GQRRYIATGEVDKAGNRISSFGKFS |
| | | TI |
| PlySs2 CBD | 126 | SRSYRETGTMTVTVDALNVRRAPNT |
| | | SGEIVAVYKRGESFDYDTVIIDVNG |
| | | YVWVSYIGGSGKRNYVATGATKDGK |
| | | RFGNAWGTFK |
| ALE-1 CBD | 127 | MPFLKSAGYGSNSTSSSNNNGYKTN |
| | | KYGTLYKSESASFTANTDIITRLTG |
| | | PFRSMPQSGVLRKGLTIKYDEVMKQ |
| | | DGHVWVGYNTNSGKRVYLPVRTWNE |
| | | STGELGPLWGTIK |
| LysSA97 CBD | 128 | PSSKPSADKITWNWKGVFYPNPEKA |
| | | IRVRKTAGLTGTVVEEDSWLYTKDD |
| | | WVKFDQVIKKDGYWWIRFKYQREGS |
| | | STNNFYCAVCRITDKEQKIKNEKYW |
| | | GTIEWA |
| LysPALS1 CBD | 129 | SASTPATRPVTGSWKKNQYGTWYKP |
| | | ESATFVNGNQPIVTRIGSPFLNAPV |
| | | GGNLPAGATIVYDEVCIQAGHIWIG |
| | | YNAYNGNRVYCPVRTCQGVPPSHVP |
| | | GVAWGTFK |

### Linkers

In some embodiments, a chimeric protein herein comprises more than one domain, and the domains are joined by a linker. In some embodiments, the linker is a flexible linker. In some embodiments, the linker is an amino acid sequence between 1-100 amino acids in length, including all values and subranges therebetween. In some embodiments, the linker comprises one or more glycines and/or serines.

### Protein Tags

In some embodiments, a recombinant protein of the disclosure comprises a protein tag. A protein tag is typically a short sequence of amino acids, or a protein domain, that is fused to a recombinant protein in order to facilitate purification and/or visualization. In some embodiments, a protein tag improves protein solubility. In some embodiments, the tag is a His tag, a GST tag, an MBP tag, a Strep tag, a FLAG tag, a GFP tag, an HA tag, a V5 tag, an Avi tag, a CBP tag, a ZZ tag, a SUMO tag, an Fc tag, a Thioredoxin tag, a Protein kinase A (PKA) tag, a Myc tag, or an S tag, or any combination thereof. In some embodiments, the tag is a His tag and comprises 6 histidine residues.

### Nucleic Acids, Vectors, and Host Cells of the Disclosure

The present disclosure also provides nucleic acids encoding the EADs of the disclosure and the chimeric proteins, e.g., CWHs, of the disclosure. The present disclosure also provides vectors and host cells for expression of the EADs and chimeric proteins of the disclosure. In some embodiments, the vector is a plasmid, a cosmid, a bacteriophage, or a virus comprising a nucleic acid of the disclosure. In some embodiments, the host cell comprises a nucleic acid of the disclosure or a vector of the disclosure. In some embodiments, the host cell is a bacterial cell, a yeast cell, an insect cell, a mammalian cell, or a plant cell.

### Formulations of the Disclosure

The present disclosure provides compositions comprising the novel EADs, chimeric proteins (e.g., CWHs), nucleic acids, vectors, or host cells disclosed herein. In some embodiments, these compositions are formulated for delivery to a subject for the treatment of a condition associated with Staphylococcus sp.

### Topical, Parenteral, and Enteral Formulations

In some embodiments, compositions of the disclosure are formulated for topical, parenteral, or enteral administration.

In some embodiments, a composition herein is formulated for topical administration. Formulations for topical administration include lotions, hydrogels, creams, ointments, gels, drops, transdermal patches, colloidal patches, powders, suppositories, sprays, liquids, semi-solids, monophasic compositions, multiphasic compositions (e.g., oil-in-water, water-in-oil), foams, microsponges, liposomes, nanoemulsions, aerosol foams, polymers, fullerenes, and powders. In some embodiments, carriers, bases, thickeners, penetration enhancers, buffers, diluents, emulsifiers, humectants, dispersing aids, binders, and/or excipients are added to the formulation. In some embodiments, the composition is formulated as a hydrogel. In some embodiments, the composition is formulated as a lotion. In some embodiments, the composition is formulated as a cream. In some embodiments, the composition is formulated as a freeze dried powder, e.g., which can be reconstituted with liquid prior to use. In some embodiments, the composition is a colloidal patch.

In some embodiments, the compositions of the disclosure are formulated for parenteral administration. As used herein, “parenteral administration” of a composition includes any route of administration characterized by physical breaching of a tissue of a subject and administration of the composition through the breach in the tissue, thus generally resulting in the direct administration into the blood stream, into muscle, or into an internal organ. Parenteral administration thus includes, but is not limited to, administration of a composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, intrasynovial injection or infusions; and kidney dialytic infusion techniques.

In some embodiments, a composition herein is prepared for oral administration. The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal. The compositions of the present disclosure may be formulated into any of many possible dosage forms such as, but not limited to, tablets, capsules, liquid syrups, soft gels, suppositories, aerosols, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

### Formulation Ingredients

In some embodiments, the composition comprises an emulsifier. In some embodiments, the composition comprises a mixture of emulsifiers.

In some embodiments, the composition comprises about 0.5% to about 5% w/v of an emulsifier or a mixture of emulsifiers.

Examples of emulsifiers suitable for use in some embodiments of the disclosure include xanthan gum, polysorbate 80, oleoyl polyoxyl-6 glycerides, polyoxyl 35 hydrogenated castor oil, sucrose distearate, saponin, sodium alginate, guar gum, tocopherol polyethylene glycol 1000 succinate, lauroyl polyoxyl-32 glycerides, sorbitan monooleate, glyceryl stearate, cetearyl alcohol, sodium stearoyl lactylate, salts thereof, derivatives thereof, and mixtures thereof. In some embodiments, the emulsifier is xanthan gum.

In some embodiments, emulsifier components are selected from poly-glycolized glycerides and polyoxyethylene glycerides of medium to long chain mono-, di-, and triglycerides, such as: almond oil PEG-6 esters, almond oil PEG-60 esters, apricot kernel oil PEG-6 esters (Labrafil® M1944CS), caprylic/capric triglycerides PEG-4 esters (Labrafac® Hydro WL 1219), caprylic/capric triglycerides PEG-4 complex (Labrafac® Hydrophile), caprylic/capric glycerides PEG-6 esters (Softigen® 767), caprylic/capric glycerides PEG-8 esters (Labrasol®), castor oil PEG-50 esters, hydrogenated castor oil PEG-5 esters, hydrogenated castor oil PEG-7 esters, 9 hydrogenated castor oil PEG-9 esters, corn oil PEG-6 esters (Labrafil® M 2125 CS), corn oil PEG-8 esters (Labrafil® WL 2609 BS), corn glycerides PEG-60 esters, olive oil PEG-6 esters (Labrafil® M1980 CS), hydrogenated palm/palm kernel oil PEG-6 esters (Labrafil® M 2130 BS), hydrogenated palm/palm kernel oil PEG-6 esters with palm kernel oil, PEG-6, palm oil (Labrafil® M 2130 CS), palm kernel oil PEG-40 esters, peanut oil PEG-6 esters (Labrafil® M 1969 CS), glycerol esters of saturated C8-C18 fatty acids (Gelucire® 33/01), glyceryl esters of saturated C12-C18 fatty acids (Gelucire® 39/01 and 43/01), glyceryl laurate/PEG-32 laurate (Gelucire® 44/14), glyceryl laurate glyceryl/PEG 20 laurate, glyceryl laurate glyceryl/PEG 32 laurate, glyceryl, laurate glyceryl/PEG 40 laurate, glyceryl oleate/PEG-20 glyceryl, glyceryl oleate/PEG-30 oleate, glyceryl palmitostearate/PEG-32 palmitostearate (Gelucire® 50/13), glyceryl stearate/PEG stearate, glyceryl stearate/PEG-32 stearate (Gelucire® 53/10), saturated polyglycolized glycerides (Gelucire® 37/02 and Gelucire® 50/02), triisostearin PEG-6 esters (i.e. Labrafil® Isostearique), triolein PEG-6 esters, trioleate PEG-25 esters, polyoxyl 35 castor oil (Cremophor® EL or Kolliphor® EL), polyoxyl 40 hydrogenated castor oil (Cremophor® RH 40 or Kolliphor® RH40), polyoxyl 60 hydrogenated castor oil (Cremophor® RH60), lecithin, phospholipids and mixtures thereof.

In some embodiments, the emulsifier is polyglycolized derivatives and polyoxyethylene esters or ethers derivatives of medium to long chain fatty acids, commercially named Brij and Myrj variety surfactants, and propylene glycol esters of medium to long chain fatty acids, which can be used including caprylate/caprate diglycerides, glyceryl monooleate, glyceryl ricinoleate, glyceryl laurate, glyceryl dilaurate, glyceryl dioleate, glyceryl mono/dioleate, glyceryl caprylate/caprate, medium chain (C8/C10) mono- and diglycerides (Capmul® MCM, Capmul® MCM (L)), mono- and diacetylated monoglycerides, polyglyceryl oleate, polyglyceryl-2 dioleate, polyglyceryl-10 trioleate, polyglyceryl-10 laurate, polyglyceryl-10 oleate, and polyglyceryl-10 mono dioleate, propylene glycol caprylate/caprate (Labrafac® PC), propylene glycol dicaprylate/dicaprate (Miglyol® 840), propylene glycol monolaurate, propylene glycol ricinoleate, propylene glycol monooleate, propylene glycol dicaprylate/dicaprate, propylene glycol dioctanoate, and mixtures thereof.

In some embodiments, the composition comprises a humectant. In some embodiments, the composition is a topical formulation and comprises a humectant, which can be referred to as a soothing, smoothing, moisturizing, or protective agent. Humectants of the present disclosure function to stabilize the moisture content of the tissue to which it is applied in the presence of fluctuating humidity.

In some embodiments, the humectant is selected from: polyglycols (as hereinafter defined), propylene glycol, sorbitol, lactic acid, sodium lactate, glycerol, glycerine, ethoxylated castor oil, calamine, dodecylsulphate, sodium lauryl sulphate (SLS); a polyoxyethylene ester of polysorbitan, such as monooleate, monolaurate, monopalmitate, monostearate esters; esters of sorbitan, the polyoxyethylenes ethers, sodium dioctylsulphosuccinate (DOSS), lecithin, sodium docusate, hexylene glycol, butylene glycol, aloe vera gel, aloe vera powder, hyaluronic acid, alpha hydroxy acids such as lactic acid, egg yolk, egg white, glyceryl triacetate, honey, molasses, polymeric polyols such as polydextrose, quillaia, sodium hexametaphosphate e452i; sugar alcohols (sugar polyols) such as glycerol, sorbitol, xylitol, maltitol; urea, and castor oil.

In some embodiments, the composition comprises a humectant selected from the list consisting of: aloe vera, betaine, butylene glycol, caprylyl glycol, dimethicone, fructose, glucomannan, glucose, glycerin, glyceryl glucoside, honey, hyaluronic acid, lactic acid, panthenol, polyethylene glycol, propylene glycol, propanediol, sodium hyaluronate, sodium lactate, sodium pyrrolidone carboxylic acid, sorbitol, and urea. In some embodiments, the composition comprises 0.1-50% w/v humectant, including all values and subranges therebetween. In some embodiments, the composition comprises 0.5-10% w/v humectant.

In some embodiments, the composition comprises hyaluronic acid. In some embodiments, the composition is a hyaluronic-based hydrogel for topical application. In some embodiments, the composition comprises 0.1-10% w/v hyaluronic acid. In some embodiments, the composition comprises 0.5-5.0% w/v hyaluronic acid. In some embodiments, the composition comprises 1-2% w/v hyaluronic acid. In some embodiments, the composition comprises a hydrogel. In some embodiments, the hydrogel comprises a cellulose polymer. In some embodiments the hydrogel comprises hydroxypropyl methylcellulose.

In some embodiments, the composition comprises a cellulose polymer. In some embodiments, the cellulose polymer is hydroxyethyl cellulose, methylcellulose, hydroxy methylcellulose, carboxymethyl cellulose, microcrystalline cellulose, ethyl cellulose, sodium carboxymethyl cellulose, hydroxypropyl cellulose, or cellulose acetate. In some embodiments, the composition comprises 0.1-20% w/v cellulose polymer, including all values and subranges therebetween. In some embodiments, the composition comprises 0.5-10% w/v of a cellulose polymer. In some embodiments, the composition comprises 1-5% w/v of a cellulose polymer.

In some embodiments, the composition comprises a thickening agent, a gelling agent, and/or a polymer. In some embodiments, the composition comprises an acrylate. In some embodiments, the composition comprises a carbomer.

In some embodiments, the composition comprises a salt. In some embodiments, the composition comprises a salt selected from the list consisting of: calcium chloride, Dead Sea salt, Epsom salt, Himalayan pink salt, magnesium chloride, sea salt, and sodium chloride. In some embodiments, the composition comprises 10-500 mM of a salt, including all values and subranges therebetween. In some embodiments, the composition comprises 50-250 mM of a salt.

In some embodiments, the composition comprises a buffer. In some embodiments, the buffer is 4-(2-hydroxyethyl)-1-piperazineethanesulfonic acid, acetic acid, ammonium acetate, boric acid, citric acid, glycine, phosphoric acid, potassium hydroxide, potassium phosphate, sodium acetate, sodium bicarbonate, sodium borate, sodium carbonate, sodium citrate, sodium dihydrogen phosphate, sodium hydrogen phosphate, sodium hydroxide, sodium phosphate, sodium tetraborate, tris(hydroxymethyl)aminomethane, or trisodium phosphate. In some embodiments, the composition comprises 1-250 mM of a buffer, including all values and subranges therebetween. In some embodiments, the composition comprises 5-50 mM of a buffer.

In some embodiments, the composition comprises a surfactant. In some embodiments, the composition comprises a surfactant selected from the list consisting of: ceteareth-20, cocamidopropyl betaine, coco-glucoside, decyl glucoside, decyl polyglucose, disodium laureth sulfosuccinate, glycereth-26, lauryl glucoside, lauryl polyglucose, sodium cocoyl glutamate, sodium cocoyl isethionate, sodium laureth sulfate, and sodium lauryl sulfate. In some embodiments, the composition comprises 0.1-20% w/v of a surfactant, including all values and subranges therebetween. In some embodiments, the composition comprises 1-10% w/v of a surfactant.

In some embodiments, the composition comprises an oil. In some embodiments, the composition comprises an oil selected from the list consisting of: argan oil, avocado oil, baobab oil, camellia oil, carrot seed oil, coconut oil, evening primrose oil, grapeseed oil, hemp seed oil, jojoba oil, macadamia nut oil, marula oil, mineral oil, olive oil, pomegranate seed oil, raspberry seed oil, rosehip seed oil, squalane oil, sunflower seed oil, sweet almond oil, and tamanu oil. In some embodiments, the composition comprises 0.1-20% w/v of an oil, including all values and subranges therebetween.

In some embodiments, the composition comprises an alcohol. In some embodiments, the composition comprises an alcohol selected from the list consisting of: cetyl alcohol, ethyl alcohol, isopropyl alcohol, and stearyl alcohol. In some embodiments, the composition comprises 0.1-20% w/v of an alcohol, including all values and subranges therebetween. In some embodiments, the composition comprises 1-10% w/v of an alcohol.

In some embodiments, the composition comprises a free amino acid. In some embodiments, the composition comprises alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some embodiments, the composition comprises an amino acid selected from the list consisting of: alanine, arginine, cysteine, glutamine, glycine, histidine, lysine, methionine, proline, serine, and threonine. In some embodiments, the composition comprises 10-250 mM of an amino acid, including all values and subranges therebetween. In some embodiments, the composition comprises 25-150 mM of an amino acid.

In some embodiments, the composition comprises glycerol. In some embodiments, the composition comprises 0.5-50% w/v glycerol, including all values and subranges therebetween. In some embodiments, the composition comprises 1-30% w/v glycerol. In some embodiments, the composition comprises 1-5% w/v glycerol.

In some embodiments, the composition comprises petrolatum. In some embodiments, the composition comprises 0.1-20% w/v petrolatum, including all values and subranges therebetween.

The compositions of the present disclosure can comprise an additional agent or agents, whether active or passive. Examples of such an agent include a sweetening agent, a flavoring agent, a coloring agent, a filling agent, a binding agent, a lubricating agent, an excipient, a preservative, an emollient, a hydrating agent, a smoothing agent, or a manufacturing agent. Additional excipients or additives can be added to the composition. For example, if desired, any generally accepted soluble or insoluble inert filler (diluent) material can be included in the final product (e.g., a solid dosage form). Such inert filler can comprise a monosaccharide, a disaccharide, a polyhydric alcohol, inorganic phosphates, sulfates or carbonates, and combinations thereof. Examples of suitable inert fillers include sucrose, dextrose, lactose, xylitol, fructose, sorbitol, calcium phosphate, calcium sulfate, calcium carbonate, microcrystalline cellulose, and combinations thereof. An effective amount of any generally accepted lubricant, such as calcium or magnesium soaps, can be added.

Depending on the dosage form, optional additives and modifiers further comprise one or more of acids, bases, acidity regulators, alcohol, anticaking agents, antifoaming agents, antioxidants, bulking agents, coagulation agents, colour retention agents, emulsifiers, flavor enhancers, flour treatment agents, gelling agents, glazing agents, humectants, leavening agents, tracer gases, preservatives, stabilizers, sweeteners, tenderizers, and thickeners.

The compositions of the present disclosure may additionally contain other conventional adjunct components. Thus, for example, the compositions may contain additional, compatible, active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

In some embodiments, the composition comprises a skin protectant. In some embodiments, the composition comprises an ingredient that is FDA-approved for the treatment of atopic dermatitis. In some embodiments, the composition comprises an FDA-approved skin protectant. In some embodiments, the composition comprises colloidal oatmeal. In some embodiments, the composition comprises a skin protectant selected from the list consisting of: allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, white petrolatum, zinc acetate, zinc carbonate, and zinc oxide. In some embodiments, the composition comprises any one of the following skin protectants in the following ranges: allantoin, 0.5 to 2%; aluminum hydroxide gel, 0.15 to 5%; calamine, 1 to 25%; cocoa butter, 50 to 100%; cod liver oil, 5 to 13.56%; colloidal oatmeal, 0.007% minimum, or 0.003% minimum in combination with mineral oil; dimethicone, 1 to 30%; glycerin, 20 to 45%; hard fat, 50 to 100%; kaolin, 4 to 20%; lanolin, 12.5 to 50%; mineral oil, 50 to 100%, or 30 to 35% in combination with colloidal oatmeal; petrolatum, 30 to 100%; sodium bicarbonate; topical starch, 10 to 98%; white petrolatum, 30 to 100%; zinc acetate, 0.1 to 2%; zinc carbonate, 0.2 to 2%; zinc oxide, 1 to 25%. See, e.g., Sec. 347.10 of CFR Title 21, Volume 5, “Skin protectant active ingredients,” incorporated by reference herein in its entirety.

In some embodiments, other ingredients are also present in the composition, such as antibiotics; antiseptics; antifungals; corticosteroids; soothing agents; anti-aging agents; smoothing agents; moisturizing agents; and protective agents.

### Characteristics of Compositions of the Disclosure

The present disclosure provides EADs and chimeric proteins comprising those EADs, as well as compositions comprising these EADs and/or chimeric proteins. These compositions have beneficial characteristics for therapeutic use against target Staphylococcus sp.

In addition to issues with selectivity, prior CWHs and endolysins known in the art have properties that are not well-suited for therapeutic indications, e.g., topical application. For example, many previously characterized CWHs suffer from weak activity, low thermostability, and/or narrow or unsuitable pH range.

The present disclosure provides EADs and chimeric proteins comprising those EADs with beneficial characteristics, such as, but not limited to, high anti-Staphylococcus activity, Staphylococcus species specificity, thermostability, and broader or more suitable pH range. In some embodiments, the present disclosure provides combination compositions that exhibit synergistic anti-Staphylococcus activity.

### Anti-Staphylococcus Activity

The compositions of the present disclosure are active against Staphylococcus species, e.g., a target Staphylococcus species.

In some embodiments, a composition of the disclosure has activity against a target Staphylococcus species and the degree of that activity is determined based on its MIC against that target species. In some embodiments, the MIC is less than 100, 90, 80, 70, 60, 50, 40, 30, 20, or 10 μg/mL. In some embodiments, the MIC is less than 20, 19, 18, 17, 16, 15, 14, 13, 12, 11, 10, 9, 8, 7, 6, 5, 4, 3, 2, or 1 μg/mL. In some embodiments, the MIC is less than 5.0, 4.5, 4.0, 3.5, 3.0, 2.5, 2.0, 1.5, 1.0, or 0.5 μg/mL. In some embodiments, the MIC is less than 1.0, 0.9, 0.8, 0.7, 0.6, 0.5, 0.4, 0.3, 0.2, or 0.1 μg/mL.

The genus Staphylococcus is composed of Gram-positive and facultative anaerobic bacteria present in cutaneous and mucous membrane microbiota of mammals and birds. Staphylococcus is a genus comprised of nonspore-forming cocci belonging to the family Micrococcaceae. Staphylococcus sp. are often found as normal human microbiota of the skin and nasal cavity. The genus includes clinically relevant opportunistic pathogens in both human and veterinary medicine. The species belonging to this genus can be grouped and differentiated according to the production of the enzyme coagulase, which is an enzyme capable of converting fibrinogen into fibrin, a characteristic that is easily detectable in the laboratory and allows for a practical classification.

In general, coagulase-positive staphylococci (CoPS), such as S. aureus, S. intermedius and S. pseudointermedius, among others, are usually pathogenic, even though in some cases they can cause asymptomatic colonization in healthy individuals. In some embodiments, a composition of the disclosure is active against a coagulase-positive staphylococci (CoPS) species. In some embodiments, the species is S. aureus. In some embodiments, the compositions of the present disclosure are active against the species S. intermedius. In some embodiments, the compositions of the present disclosure are active against the species S. pseudointermedius.

Coagulase-negative staphylococci (CoNS), represented by a larger group of species, have been associated with opportunistic infections. CONS species, such as S. epidermidis, S. haemolyticus and S. lugdunensis have been associated with opportunistic infections in humans. S. epidermidis and some subspecies of S. schleiferi can cause skin and ear infections in dogs. S. felis can cause lower urinary tract disease, eye infections, and otitis in cats. In some embodiments, a composition of the disclosure is active against a coagulase-negative staphylococci (CoNS) species.

In some embodiments, a composition of the disclosure is selective for one or more CoPS species in comparison to one or more CoNS species.

In some embodiments, the compositions of the present disclosure are active against S. aureus. S. aureus is considered the most important pathogen of the genus. In people, it can be found in community settings and hospital premises, constituting an important source of infections associated with healthcare. The bacterium can produce infections in humans associated with skin and soft tissue, pneumonia, septicemia and osteomyelitis. These conditions have also been reported in animals. S. aureus is able to cause many superficial pyogenic (pus-forming) infections of the dermis and underlying tissues as well as serious systemic infections. It can produce a range of toxins including enterotoxins (food poisoning), cytotoxins (general systemic toxins), and toxic shock superantigens.

In some embodiments, the compositions of the present disclosure are active against S. pseudointermedius. S. pseudointermedius is a common cause of skin and soft tissue infections in humans, as well as in dogs and cats.

In some embodiments, the compositions of the present disclosure are active against S. epidermidis. S. epidermidis is a common cause of infections associated with medical devices such as catheters, pacemakers, and prosthetic joints. It is also a leading cause of bloodstream infections in hospitalized patients.

In some embodiments, the compositions of the present disclosure are active against S. saprophyticus. S. saprophyticus is a common cause of urinary tract infections in young, sexually active women.

In some embodiments, the compositions of the present disclosure are active against S. haemolyticus. S. haemolyticus is a leading cause of infections associated with central venous catheters and other medical devices, particularly in immunocompromised patients.

In some embodiments, the compositions of the present disclosure are active against S. lugdunensis. S. lugdunensis is increasingly recognized as a significant pathogen, particularly in skin and soft tissue infections, endocarditis, and bone and joint infections.

In some embodiments, a composition of the disclosure is active against a species of Staphylococcus. In some embodiments, a composition of the disclosure is active against a Staphylococcus species selected from: S. agnetis, S. argensis, S. argenteus, S. arlettae, S. aureus, S. auricularis, S. capitis, S. caprae, S. carnosus, S. chromogenes, S. cohnii, S. condimenti, S. cornubiensis, S. delphini, S. devriesei, S. edaphicus, S. epidermidis, S. equi, S. equorum, S. felis, S. fleurettii, S. gallinarum, S. haemolyticus, S. hominis, S. hyicus, S. intermedius, S. kloosii, S. lentus, S. lugdunensis, S. lutrae, S. massiliensis, S. microti, S. muscae, S. nepalensis, S. pasteuri, S. petrasii, S. pettenkoferi, S. piscifermentans, S. pseudintermedius, S. pseudoxylosus, S. rostri, S. saccharolyticus, S. saprophyticus, S. schleiferi, S. schweitzeri, S. sciuri, S. simiae, S. simulans, S. stepanovicii, S. succinus, S. vitulinus, S. warneri, and S. xylosus.

In some embodiments, a composition of the disclosure is active against a Staphylococcus species in the same phylogenetic grouping as S. aureus. In some embodiments, a composition of the disclosure is active against S. argenteus, S. aureus, S. schweitzeri, or S. simiae. See, e.g., Madhaiyan et al., Int. J. Syst. Evol. Microbiol 2020; 70:5926-5936, incorporated by reference herein, which provides a phylogenetic analysis of Staphylococcus species.

In some embodiments, a composition of the disclosure is active against a Staphylococcus species in the same phylogenetic grouping as S. epidermidis. In some embodiments, a composition of the disclosure is active against S. capitis, S. caprae, S. epidermidis, or S. saccharolyticus.

### Selectivity

The present inventors surprisingly discovered that the novel EADs disclosed herein, e.g., CHAP2 (SEQ ID NO: 2) and M23-S1 (SEQ ID NO: 4), exhibited selective activity for S. aureus over S. epidermidis in the context of different chimeric proteins with different CBDs. As known in the art, the vast majority of CWHs having lytic activity towards S. aureus also have similar lytic activity towards S. epidermidis, meaning that while they can kill unwanted S. aureus overgrowth, they would also kill beneficial S. epidermidis populations, which is highly undesirable for a topical skin microbiome application. By contrast, in some embodiments, the chimeric CWHs of the present disclosure are able to distinguish between Staphylococcus aureus and Staphylococcus epidermis.

In some embodiments, a chimeric protein herein has Staphylococcus species-specific activity. In some embodiments, a chimeric protein herein has selectivity for one species of Staphylococcus over another species of Staphylococcus. Selectivity in this context refers to a chimeric protein of the disclosure that has higher activity against one species of Staphylococcus than another species of Staphylococcus. In some embodiments, a chimeric protein herein is selective for one group of Staphylococcus species over another group of Staphylococcus species. For example, in some embodiments, a chimeric protein of the disclosure is selective for the phylogenetic grouping that includes Staphylococcus aureus over the phylogenetic grouping that includes Staphylococcus epidermidis. In some embodiments, a chimeric protein of the disclosure is selective for CoPS species (e.g., Staphylococcus aureus) over CONS species (e.g., Staphylococcus epidermidis).

In some embodiments, a chimeric protein herein is selective for S. aureus over another species of Staphylococcus. In some embodiments, a chimeric protein herein is selective for S. aureus over a CONS species. In some embodiments, a chimeric protein of the disclosure is selective for S. aureus over S. epidermidis. In some embodiments, a chimeric protein of the disclosure is selective for S. aureus over S. hominis.

The degree of selectivity exhibited by a given chimeric protein against one species in comparison to another species, e.g. species A and species B, is generally defined based on a comparison of that chimeric protein's activity against species A versus its activity against species B. When activity is defined by minimum inhibitory concentration (MIC), e.g., via an MIC assay, the selectivity toward species A over species B is calculated by the inverse ratio of the MIC of the chimeric protein against each species, i.e.:

$$Fold selectivity = \frac{{MIC}_{s p e cies B}}{{MIC}_{s p e cies A}} .$$

In some embodiments, a chimeric protein of the disclosure has at least 2, 3, 4, 5, 6, 7, 8, 9, or 10-fold selectivity. In some embodiments, a chimeric protein of the disclosure has at least 5, 10, 15, 20, 25, 30, 35, 40, 45, 50, 55, 60, 65, 70, 75, 80, 85, 90, 95, or 100-fold selectivity. In some embodiments, a chimeric protein of the disclosure has virtually no activity (or no detectable activity) against one species, while having measurable activity against another species.

In some embodiments, a composition of the disclosure has broad range anti-Staphylococcus activity. In some embodiments, the composition has high activity against multiple species of Staphylococcus. In some embodiments, the composition has high activity against a group of related Staphylococcus species. In some embodiments, the composition has high activity against a diverse group of Staphylococcus species. In some embodiments, the composition is suitable for use as a broad-range therapeutic.

In some embodiments, the selectivity of a chimeric CWH of the disclosure is significantly higher than a control native protein comprising an EAD or a CBD comprised by the chimeric CWH. In some embodiments, the selectivity of the chimeric CWH is at least 2, 3, 4, 5, 6, 7, 8, 9, or 10-fold higher than for a native protein. In some embodiments, the selectivity of the chimeric CWH is at least 10, 20, 30, 40, 50, 60, 70, 80, 90 or 100-fold higher than for a native protein.

### Thermostability

In some embodiments, a composition of the disclosure exhibits thermostability at temperatures up to 37, 38, 39, 40, 41, 42, 43, 44, 45, 46, 47, 48, 49, 50, 51, 52, 53, 54, 55, 56, 57, 58, 59, or 60° C., including all values and ranges therebetween. In some embodiments, a composition of the disclosure exhibits thermostability at temperatures of at least 37, 38, 39, 40, 41, 42, 43, 44, 45, 46, 47, 48, 49, 50, 51, 52, 53, 54, 55, 56, 57, 58, 59, or 60° C., including all values and ranges therebetween.

As used herein, thermostability at a given temperature refers to the ability to maintain activity levels (e.g., a threshold activity level) at that temperature, or after the protein is exposed to that temperature. In some embodiments, thermostability at a given temperature is measured after exposure to that temperature for a period of time. In some embodiments, thermostability is determined based on experiments testing activity at a temperature or after exposure to a given temperature for a period of time (e.g., showing measurable target bacterial density reductions at that temperature or after exposure to that temperature). Thus, in some embodiments, an EAD, CBD, or chimeric protein is considered thermostable at a temperature if it still exhibits measurable activity at that temperature or after exposure to that temperature. In some embodiments, an EAD, CBD, or recombinant chimeric protein is considered thermostable at a critical temperature, if it still exhibits measurable activity at its intended use temperature after being exposed to that critical temperature (e.g., activity tested after the protein is exposed to the critical temperature for 30 mins). In some embodiments, thermostability after exposure to a given temperature is determined based on assays conducted at room temperature. In some embodiments, thermostability is determined after exposure to a given temperature based on an assay that measures activity. In some embodiments, the assay is a turbidity reduction assay.

In the context of comparing two proteins (e.g., two EADs, two CBDs, or two chimeric CWHs), one protein may be considered more thermostable at a given temperature than the other if it exhibits higher absolute activity at that temperature or after exposure to that temperature (e.g., if it results in greater microbial density reductions in the measured time). In other embodiments, one protein may be considered more thermostable than the other at a given temperature if it exhibits higher relative activity at that temperature or after exposure to that temperature (e.g., if the protein exhibits a lesser reduction in activity after exposure to a given temperature compared to the relative reduction of activity of a second protein after exposure to that same temperature).

In some embodiments, thermostability at a given temperature is determined based on ability to maintain activity after exposure to that temperature for 10, 20, 30, 40, 50, or 60 minutes, including all values and ranges therebetween. In some embodiments, thermostability at a given temperature is determined after exposure to that temperature for 1, 2, 3, 4, 5, 6, 7, 8, 9, 10, 11, 12, 13, 14, 15, 16, 17, 18, 19, 20, 21, 22, 23, or 24 hours, including all values and ranges therebetween. In some embodiments, thermostability at a given temperature is determined after exposure to that temperature for 1, 2, 3, 4, 5, 6, or 7 days, including all values and ranges therebetween. In some embodiments, thermostability at a given temperature is determined after exposure to that temperature for 1, 2, 3, or 4 weeks, including all values and ranges therebetween. In some embodiments, thermostability at a given temperature is determined after exposure to that temperature for 1, 2, 3, 4, 5, or 6 months, including all values and ranges therebetween.

In some embodiments, thermostability is measured based on testing activity after maintaining a composition at a given temperature for a period of time, e.g., weeks or months. In some embodiments, thermostability is determined based on activity retained after 2 months at a given temperature.

In some embodiments, a composition herein is thermostable at 45° C. for at least four weeks. In some embodiments, a composition herein is thermostable at 45° C. for at least two months. In some embodiments, a composition herein is thermostable at 50° C. for at least two months.

In some embodiments, a composition herein is considered thermostable or shelf stable if it retains at least 10, 20, 30, 40, 50, 60, 70, 80, or 90% of its original activity, including all values and ranges therebetween, at room temperature after exposure to a temperature of 45° C. for four weeks.

### pH Range

In some embodiments, a composition herein is stable at a range of pH values. A composition, e.g., an EAD or a chimeric protein, is considered stable at a given pH level if it exhibits activity at that pH level. In some embodiments, pH stability at different pH values is determined based on activity assays conducted at different pH values. E.g., in some embodiments, pH stability is determined by incubating a composition of the disclosure (e.g., a chimeric protein) with target bacterial cells at different pH values (e.g., in a turbidity reduction assay). The results of the assay provide activity levels for the composition at different pH values, including a maximum activity level. In some embodiments, a composition is stable at a pH level if it has the same activity at that pH as its maximum activity level. In some embodiments, a composition is stable at a pH level if it exhibits at least 50, 60, 70, 80, 90, or 95% of its maximum activity at that pH.

In some embodiments, pH stability is determined based on an activity assay. In some embodiments, the assay is a turbidity reduction assay.

In some embodiments, a composition herein is stable at a pH of 3, 4, 5, 6, 7, 8, 9, or 10, or within any ranges therebetween. In some embodiments, a composition herein is stable in the pH range of 6-8. In some embodiments, a composition herein is stable in the pH range of 5-8. In some embodiments, a composition herein is stable at pH values most relevant for topical skin applications. E.g., in some embodiments, a composition herein is stable at a pH of 4, 5, or 6.

### Synergy

In some embodiments, the present disclosure provides a combination composition comprising at least two compositions, e.g., chimeric proteins, of the disclosure. In some embodiments, at least two compositions of the disclosure are administered together. In some embodiments, two compositions of the disclosure are administered one after the other or simultaneously. In some embodiments, a combination composition of the disclosure exhibits synergistic results compared to the constituent compositions individually.

In some embodiments, a chimeric protein of the disclosure exhibits a synergistic effect from the combination of a CBD and an EAD comprised therein. In some embodiments, this synergy is measured in comparison to a control protein. In some embodiments, the control protein is the original protein from which the CBD is derived. In some embodiments, the control protein is the original protein from which the EAD is derived. In some embodiments, the control protein is a control chimera comprising the CBD and comprising a control EAD. In some embodiments, the control protein is a control chimera comprising the EAD and comprising a control CBD. In some embodiments, the control CBD is the Twort CBD. In some embodiments, the control EAD is the Twort EAD. The discussion of synergy provided herein applies to combination compositions of the disclosure and to chimeras comprising domains that interact synergistically.

As used herein, the term “synergistic” as it refers to a composition of the disclosure, refers to a composition that exhibits an effect (y) that is in excess of the predicted effect of the composition as calculated by a reference model. In some embodiments, “synergistic” refers to an effect that is greater than a simple additive effect. In some embodiments, a synergistic combination is one for which the MIC of the combination is lower than the MIC for its constituent components. In some embodiments, a synergistic combination is one for which the MIC of the combination is lower than the MIC for its constituent components, as calculated by percent composition. For example, in some embodiments, a synergistic combination of a CBD and an EAD is one in which the MIC of the CBD-EAD chimera is lower than the MIC for a control protein.

In some embodiments, the presence of synergy for a combination composition is determined based on the Fractional Inhibitory Concentration (FIC) index value. To quantify the potency of a combination of agents in comparison to the individual activities of each agent, the Fractional Inhibitory Concentration (FIC) index value for each combination of proteins is calculated using the following equation:

$$\frac{A}{{MIC}_{A}} + \frac{B}{{MIC}_{B}} = {FIC}_{A} + {FIC}_{B} = FIC Index ,$$

where A and B are the concentration of each protein in a given well and MIC_{A} and MIC_{B} are the MIC's of each protein individually; FIC_{A} and FIC_{B} are the fractional inhibitory concentrations for protein A and B, respectively, and their sum gives the overall FIC index for that well. The criteria for a determination of synergistic activity are: i) no detectable growth of a target bacterium when exposed to the combination (e.g., the combination is at a concentration equal to or greater than its MIC), and ii) an FIC index less than or equal to 0.5.

In some embodiments, a synergistic effect is calculated using the Synergyfinder web application. The following reference describes the Synergyfinder web application in detail and is incorporated by reference herein in its entirety: Ianevski, A.; He, L.; Aittokallio, T.; Tang, J. SynergyFinder: A Web Application for Analyzing Drug Combination Dose-Response Matrix Data Bioinformatics 2017, 33 (15), 2413-2415.

In some embodiments, the reference model is a “simple deduction model.” The simple deduction model determines that a composition exhibits a synergistic effect if the observed effect is greater than the effect predicted from the sum of the effects of the individual components. The synergistic effect according to the deduction model may be calculated using the following equation: y=Observed effect of composition−(sum of expected effect of individual active ingredient components). If y is greater than zero, the composition exhibits a synergistic effect. The synergy percentage adjusted model may also be calculated. The equation for the synergy percentage adjusted is: observed effect of the composition−additive inhibition value. The additive inhibition value for a composition containing two components (e.g., A and B) may be calculated according to the following equation: (Expected effect of component A)+((1−expected effect of component A)×expected effect of component B)/100.

In some embodiments, the reference model is “Highest Single Agent” (HSA). The HSA model states that the expected combination effect equals to the higher effect of individual drugs: y_{HSA}=max (y₁,y₂). The following reference describes this model in detail and is incorporated by reference herein in its entirety: Berenbaum, M. C. (1989). What is synergy? Pharmacol. Rev., 41 (2): 93-141.

In some embodiments, the reference model is the Loewe additivity model. This model defines the expected effect y_{LOEWE} as if a drug was combined with itself. the Loewe additivity model considers the dose-response curves of individual drugs. The expected effect y_{LOEWE} must satisfy:

$$\frac{{x}_{1}}{{x}_{L O E W E}^{1}} + \frac{{x}_{2}}{{x}_{L O E W E}^{2}} = 1 ,$$

where x₁ and x₂ and

$${x}_{L O E W E}^{1} and {x}_{L O E W E}^{2}$$

are the doses of component 1 1 and component 2 alone that produce y_{LOEWE}. Using 4-parameter log-logistic (4PL) curves to describe dose-response curves the following parametric form of previous equation is derived:

$$\frac{{x}_{1}}{{{m}_{1} ( \frac{{y}_{L O E W E} - {E}_{min}^{1}}{{E}_{max}^{1} - {y}_{L O E W E}} )}^{\frac{1}{{λ}^{1}}}} + \frac{{x}_{2}}{{{m}_{2} ( \frac{{y}_{L O E W E} - {E}_{min}^{2}}{{E}_{max}^{2} - {y}_{L O E W E}} )}^{\frac{1}{{λ}^{2}}}} ,$$

where Eₘᵢₙ, Eₘₐₓϵ[0,1] are minimal and maximal effects of each component, m_{1,2} are the doses of components that produce the midpoint effect of Eₘᵢₙ+Eₘₐₓ, also known as relative EC₅₀ or IC₅₀, and λ_{1,2} (λ>0) are the shape parameters for indicating the sigmoidicity or slope of dose-response curves. A numerical nonlinear solver can be then used to determine y_{LOEWE} for x₁ and x₂. The Loewe additivity model is described in detail in the following reference, which is incorporated by reference herein in its entirety: Loewe, S. (1953). The problem of synergism and antagonism of combined drugs. Arzneimit-telforschung, 3 (6): 285-290.

In some embodiments, the reference model is the Bliss model. The Bliss model is described in detail in the following reference, which is incorporated by reference herein in its entirety: Bliss, C. I. (1939). The toxicity of poisons applied jointly 1. Annals of Applied Biology, 26 (3): 585-615. Bliss assumes a stochastic process in which two components exert their effects independently, and the expected combination effect can be calculated based on the probability of independent events as: y_{BLISS}=y₁+y₂−y₁×y₂.

In some embodiments, the reference model is the Zero Interaction Potency (ZIP) model. The ZIP model is described in detail in the following reference, which is incorporated by reference herein in its entirety: Yadav, B., Wennerberg, K., Aittokallio, T., and Tang, J. (2015). Searching for Drug Synergy in Complex Dose-Response Landscapes Using an Interaction Potency Model. Comput Struct Biotechnol J, 13:504-513. ZIP calculates the expected effect of two components under the assumption that they do not potentiate each other:

$${y}_{ZIP} = \frac{{( \frac{{x}_{1}}{{m}_{1}} )}^{{λ}_{1}}}{{( 1 + \frac{{x}_{1}}{{m}_{1}} )}^{{λ}_{1}}} + \frac{{( \frac{{x}_{2}}{{m}_{2}} )}^{{λ}_{2}}}{{( 1 + \frac{{x}_{2}}{{m}_{2}} )}^{{λ}_{2}}} - \frac{{( \frac{{x}_{1}}{{m}_{1}} )}^{{λ}_{1}}}{{( 1 + \frac{{x}_{1}}{{m}_{1}} )}^{{λ}_{1}}} \cdot \frac{{( \frac{{x}_{2}}{{m}_{2}} )}^{{λ}_{2}}}{{( 1 + \frac{{x}_{2}}{{m}_{2}} )}^{{λ}_{2}}}$$

### Methods of Treating Staphylococcus Conditions

The present disclosure provides methods of treating conditions associated with Staphylococcus comprising administering a composition of the disclosure.

A composition as disclosed herein may be used to treat subjects affected by a condition associated with a Staphylococcus species as defined herein. In some embodiments, the subject is an animal. In some embodiments, the animal is a mammal. In some embodiments, the subject is a human.

### Conditions

In some embodiments, a composition of the disclosure is used in the treatment of a condition associated with Staphylococcus. In some embodiments, the condition is a Staphylococcus infection.

In some embodiments, the condition is associated with the skin. In some embodiments, the condition is a skin infection. In some embodiments, the condition is impetigo, cellulitis, folliculitis, atopic dermatitis, acute radiation dermatitis, acne, or an abscess. In some embodiments, the condition is atopic dermatitis. In some embodiments, the condition is acute radiation dermatitis. In some embodiments, the condition is dry, itchy, and/or red skin. In some embodiments, the condition is dry skin. In some embodiments, the condition is itchy skin. In some embodiments, the condition is red skin.

In some embodiments, the condition is a wound infection, pneumonia, food poisoning, toxic shock syndrome, a bloodstream infection, pneumonia, a urinary tract infection, a bone or joint infection (e.g., osteomyelitis, septic arthritis), endocarditis, meningitis, septicemia, an ear infection (e.g., otitis externa), an eye infection (e.g., conjunctivitis, keratitis), a sinus infection, gastroenteritis, mastitis, peritonitis, a prosthetic joint infection, a sternal wound infection, a catheter-related infection, or tonsillitis. In some embodiments, the condition is an infection of the skin. In some embodiments, the condition is an infection of the soft tissue. In some embodiments, the condition is an opportunistic infection. In some embodiments, the condition is a wound infection. In some embodiments, the condition is a chronic wound.

In some embodiments, the condition is the presence of Staphylococcus species within an environment, e.g., on surfaces within a hospital. In some embodiments, a composition herein is used as a disinfectant to reduce the concentration or presence of Staphylococcus species in an environment.

### Dosages

In some embodiments, a composition of the disclosure comprises 0.1-100 μg/mL of a recombinant protein, e.g., a chimeric protein, disclosed herein. In some embodiments, the composition comprises 0.5-50 μg/mL of a protein of the disclosure. In some embodiments, the composition comprises 1-25 μg/mL of a protein of the disclosure. In some embodiments, the composition comprises 2-15 μg/mL of a protein of the disclosure.

In some embodiments, a composition of the disclosure comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL of a protein of the disclosure.

### Administration

For the purposes of administration, the present compositions may be formulated in a variety of forms. The term “dosage form” denotes any form of the formulation that contains an amount of an EAD or a chimeric protein of the disclosure sufficient to achieve at least a partial therapeutic effect with a single or repeat administration. In some embodiments, the dosage form is a topical dosage form. In some embodiments, the dosage form is a lotion, an oil, a gel, a salve, or a body balm. In some embodiments, the dosage form is a lotion.

Compositions can be formulated in forms including but not limited to liquid, gel, semi-solid, and solid. Compositions disclosed herein can further be processed into forms including but not limited to solids, liquids, suspensions, gels, lotions, balms, and other forms discussed in this disclosure.

In some embodiments, an effective amount of a composition is administered to a subject. The term “effective amount” or “therapeutically effective amount” refers to that amount of a composition described herein that is sufficient to effect the intended application including but not limited to a decrease in a Staphylococcus population. The therapeutically effective amount may vary depending upon the subject and condition being treated, e.g., the weight and age of the subject, the severity of the disease condition, the manner of administration and the like, which can readily be determined by one of ordinary skill in the art. The term also applies to a dose that will induce a particular response in a target location, e.g., a reduction in inflammation, pain, acne, fever, etc. The specific dose will vary depending on the particular formulation of the composition, the dosing regimen to be followed, whether it is administered in combination with other compounds, timing of administration, the tissue to which it is administered, route of administration and the physical delivery system in which it is carried.

In some embodiments, a composition as disclosed herein is said to be active, functional or therapeutically active or able to treat, prevent and/or delay a condition associated with Staphylococcus when it reduces or ameliorates one or more symptoms associated with that condition. In some embodiments, a composition is considered therapeutically active when it decreases a symptom by at least 5, 10, 15, 20, 25, 30, 35, 40, 45, 50, 55, 60, 65, 70, 75, 80, 85, 90, 95, 96, 97, 98, 99, or 100% after treatment compared to the severity of that symptom before treatment. In some embodiments, the symptom is pain, fever, swelling, redness, dry skin, lesion number, lesion size, rash, warmness, drainage, discharge, cough, shortness of breath, rapid heart rate, low or high blood pressure, chills, nausea, vomiting, diarrhea, stomach cramps, chest pain, or organ failure.

In some embodiments, a composition herein is therapeutically active when it decreases the amount of a target Staphylococcus species present in a subject or in an in vitro system and preferably means that 99%, 90%, 80%, 70%, 60%, 50%, 40%, 30%, 20%, 10%, 5% or less of the initial amount of a Staphylococcus species, is still detectable after treatment. In some embodiments, no Staphylococcus species is detectable after treatment. Herein, the expression “amount of Staphylococcus species” refers to living Staphylococcus species. In some embodiments, Staphylococcus species are detected using sequencing techniques, such as 16S sequencing or shotgun sequencing, to quantify the amount of different Staphylococcus species present in a sample, as well as evaluating species present in the overall microbiome in question. Staphylococcus species may also be detected using standard techniques known by the artisan such as immunohistochemical techniques using Staphylococcus specific antibodies, tube coagulase tests that detect staphylocoagulase or “free coagulase”, detection of surface proteins such as clumping factor (slide coagulase test) and/or protein A (commercial latex tests). Living Staphylococcus species may be detected using standard techniques known by the artisan such as microbiological bacterial culture techniques and/or real-time quantitative reverse transcription polymerase chain reaction to assay for bacterial mRNA. In some embodiments, said decrease is assessed in a tissue or in a cell of an individual or a patient by comparison to the amount present in said individual or patient before treatment with a composition disclosed herein. In some embodiments, the comparison is made with a tissue or cell of said individual or patient which has not yet been treated with the composition as disclosed herein in case the treatment is local.

In some embodiments, application of a composition herein improves the health, appearance, quality, texture, or feel of the skin. In some embodiments, application of a composition herein reduces skin itchiness, redness, dryness, flaking, roughness, or pain.

A composition as disclosed herein may be administered to a subject in need thereof or to a cell, tissue or organ or said patient for at least one day, one week, one month, six months, one year or more. In some embodiments, a composition herein is applied for a period of 1-30 days. In some embodiments, a composition herein is applied for a period of 1-4 weeks. In some embodiments, a composition herein is applied for a period of about 1, 2, 3, 4, 5, 6, 7, 8, 9, 10, 11, or 12 months. In some embodiments, a composition herein is applied until a condition has improved. In some embodiments, a composition herein is applied to maintain a condition.

In some embodiments, a composition herein is applied 1-10 times per day. In some embodiments, a composition herein is applied 1-50 times per week. In some embodiments, a composition herein is applied as needed. In some embodiments, a composition herein is applied once or twice daily.

Accordingly, there is provided a composition as disclosed herein, for use by a subject in need thereof. Preferably, the composition is use as a medicament in the prevention, delay or treatment of a condition in a subject, wherein the condition is associated with infection with a Staphylococcus, such as a coagulase positive- or coagulase-negative Staphylococcus.

Further provided is the composition as disclosed herein for systemic or local administration to the subject.

Local administration may e.g. be used during surgery, locally at the site of infection or site of implant. The medical use disclosed herein may be formulated as a product as disclosed herein for use as a medicament for treatment of the stated conditions but can equally be formulated as a method of treatment of the stated conditions using a product as disclosed herein, a product as disclosed herein for use in the preparation of a medicament to treat the stated conditions and use of a product as disclosed herein for the treatment of the stated conditions. Such medical uses are all envisaged by the present disclosure. The subject in need of treatment, delay and/or prevention of the listed conditions may by any animal subject, preferably a mammal, more preferably cattle, domestic animals like a dog or a cat, or a human subject.

Further provided is the in vitro use of a composition as disclosed herein or a nucleic acid construct as disclosed herein, or an expression construct as disclosed herein, or a host cell as disclosed herein, as an antimicrobial or as a disinfectant.

Further provided is the use of a composition as disclosed herein or a nucleic acid construct as disclosed herein, or an expression construct as disclosed herein, or a host cell as disclosed herein, or a composition as disclosed herein, for detecting a Staphylococcus, such as Staphylococcus aureus and Staphylococcus epidermidis, in an ex vivo diagnostic application.

### EXAMPLES

### Example 1: Identification of Novel Staphylococcus-Active Cell Wall Hydrolases and Enzymatically Active Domains (EADs) Thereof

Identification of Novel Proteins. The inventors of the present application screened proteins encoded by bacteriophage that infect Staphylococcus sp. in order to identify novel cell wall hydrolases and enzymatically active domains (EADs) with activity against Staphylococcus sp. Two novel proteins were identified in this screen: a hypothetical protein from Staphylococcus phage vB_SscM-2 (Genbank accession number ANT44936.1; SEQ ID NO: 1) and a tail length tape measure protein from Staphylococcus phage StauST398-2 (Genbank accession number YP 008058813; SEQ ID NO: 3). The proteins were not previously characterized and have distinct architecture from many other anti-Staphylococcus cell wall hydrolases of the prior art. These novel enzymes were identified in the screen because they comprise EADs that potentially catalyze peptidoglycan degradation (e.g., CHAP, M23 domains) but did not have the architecture of a canonical anti-Staphylococcus multi-domain endolysin.

Identification of Novel EADs. Bioinformatics analysis was performed on YP_008058813 and ANT44936.1 to identify conserved protein domains. Analysis was performed using CDD (Lu et al., “CDD/SPARCLE: the conserved domain database in 2020,” Nucleic Acids Res 2020; 48 (D1): 265-8) and SMART (Letunic et al., “SMART: recent updates, new developments and status in 2020,” Nucleic Acids Res 2021; 49 (D1): 458-60).

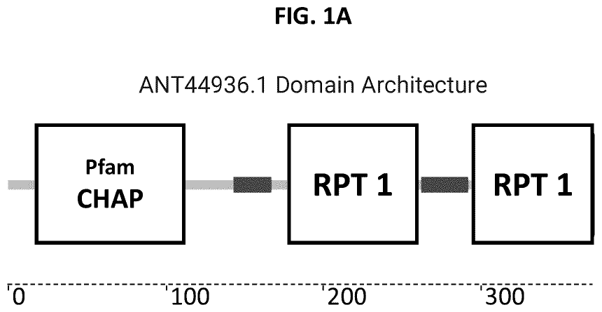

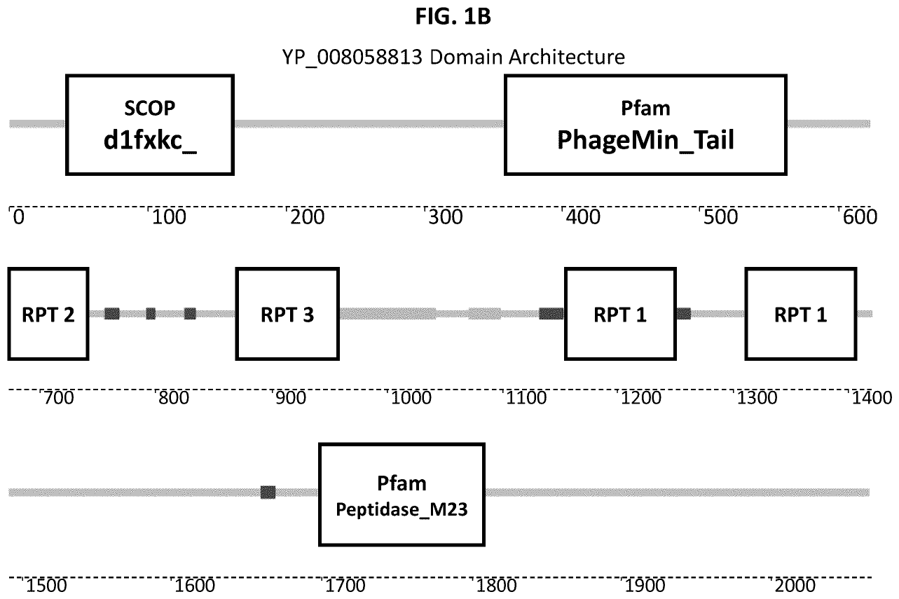

These analyses indicated that ANT44936.1 contains an N-terminal cysteine, histidine-dependent amino hydrolase/peptidase (CHAP) domain, which is a domain believed to function mainly in peptidoglycan hydrolysis; and two repeat domains of unknown function (FIG. 1A). The CHAP domain from ANT44936.1 is referred to as “CHAP2” herein (SEQ ID NO: 2).

Analysis of YP_008058813 identified a PhageMin_Tail domain, which is a well conserved core region of a family of phage tail proteins; multiple repeat domains; and an M23 peptidase domain, which has been shown in some cases to have cell wall hydrolase activity (FIG. 1B). The inventors hypothesized that YP_008058813 was a phage tail protein with an M23 enzymatic domain involved in hydrolyzing peptidoglycan cell wall to enable phage DNA injection during initial phage infection. The M23 domain from YP_008058813 is referred to as “M23-S1” herein (SEQ ID NO: 4).

### Example 2: Construction of Novel Chimeric Cell Wall Hydrolases

The inventors of the present disclosure tested the potential for the EADs from the novel proteins ANT44936.1 and YP_008058813, which are not canonical multi-domain endolysins, to function in chimeric cell wall hydrolases.

Sources for EADs and CBDs. Chimeras were constructed using the CHAP2 and M23-S1 EADs identified in Example 1 or the Twort EAD (SEQ ID NO: 143) from the Twort endolysin (SEQ ID NO: 142) as a control. The chimeras comprised cell wall binding domains (CBDs) of the following endolysins/bacteriocins: LysH5 (SEQ ID NO: 124), LysA72 (SEQ ID NO: 125), PlySs2 (SEQ ID NO: 126), ALE-1 (SEQ ID NO: 127), LysSA97 (SEQ ID NO: 128), and LysPALS1 (SEQ ID NO: 129).

Construction of chimeric cell wall hydrolases. DNA sequences of the EADs and CBDs were codon optimized and synthesized. Individual enzymatic domains including up to 50 aa upstream and downstream were synthesized with NdeI/SpeI sites. Individual cell wall binding domains including up to 25 aa upstream and downstream were synthesized with SpeI/HindIII sites. The chimeric lysins were constructed by ligating both an enzymatic domain and a cell wall binding domain into the NdeI/HindIII sites of pET24a (+). The vector also appended a C-terminal sequence encoding a 6×His protein tag used for protein purification. Chimeric enzymes consisting of all combinations of one EAD and one CBD were constructed, as shown in Table 15 below.

TABLE 15
Description and sequences of illustrative chimeric proteins.

| Chimera | SEQ | |
|---|---|---|
| Description | ID NO | Sequence |
| CHAP2 EAD + | 130 | MKTQKQVVDYIKSLEGKGWDWDNYAGWQCFDTANYHAYFAM |
| LysPALS1 CBD | | GMSLAGEGAKDIPYKNDFTGKADIYNNTPEFLAQPGDIVVWTSP |
| | | TFGGGYGHVASVISATLNTITVIEQNWLGGGISKTEVATRRSHSY |
| | | DPSMVFIRPKYAKSSTVTESKPTTVKPVTKPTSSASTPATRPVTGS |
| | | WKKNQYGTWYKPESATFVNGNQPIVTRIGSPFLNAPVGGNLPAG |
| | | ATIVYDEVCIQAGHIWIGYNAYNGNRVYCPVRTCQGVPPSHVPG |
| | | VAWGTFK |
| CHAP2 EAD + | 131 | MKTQKQVVDYIKSLEGKGWDWDNYAGWQCFDTANYHAYFAM |
| LysA72 CBD | | GMSLAGEGAKDIPYKNDFTGKADIYNNTPEFLAQPGDIVVWTSP |
| | | TFGGGYGHVASVISATLNTITVIEQNWLGGGISKTEVATRRSHSY |
| | | DPSMVFIRPKYAKSSTVTESKPTTVKPVTKPTSKNPPVPAGYTLD |
| | | KNNVPYKKEAGNYTVANVKGNNVRDGYSTNSRITGVLPNNATI |
| | | KYDGAYCINGYRWITYIANSGQRRYIATGEVDKAGNRISSFGKFS |
| | | TI |
| CHAP2 EAD + | 132 | MKTQKQVVDYIKSLEGKGWDWDNYAGWQCFDTANYHAYFAM |
| LysSA97 CBD | | GMSLAGEGAKDIPYKNDFTGKADIYNNTPEFLAQPGDIVVWTSP |
| | | TFGGGYGHVASVISATLNTITVIEQNWLGGGISKTEVATRRSHSY |
| | | DPSMVFIRPKYAKSSTVTESKPTTVKPVTKPTSPSSKPSADKITWN |
| | | WKGVFYPNPEKAIRVRKTAGLTGTVVEEDSWLYTKDDWVKFD |
| | | QVIKKDGYWWIRFKYQREGSSTNNFYCAVCRITDKEQKIKNEKY |
| | | WGTIEWA |
| CHAP2 EAD + | 133 | MKTQKQVVDYIKSLEGKGWDWDNYAGWQCFDTANYHAYFAM |
| Ale1 CBD | | GMSLAGEGAKDIPYKNDFTGKADIYNNTPEFLAQPGDIVVWTSP |
| | | TFGGGYGHVASVISATLNTITVIEQNWLGGGISKTEVATRRSHSY |
| | | DPSMVFIRPKYAKSSTVTESKPTTVKPVTKPTSMPFLKSAGYGSN |
| | | STSSSNNNGYKTNKYGTLYKSESASFTANTDIITRLTGPFRSMPQS |
| | | GVLRKGLTIKYDEVMKQDGHVWVGYNTNSGKRVYLPVRTWNE |
| | | STGELGPLWGTIK |
| CHAP2 EAD + | 134 | MKTQKQVVDYIKSLEGKGWDWDNYAGWQCFDTANYHAYFAM |
| PlySs2 CBD | | GMSLAGEGAKDIPYKNDFTGKADIYNNTPEFLAQPGDIVVWTSP |
| | | TFGGGYGHVASVISATLNTITVIEQNWLGGGISKTEVATRRSHSY |
| | | DPSMVFIRPKYAKSSTVTESKPTTVKPVTKPTSSRSYRETGTMTV |
| | | TVDALNVRRAPNTSGEIVAVYKRGESFDYDTVIIDVNGYVWVSY |
| | | IGGSGKRNYVATGATKDGKRFGNAWGTFK |
| CHAP2 EAD + | 135 | MKTQKQVVDYIKSLEGKGWDWDNYAGWQCFDTANYHAYFAM |
| LysH5 CBD | | GMSLAGEGAKDIPYKNDFTGKADIYNNTPEFLAQPGDIVVWTSP |
| | | TFGGGYGHVASVISATLNTITVIEQNWLGGGISKTEVATRRSHSY |
| | | DPSMVFIRPKYAKSSTVTESKPTTVKPVTKPTSSNDSSASSNTVKP |
| | | VASAWKRNKYGTYYMEESARFTNGNQPITVRKVGPFLSCPVGY |
| | | QFQPGGYCDYTEVMLQDGHVWVGYTWEGQRYYLPIRTWNGSA |
| | | PPNQILGDLWGEIS |
| M23-S1 EAD + | 136 | MDGSYLFEYPIWQRFGRYTGGLNFNGGRHYGIDFGMPSGTNVY |
| LysPALS1 CBD | | AVKGGIADKVWTDYGGGNSIQIKTGANEWNWYMHLSKQLARQ |
| | | GQRIKAGQLIGKSGATGNFVRGAHLHFQLMQGSHPGNDTAKDP |
| | | EKWLKSLKGSGVRSGSGTSSASTPATRPVTGSWKKNQYGTWYK |
| | | PESATFVNGNQPIVTRIGSPFLNAPVGGNLPAGATIVYDEVCIQA |
| | | GHIWIGYNAYNGNRVYCPVRTCQGVPPSHVPGVAWGTFK |
| M23-S1 EAD + | 137 | MDGSYLFEYPIWQRFGRYTGGLNFNGGRHYGIDFGMPSGTNVY |
| LysA72 CBD | | AVKGGIADKVWTDYGGGNSIQIKTGANEWNWYMHLSKQLARQ |
| | | GQRIKAGQLIGKSGATGNFVRGAHLHFQLMQGSHPGNDTAKDP |
| | | EKWLKSLKGSGVRSGSGTSKNPPVPAGYTLDKNNVPYKKEAGN |
| | | YTVANVKGNNVRDGYSTNSRITGVLPNNATIKYDGAYCINGYR |
| | | WITYIANSGQRRYIATGEVDKAGNRISSFGKFSTI |
| M23-S1 EAD + | 138 | MDGSYLFEYPIWQRFGRYTGGLNFNGGRHYGIDFGMPSGTNVY |
| LysSA97 CBD | | AVKGGIADKVWTDYGGGNSIQIKTGANEWNWYMHLSKQLARQ |
| | | GQRIKAGQLIGKSGATGNFVRGAHLHFQLMQGSHPGNDTAKDP |
| | | EKWLKSLKGSGVRSGSGTSPSSKPSADKITWNWKGVFYPNPEKA |
| | | IRVRKTAGLTGTVVEEDSWLYTKDDWVKFDQVIKKDGYWWIRF |
| | | KYQREGSSTNNFYCAVCRITDKEQKIKNEKYWGTIEWA |
| M23-S1 EAD + | 139 | MDGSYLFEYPIWQRFGRYTGGLNFNGGRHYGIDFGMPSGTNVY |
| Ale1 CBD | | AVKGGIADKVWTDYGGGNSIQIKTGANEWNWYMHLSKQLARQ |
| | | GQRIKAGQLIGKSGATGNFVRGAHLHFQLMQGSHPGNDTAKDP |
| | | EKWLKSLKGSGVRSGSGTSMPFLKSAGYGSNSTSSSNNNGYKTN |
| | | KYGTLYKSESASFTANTDIITRLTGPFRSMPQSGVLRKGLTIKYDE |
| | | VMKQDGHVWVGYNTNSGKRVYLPVRTWNESTGELGPLWGTIK |
| M23-S1 EAD + | 140 | MDGSYLFEYPIWQRFGRYTGGLNFNGGRHYGIDFGMPSGTNVY |
| PlySs2 CBD | | AVKGGIADKVWTDYGGGNSIQIKTGANEWNWYMHLSKQLARQ |
| | | GQRIKAGQLIGKSGATGNFVRGAHLHFQLMQGSHPGNDTAKDP |
| | | EKWLKSLKGSGVRSGSGTSSRSYRETGTMTVTVDALNVRRAPN |
| | | TSGEIVAVYKRGESFDYDTVIIDVNGYVWVSYIGGSGKRNYVAT |
| | | GATKDGKRFGNAWGTFK |
| M23-S1 EAD + | 141 | MDGSYLFEYPIWQRFGRYTGGLNFNGGRHYGIDFGMPSGTNVY |
| LysH5 CBD | | AVKGGIADKVWTDYGGGNSIQIKTGANEWNWYMHLSKQLARQ |
| | | GQRIKAGQLIGKSGATGNFVRGAHLHFQLMQGSHPGNDTAKDP |
| | | EKWLKSLKGSGVRSGSGTSSNDSSASSNTVKPVASAWKRNKYG |
| | | TYYMEESARFTNGNQPITVRKVGPFLSCPVGYQFQPGGYCDYTE |
| | | VMLQDGHVWVGYTWEGQRYYLPIRTWNGSAPPNQILGDLWGE |
| | | IS |

Protein production and purification. The expression vectors containing the chimeric cell wall hydrolases were chemically transformed into inducible BL21 E. coli expression vectors for downstream protein expression and purification. BL21 cells containing the appropriate expression plasmid were grown in 2×YT media overnight at 37° C. with shaking. The next morning, cells were back diluted 1:1000 in flasks containing 50 mL of ZYM-5052 autoinduction media (Fisher Scientific™ Cat No. NC1093977) and incubated with shaking for 2-3 h at 37° C. Flasks were then transferred to 22° C. and incubated with shaking overnight. Cultures were spun down, supernatant was poured off, and pellets were stored at −80° C. for at least 30 min. Each frozen pellet was resuspended in 5 mL of lysis buffer (NPI-10 (100 μM Tris pH 8, 300 mM NaCl, 10 mM imidazole) with the addition of 5 mg lysozyme and 100 units of DNASeI) and incubated at 30° C. with gentle shaking for 30 mins. Cells were then spun down until a clear lysate was obtained and a solid pellet formed. The clear lysate was transferred to a column containing Nickel-NTA Agarose Resin (Gold Bio) suspended in NPI-10. Columns were inverted several times to completely resuspend the resin and were then incubated at 4° C. for a minimum of 1 h to allow for protein binding. Once the resin was completely settled, the lysate was allowed to run off the column and the columns were washed with two column volumes of NPI-20 (100 UM Tris pH 8, 300 mM NaCl, 20 mM imidazole). Proteins were then eluted by adding 3 mL of NPI-250 (100 uM Tris pH 8, 300 mM NaCl, 250 mM imidazole) and fractions were collected. Proteins were quantified and purity checked via Bradford assay and SDS-PAGE gel and Coomassie staining. Proteins were then concentrated and buffer exchanged into protein storage buffer (50 mM Tris pH 6.8, 300 mM NaCl) using Amicon® Ultra-15 Filter Units. For long term storage, proteins were stored at −80° C. with 30% glycerol.

### Example 3: Experimental Assays to Test Anti-Staphylococcus Activity of Illustrative Chimeras

The chimeras of Example 2 were tested in various assays to determine activity against different Staphylococcus sp., as described in Examples 4-7 below, using the following materials and methods.

Bacterial strains and culture conditions. After purification of the proteins in Example 2, the chimeric enzymes were tested for lytic activity against Staphylococcus sp., as described in Examples 4-7 below. All the staphylococcal strains were grown in tryptic soy broth (TSB; BD Difco™, Franklin Lakes, NJ) at 37° C. with shaking or on TSB plates containing 2% (w/v) agar. Escherichia coli DH10B (Invitrogen™, Carlsbad, CA) was used for cloning and storage. E. coli BL21 (DE3) (EMD Biosciences, San Diego, CA) was used for protein expression. All E. coli strains were grown at 37° C. with shaking in 2×YT medium (16 g/L tryptone, 10 g/L yeast extract, 5 g/L NaCl) or on plates containing LB (10 g/L tryptone, 10 g/L NaCl, 5 g/L yeast extract) supplemented with 2% (w/v) agar. 50 μg/ml kanamycin was used for proper selection of E. coli clones.

Turbidity Reduction Assays. Lytic activity of chimeric cell wall hydrolases were assayed via turbidity reduction assay as described in the art. Briefly, Staphylococcus sp. of interest were grown overnight in TBS at 37° C. with shaking. In the morning, cells were diluted back and allowed to grow to exponential phase (OD600 ˜0.5) in TBS at 37° C. with shaking (˜2-3 hours). Approximately 1×10⁶ cells per reaction were then mixed with 2-fold dilutions of purified protein (e.g., the chimeric enzymes and/or controls at example concentrations of 12 μg/mL to 0.75 μg/mL) in a final volume of 200 μl of PBS in a flat bottom microtiter plate. The OD600 of each well was then measured every two minutes using a microplate reader. Lytic activity of the cell wall hydrolases leads to a loss of Staphylococcus cell integrity, which leads to a reduction in the OD600 reading. More rapid decreases in optical density are correlated with higher enzymatic activities. Specific activity was calculated as described in Briers et al., “A standardized approach for accurate quantification of murein hydrolase activity in high-throughput assays,” J Biochem Biophys Methods 2007; 70 (3): 531-3.

Thermostability assay. To test thermostability of proteins, an aliquot of the protein was incubated for 30 min at temperatures ranging from 37° C. to 51° C. Proteins were then immediately tested in room temperature turbidity reductions assays as described above. Activity results from heat-exposed proteins were compared amongst each other to evaluate each enzyme's ability to maintain activity over a range of temperatures, with results at 37° C. serving as a positive control for enzyme activity.

Minimum Inhibitory Concentration (MIC) Assays. The MIC of the proteins was determined by a conventional broth microdilution technique in TSB (CLSI. Methods for Dilution Antimicrobial Susceptibility Tests for Bacteria that Grow Aerobically, 11th ed. CLSI standard M07. Clinical and Laboratory Standards Institute; 2018.). Briefly, progressive two-fold dilutions of each protein were added to a microtiter plate, with each well containing 1×10⁶ cells of the target Staphylococcal sp. The MIC was defined as the lowest protein concentration that inhibited visible bacterial growth after 24 h incubation at 37° C.

Quantitative Killing Experiments. The antimicrobial activity of chimeric cell wall hydrolases were assayed via quantitative killing assays as described in the art. Briefly, Staphylococcus sp. of interest were grown overnight in TBS at 37° C. with shaking. In the morning, cells were diluted back and allowed to grow to exponential phase (OD600 ˜0.5) in TBS at 37° C. with shaking (˜2-3 h). Approximately 1×10⁶ cells per reaction were then mixed with the desired amount of protein in a final volume of 200 μl of TSB media and incubated at room temperature. At the appropriate time points (e.g., 0 time point and the 2 h time point), 20 μL of the reaction was removed and serial dilutions were plated on TSB agar plates and grown for ˜16 hours at 37° C. CFUs were then counted to calculate the number of viable cells.

### Example 4: Novel CHAP2 EAD Shows Inherent Selectivity for S. aureus Over S. epidermidis in Illustrative Chimeras of the Disclosure

CHAP2 EAD-comprising chimeras were tested in turbidity reduction assays and MIC assays, as described in Example 3. CHAP2 chimeras were compared to a Twort EAD+PlySs2 CBD chimera (SEQ ID NO: 144) as a control.

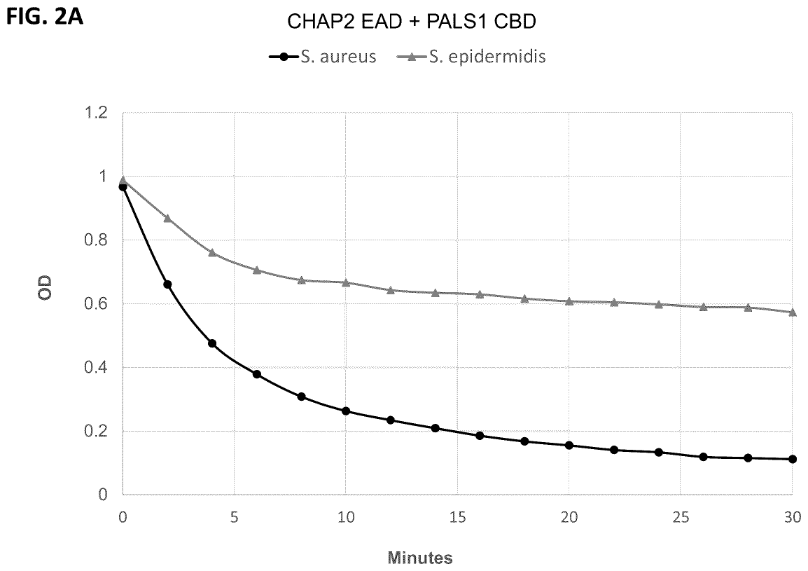

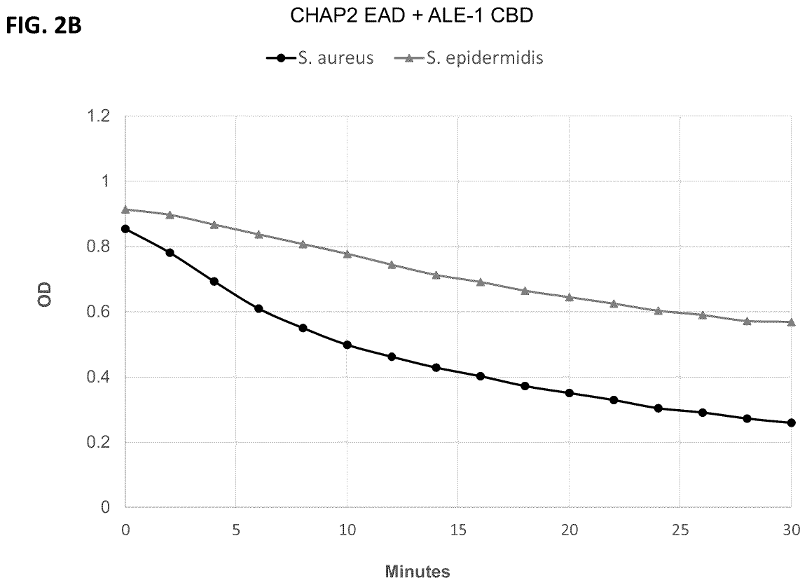

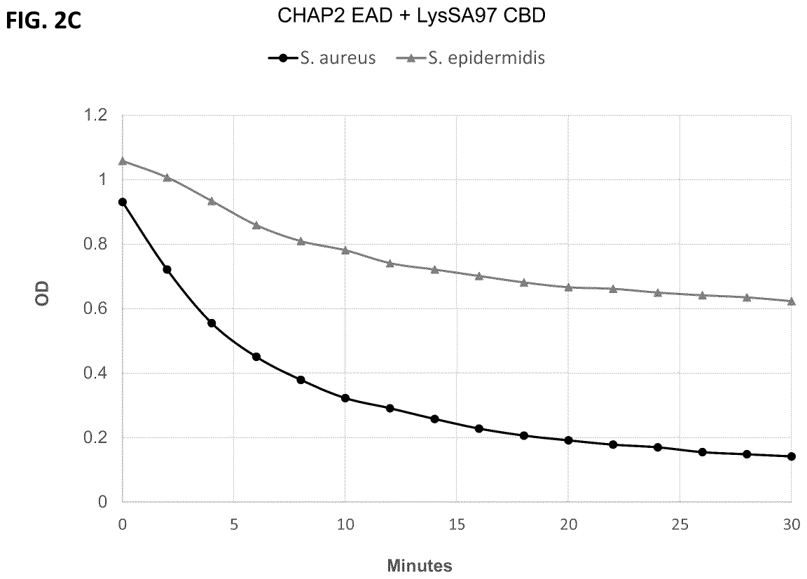

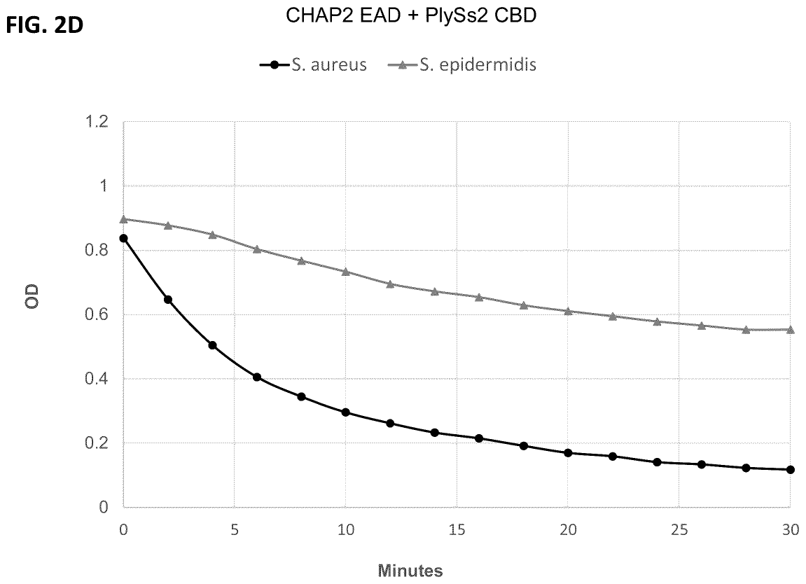

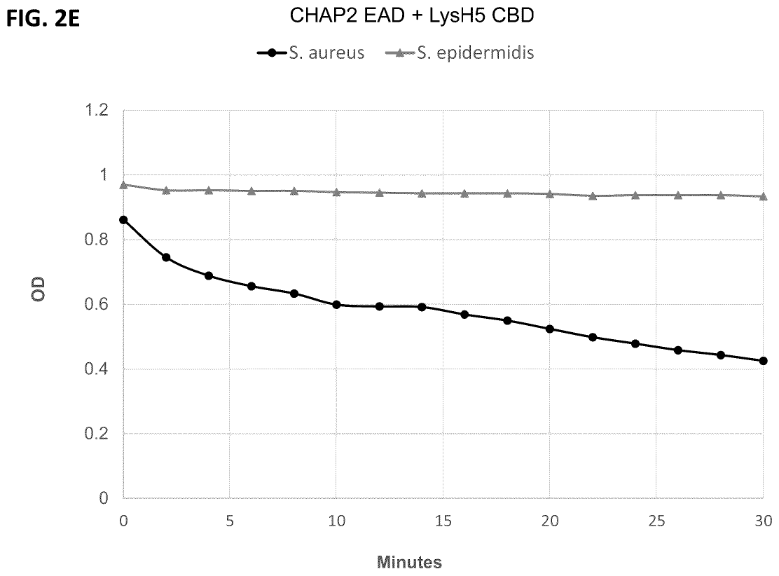

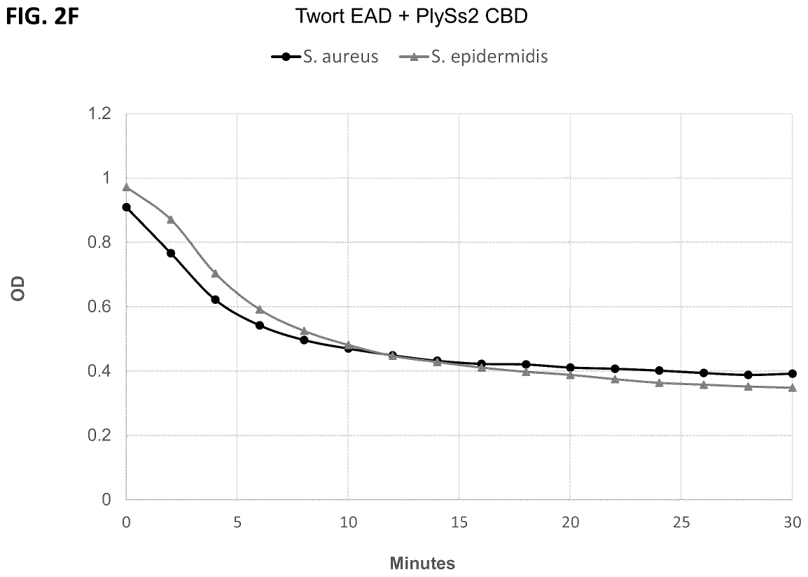

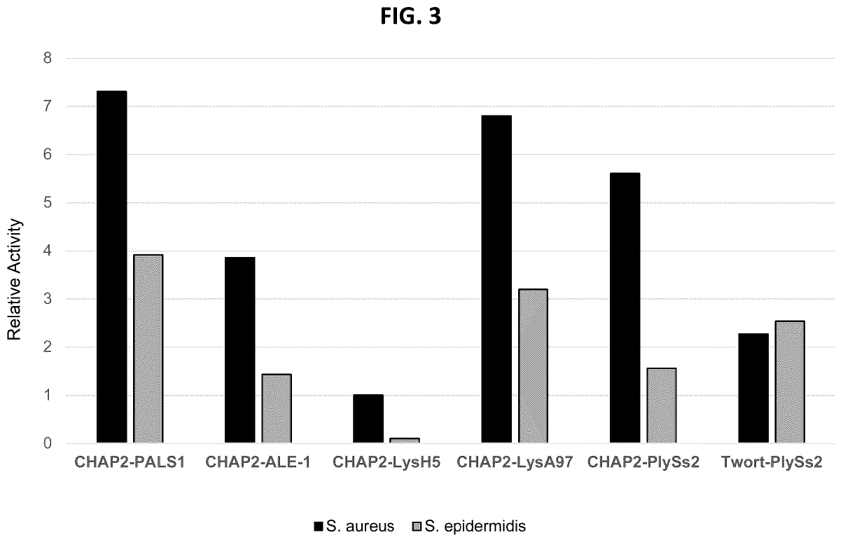

Turbidity Reduction Assay Results: In these assays, all chimeras were tested at a concentration of 12 μg/mL. All chimeric enzymes containing the CHAP2 EAD were found to have strong lytic activity towards S. aureus as demonstrated by a rapid decrease in OD600 of S. aureus in the assay (FIG. 2A-2E). These results demonstrate that the CHAP2 EAD can be broadly utilized in a variety of multi-domain chimeric cell wall hydrolases (e.g., in combination with multiple different CBDs) to impart activity against S. aureus. Surprisingly, these chimeric enzymes were significantly less active towards S. epidermidis, as demonstrated by the much slower decrease in OD of S. epidermidis. By contrast, the Twort EAD+PlySs2 CBD chimera was assayed in the turbidity reduction assay and showed equivalent activity against S. aureus and S. epidermidis. The results in FIG. 2A-2E were used to calculate relative activity by normalizing all activity (represented as change in OD per mg of protein per min) to the corresponding CHAP2 EAD+LysH5 CBD activity against S. aureus (arbitrarily setting this to 1 for ease of calculation). Based on the calculation of relative activity, each chimeric enzyme was 2-14× more active against S. aureus compared to S. epidermidis (FIG. 3). Without wishing to be bound by any particular theory, the inventors hypothesize that all chimeric enzymes containing the CHAP2 EAD demonstrated selectivity for S. aureus over S. epidermidis because the selectivity is inherent to the CHAP2 EAD. To the inventors' best knowledge, this is the first documented example of a CHAP domain that can distinguish between S. aureus and S. epidermidis.

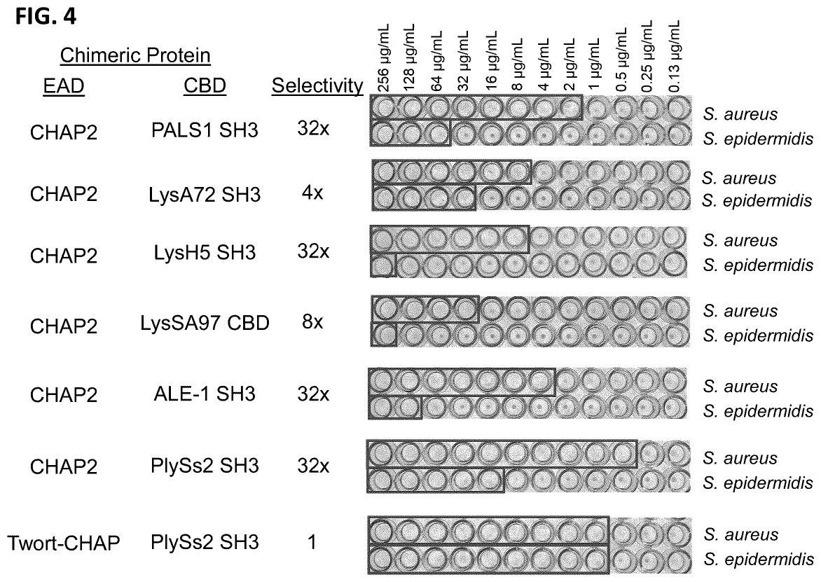

Minimum Inhibitory Concentration (MIC) Assay Results: To further evaluate the inherent S. aureus selectivity of the CHAP2 EAD, the chimeric enzymes containing the CHAP2 EAD were tested in minimum inhibitory concentration (MIC) assays against S. aureus and S. epidermidis (FIG. 4). The MIC values for these chimeric enzymes against S. aureus ranged from 0.5 μg/mL to 32 μg/mL. Consistent with the turbidity reduction assays, the MIC values against S. epidermidis were consistently 4-32× higher than those against S. aureus for all CHAP2-comprising chimeric enzymes, supporting the conclusion that the CHAP2 EAD is inherently selective for S. aureus over S. epidermidis. As a control, the Twort EAD+PlySs2 chimera displayed equal MIC values against S. aureus and S. epidermidis.

### Example 5: Illustrative Chimeras Comprising M23-S1 EAD Show High Anti-Staphylococcus Activity and Thermostability Up to 50° C.

M23-S1 EAD-comprising chimeras were tested in turbidity reduction assays and thermostability assays, as described in Example 3. M23-S1 chimeras were compared to a Twort EAD+PlySs2 CBD chimera as a control.

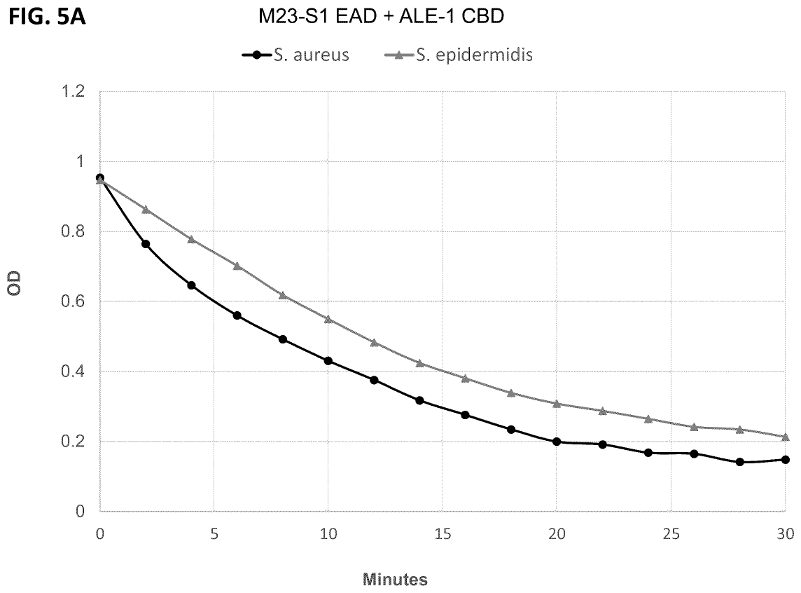

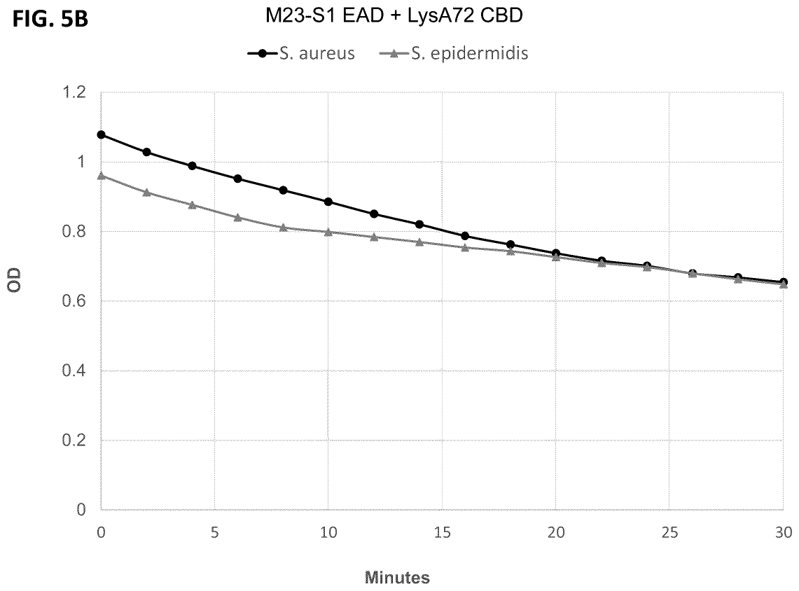

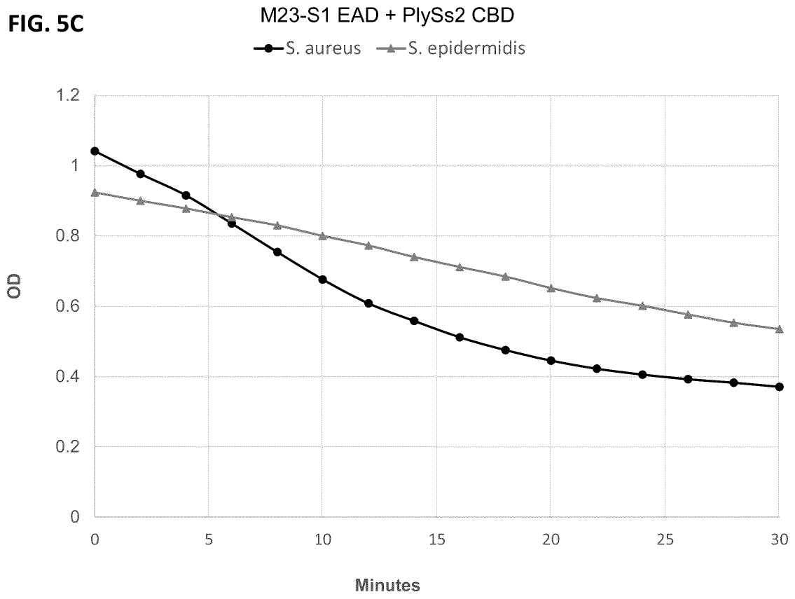

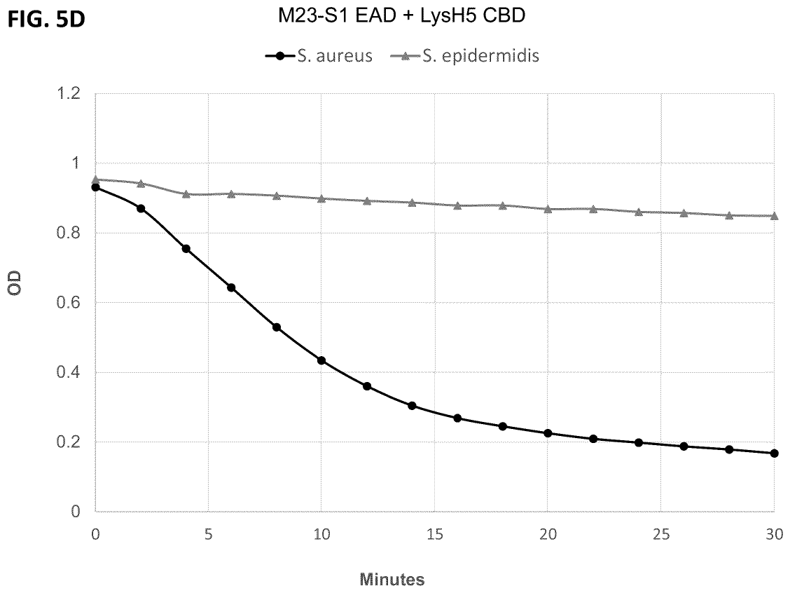

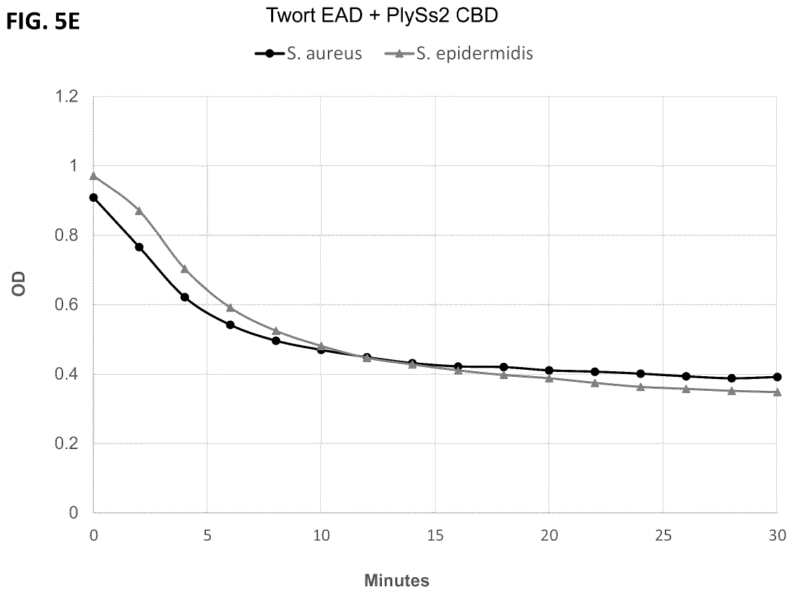

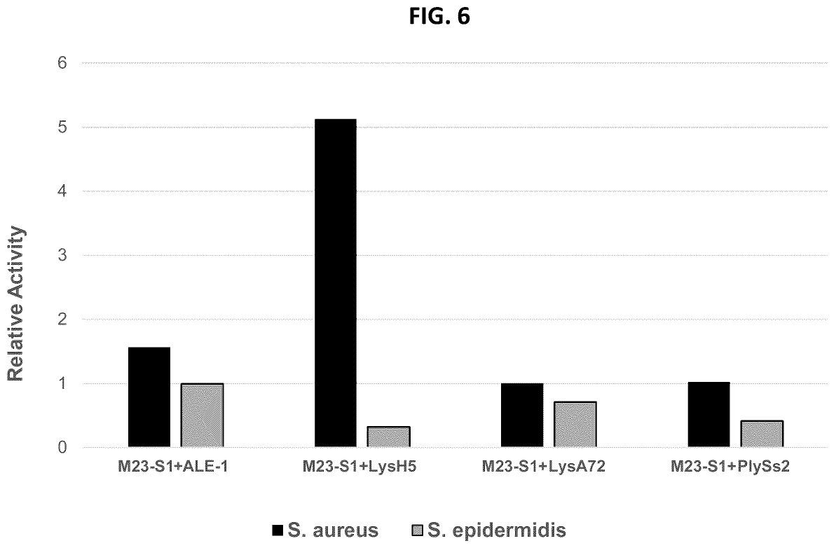

Turbidity Reduction Assay Results: All chimeric enzymes containing the M23-S1 EAD exhibited significant lytic activity against S. aureus in turbidity reduction assays, indicating that the M23-S1 EAD can be broadly utilized in a variety of multi-domain chimeric cell wall hydrolases (e.g., in combination with multiple different CBDs) to impart activity against Staphylococcus sp. (FIG. 5A-5E). These chimeric enzymes were also tested against S. epidermidis. To calculate relative activity, activities were normalized to M23-S1 EAD+PlySs2 activity against S. aureus (FIG. 6). The M23-S1 EAD+ALE-1 CBD and M23-S1 EAD+LysA72 CBD showed similar lytic activity against S. epidermidis and S. aureus. M23-S1 EAD+PlySs2 CBD showed 2.5× higher activity towards S. aureus than S. epidermidis. Most strikingly, the M23-S1 EAD+LysH5 CBD chimeric protein displayed highly selective activity with 16-fold higher specific activity towards S. aureus compared to S. epidermidis.

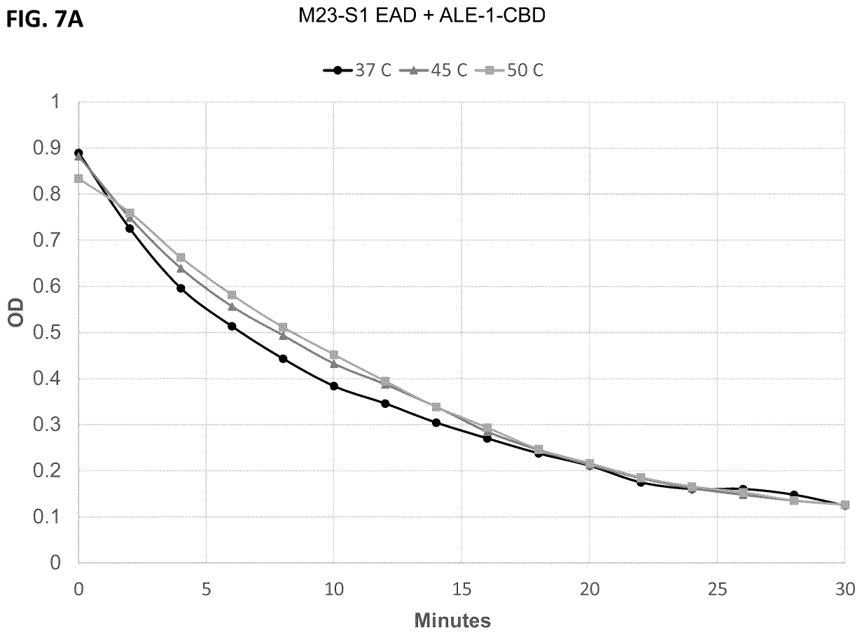

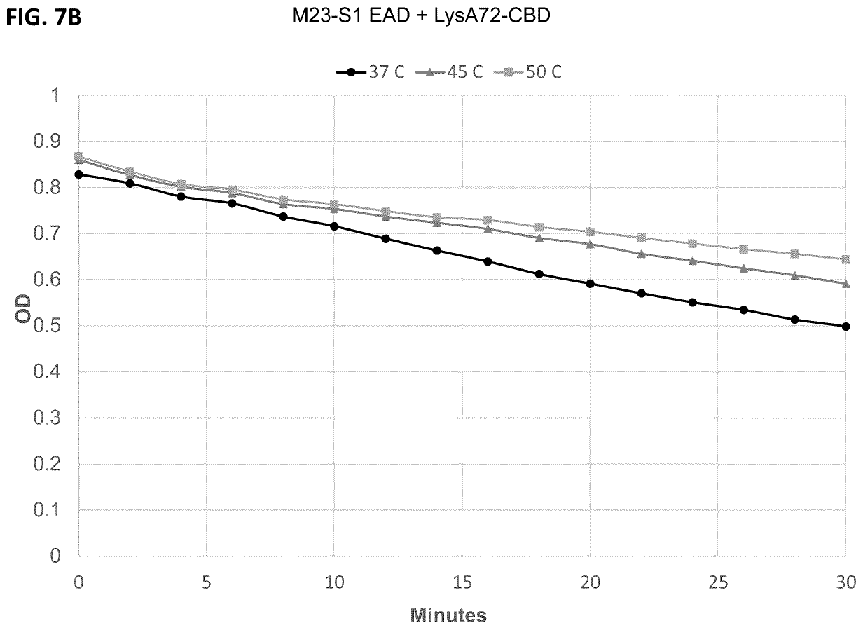

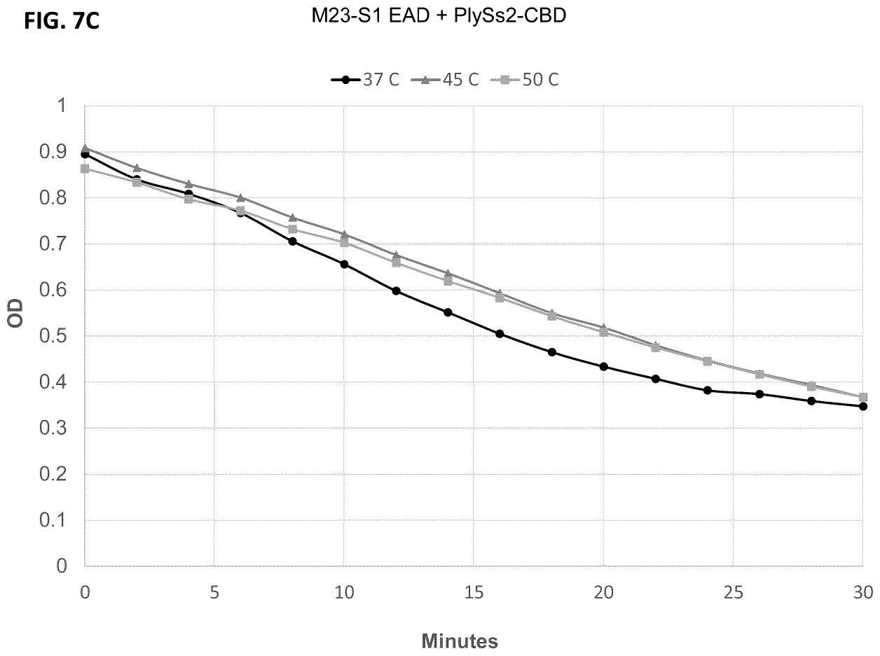

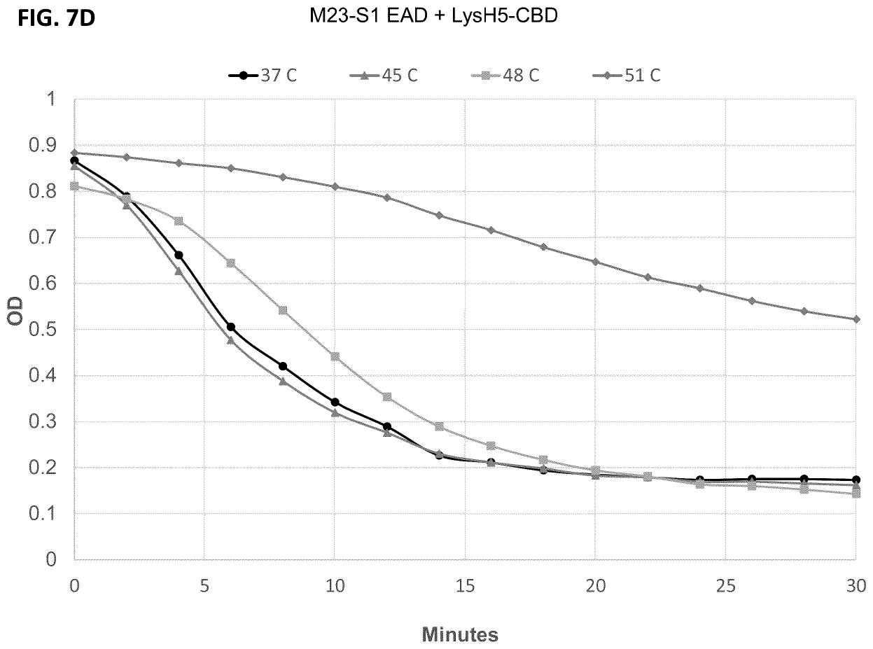

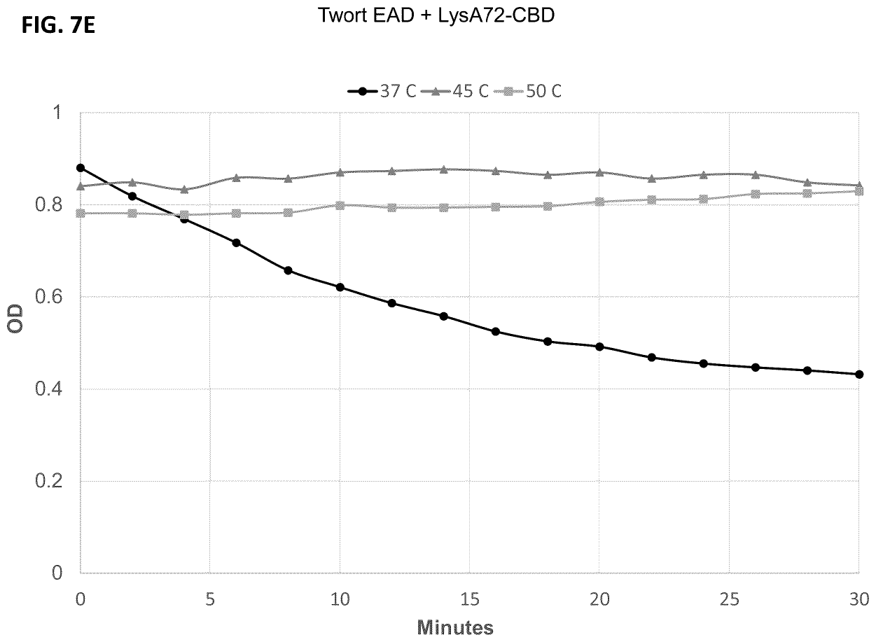

Thermostability Assay Results: Thermostability of enzymes is an important factor in potential commercial applications. To assess the thermostability of the M23-S1 chimeric enzymes, the aliquots of the enzymes were incubated for 30 min at temperatures ranging from 37-51° C. and then tested in a standard turbidity reduction assay at room temperature. Surprisingly, all M23-S1 chimeric enzymes retained activity up to 48-50° C. which is significantly higher than the majority of cell wall hydrolases that target Staphylococcus sp. (FIG. 7A-7D). For example, a control Twort-LysA72 chimeric enzyme (SEQ ID NO: 145) lost all activity at after being exposed to any temperature of 45° C. or higher (FIG. 7E).

### Example 6: An Illustrative M23-S1 EAD+LysH5 CBD Chimera Demonstrates High Activity for S. aureus and Strong Selectivity for S. aureus Over S. epidermidis

The combination of very high activity and high selectivity of the M23-S1 EAD+LysH5 CBD makes this chimera particularly well-suited for any application for which there is a need to selectively remove S. aureus from a community of microorganisms that also includes other Staphylococcus sp. (e.g S. epidermidis). The skin microbiome is one example of such a community, where S. epidermidis is a commensal organism and key component of a healthy microbiome, while S. aureus is typically pathogenic and associated with skin conditions such as atopic dermatitis.

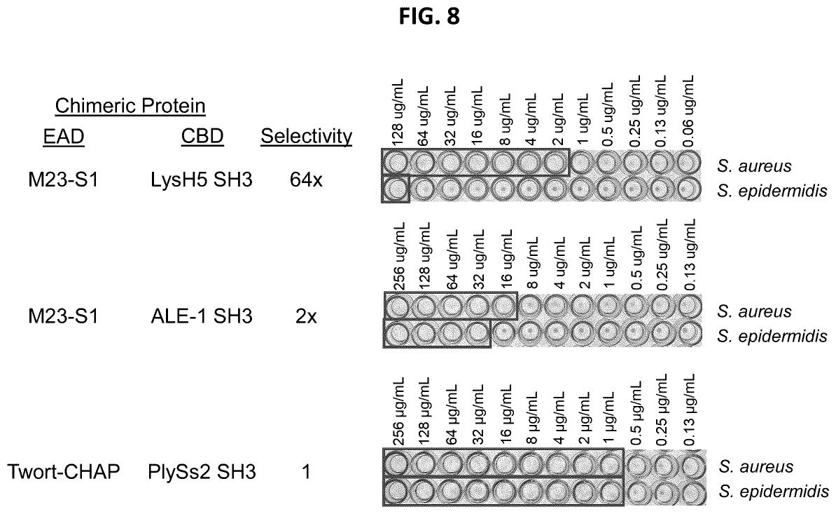

To further confirm the strong and selective activity of the M23-S1 EAD+LysH5 CBD chimeric protein, MIC assays were performed against S. aureus and S. epidermidis (FIG. 8). Consistent with the turbidity reduction assays, M23-S1 EAD+LysH5 CBD had an MIC value of 2 μg/mL against S. aureus and 128 μg/mL against S. epidermidis, representing a 64-fold difference in activity. By comparison, the M23-S1 EAD+ALE-1 CBD chimera only had 2-fold selectivity, while the Twort EAD+PlySs2 CBD chimera showed no selectivity.

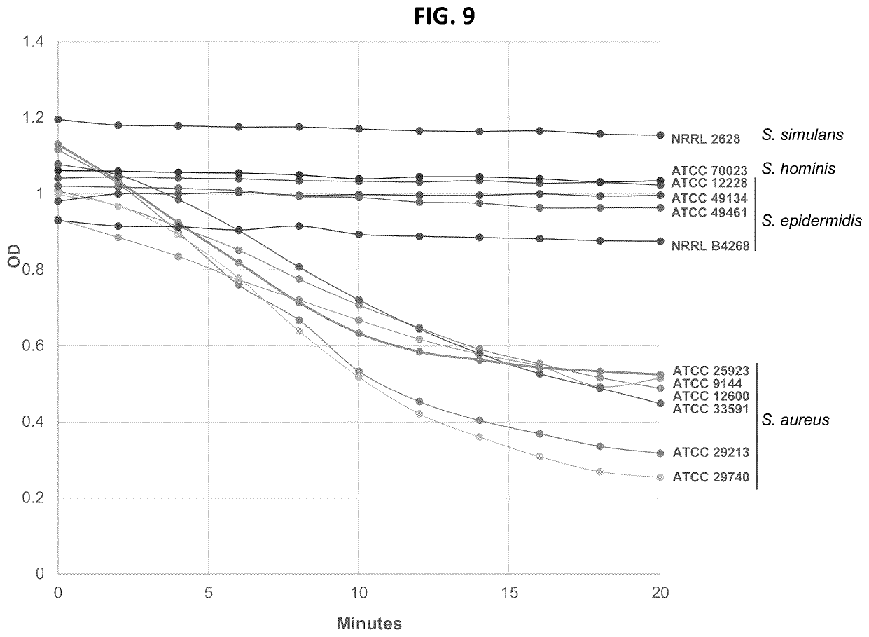

To ensure that this selectivity was consistent across a wider panel of Staphylococcus sp., M23-S1 EAD+LysH CBD was assayed in turbidity reduction assays against six S. aureus strains, four S. epidermidis strains, one S. hominis strain, and one S. simulans strain (FIG. 9). In all cases, S. aureus strains were sensitive to lysing by M23-S1 EAD+LysH5 CBD while the S. epidermidis, S. hominis, and S. simulans strains were more resistant.

### Example 7: An Illustrative M23-S1 EAD+LysH5 CBD Chimera Outperforms a Commercially Available Chimeric Endolysin in Terms of Activity and Selectivity

SA.100 is a well-known commercialized chimeric endolysin that has been shown to have selectivity for S. aureus over S. epidermidis (Staphefekt™, see www.staphefekt.com/en/). The M23-S1 EAD+LysH5 CBD chimera was compared to SA. 100 in a turbidity reduction assay and in a quantitative killing assay.

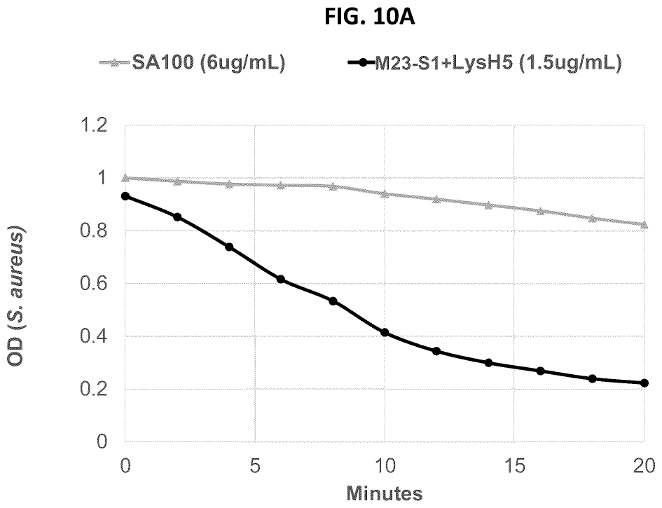

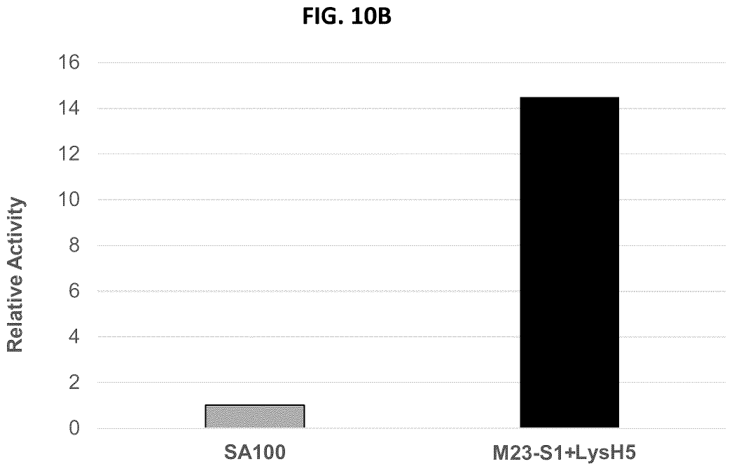

Turbidity Reduction Assay Results: The activity of M23-S1 EAD+LysH5 CBD was compared to SA.100 in a turbidity reduction assay against S. aureus. Relative activity was calculated by normalizing all activities to SA. 100 activity against S. aureus. The specific activity of M23-S1 EAD+LysH5 CBD was ˜14 fold higher than that of SA.100 (FIG. 10A-10B), indicating that M23-S1 EAD+LysH5 CBD is significantly more effective than SA. 100 at lysing S. aureus.

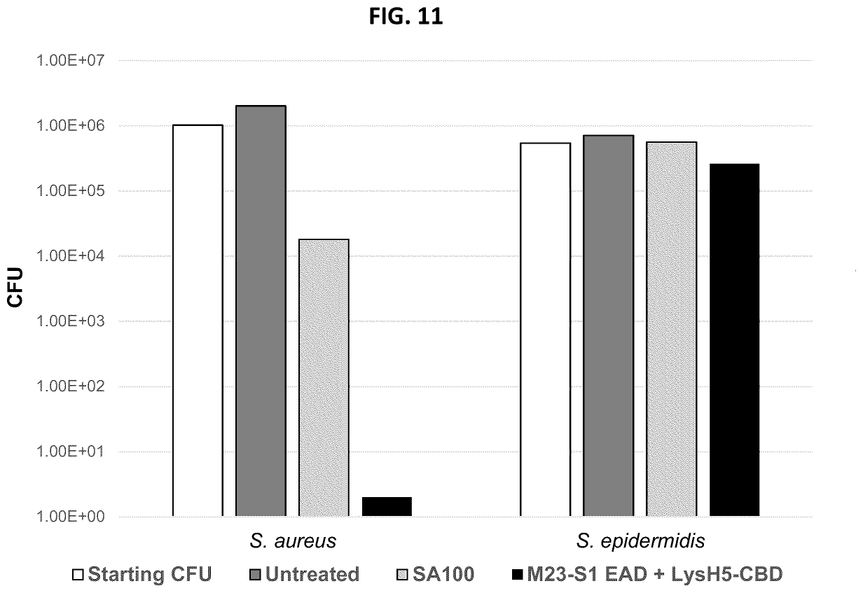

Quantitative Killing Assay Results: Quantitative killing assays were then performed to compare the effects on cell viability of S. aureus and S. epidermidis when incubated with SA.100 and M23-S1 EAD+LysH5 CBD (FIG. 11). After two hours of incubation, treatment with SA.100 resulted in a decrease of 2 orders of magnitude of viable S. aureus cells. In comparison, 2 hour incubation with M23-S1 EAD+LysH5 CBD resulted in a decrease of 5 orders of magnitude of viable S. aureus, again demonstrating that M23-S1 EAD+LysH5 CBD is significantly more potent than SA.100. While neither SA.100 nor M23-S1 EAD+LysH5 CBD had strong activity against S. epidermidis in these quantitative killing assays, M23-SIEAD+LysH5 CBD had significantly greater fold selectivity for S. aureus over S. epidermidis.

### Example 8: Staphylococcus aureus-Selective Activity of the M23-S1 EAD+LysH5 CBD Chimeric Protein in a 3D Skin Model of the Skin Microbiome

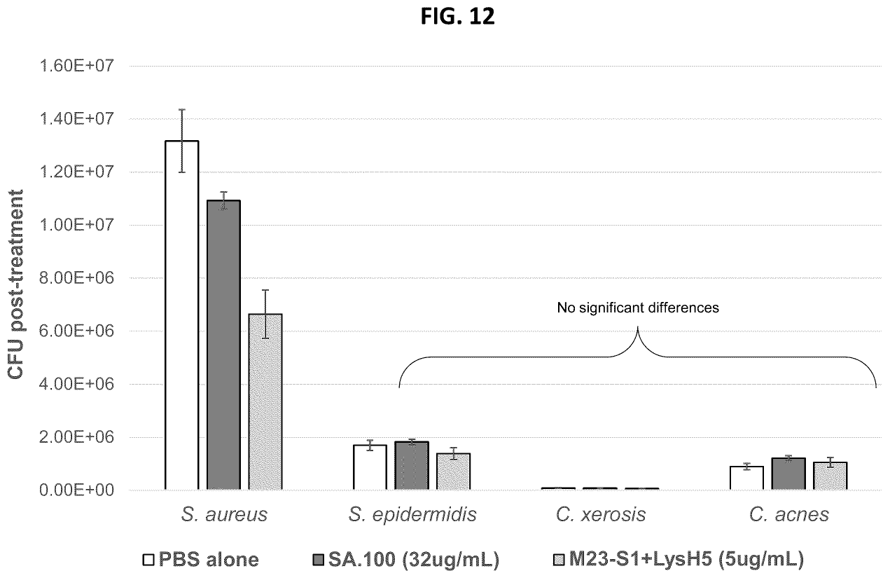

Given the strong, S. aureus-selective activity of the M23-S1 EAD+LysH5 CBD chimeric protein in MIC, turbidity reduction, and quantitative killing assays, the effects of this protein were tested on a 3D skin model of the skin microbiome to demonstrate suitability for topical applications. In this experiment, reconstituted human epidermis (RHE) was colonized overnight with a mixture of 4 skin bacteria: Staphylococcus aureus, Staphylococcus epidermidis, Corynebacterium xerosis, and Cutibacterium acnes. The RHE was then treated with solutions of M23-S1 EAD+LysH5 CBD, SA.100, or PBS alone for 4 hours. The RHE was washed gently three times and then the remaining adherent bacterial cells were recovered and quantified using quantitative PCR. Results are shown in FIG. 12. Treatment with M23-S1 EAD+LysH5 CBD resulted in a 3-fold greater decrease in adherent S. aureus cells compared to treatment with SA. 100. No significant differences were observed in the amount of S. epidermidis, C. xerosis, or C. acnes treated with either protein compared to the PBS control, showing a selective reduction in S. aureus consistent with the results of preceding examples.

### Example 9: Synergistic Anti-Staphylococcus aureus Effect from Combination of M23 EAD-Comprising Chimera and CHAP2 EAD-Comprising Chimera

Checkerboard Assay. A standard checkerboard assay was used to measure potential synergistic activity produced by a combination of chimeric CWHs. This assay measures the minimum inhibitory concentration (MIC) of the combined proteins in double serial dilution. Briefly, in a 96-well microtiter plate, columns 1-11 contained 2-fold serial dilutions of Protein A while rows A-G contained 2-fold serial dilutions of Protein B. Row H contained Protein A alone and column 12 contained Protein B alone, which served as controls for measuring the MIC's of Protein A and B alone. Approximately 1×10⁶ S. aureus cells were added to each well in a final combined volume of 200 μL of TSB. Visible growth of S. aureus was assessed after 20 hours of incubation at 37° C. To quantify the potency of the combination of antibiotics in comparison to their individual activities, the Fractional Inhibitory Concentration (FIC) index value for each combination of proteins was calculated using the following equation:

$$\frac{A}{{MIC}_{A}} + \frac{B}{{MIC}_{B}} = {FIC}_{A} + {FIC}_{B} = FIC Index ,$$

where A and B are the concentration of each protein in a given well and MIC_{A} and MIC_{B} are the MIC's of each protein individually; FIC_{A} and FIC_{B} are the fractional inhibitory concentrations for protein A and B, respectively, and their sum gives the overall FIC index for that well. The criteria for a determination of synergistic activity were: i) no detectable growth of S. aureus within the well, and ii) an FIC index less than or equal to 0.5.

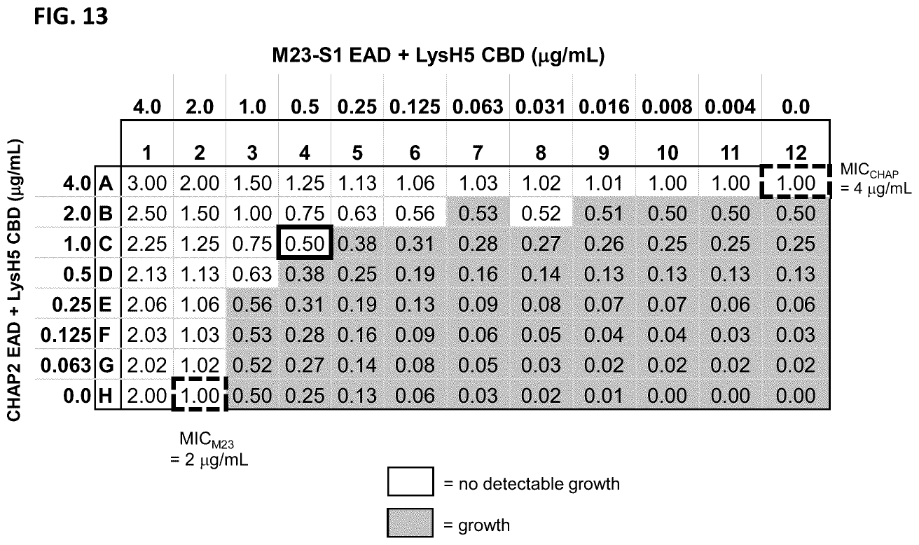

Results: M23-S1 EAD+LysH5 CBD and CHAP2 EAD+LysH5 CBD chimeric CWHs, which each demonstrated high activity and selectivity for S. aureus, were tested in combination for synergistic effects against S. aureus in a checkerboard assay. The M23-S1 EAD+LysH5 CBD chimera was Protein A in the assay (dilutions in columns 1-11), while the CHAP2 EAD+LysH5 CBD chimera was Protein B (dilutions in rows A-G). The results of the assay are shown in FIG. 13, in which each table entry corresponds to the FIC index value within the corresponding well of the 96-well plate. Using the results of row H, which contained the M23-S1 EAD+LysH5 CBD chimera alone, the MIC for this protein (MIC_{M23}) was calculated to be 2 μg/mL (see well H2, outlined in a dotted line, in FIG. 13). The MIC for the CHAP2 EAD+LysH5 CBD chimera (MIC_{CHAP}) was similarly calculated from the results of column 12, which contained this chimera alone, yielding an MIC of 4 μg/mL (see well A12, outlined in a dotted line, in FIG. 13). The inventors found that certain combinations of concentrations for the two chimeric CWHs yielded synergistic activity, as defined by a lack of detectable S. aureus growth and an FIC index less than or equal to 0.5. See, e.g., well C4 in FIG. 13, outlined in a solid black line, which had an FIC index of 0.5 and no detectable growth: this well corresponded to a concentration of 0.5 μg/mL of the M23-S1-containing chimera and 1.0 μg/mL of the CHAP2-containing chimera. These results demonstrate that combinations of these proteins can be used to elicit enhanced, synergistic activity against S. aureus.

### Example 10: Identification of Additional Novel M23 Enzymatically Active Domains

Given the strong anti-Staphylococcus activity of the M23-S1 EAD in the foregoing illustrative chimeras, a broader screening of phage tail proteins with a C-terminal M23 protease domain was undertaken, focusing on bacteriophages that can infect Staphylococcus sp. and prophages contained within Staphylococcus sp. genomes. In total, 244 proteins were identified. Bioinformatic analysis using SMART and CDD identified the specific amino acids encoding the M23 EADs. These M23 EAD sequences were compared and dereplicated resulting in a set of 120 non-redundant sequences. A computational comparison of all sequences vs. all sequences was then performed to calculate amino acid similarity for each pair-wise combination. Using an 80% amino acid identity cutoff, these 120 sequences were divided into 36 groups falling within 11 families. The families and groups of sequences identified among the 120 sequences are shown in Table 16 below. Table 1 above provides the amino acid sequences of each of the 120 M23 EAD sequences.

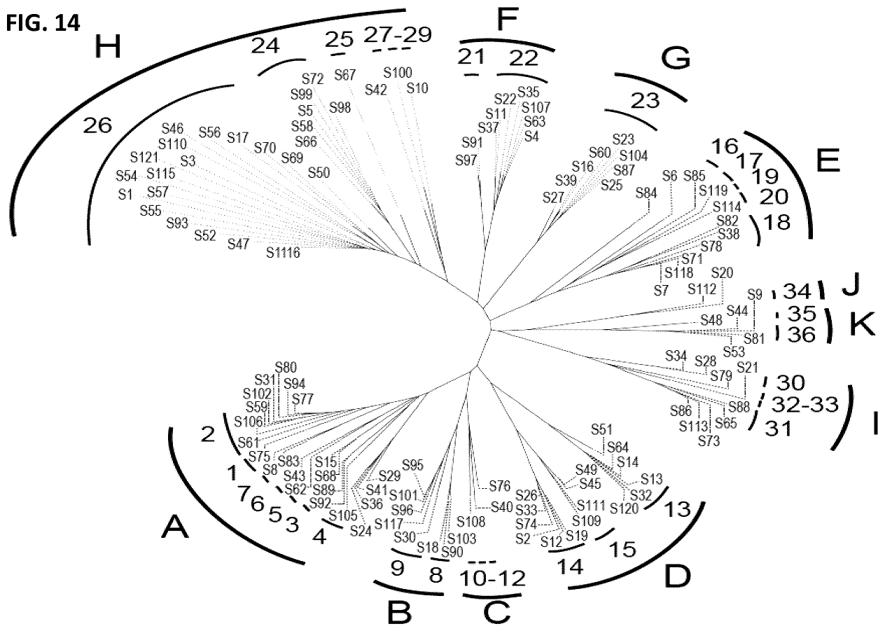

A multiple sequence alignment of the 120 non-redundant M23 sequences was generated using MUSCLE (www.ebi.ac.uk/Tools/msa/muscle/). This alignment was visualized as an unrooted phylogenetic tree employing standard parameters with the Interactive Tree of Life online tool (academic.oup.com/nar/article/49/W1/W293/6246398), as shown in FIG. 14.

TABLE 16
M23 EAD Sequence Families and Groups.

| Family | Group | Sequences |
|---|---|---|
| A | 1 | S8, S83 |
| | 2 | S75, S61, S106, S59, S31, S102, S80, S77, S94 |
| | 3 | S105 |
| | 4 | S29, S41, S24, S36 |
| | 5 | S89 |
| | 6 | S15, S68 |
| | 7 | S43, S62 |
| B | 8 | S103, S18, S90 |
| | 9 | S30, S117, S96, S95, S101 |
| C | 10 | S108 |
| | 11 | S40 |
| | 12 | S76 |
| D | 13 | S51, S64, S13, S14, S120, S32 |
| | 14 | S19, S2, S12, S74, S26, S33 |
| | 15 | S45, S49, S109, S111 |
| E | 16 | S84 |
| | 17 | S6 |
| | 18 | S78, S71, S7, S118, S38, S82 |
| | 19 | S85 |
| | 20 | S114, S119 |
| F | 21 | S91, S97 |
| | 22 | S11, S37, S22, S107, S4, S63, S35 |
| G | 23 | S27, S60, S23, S104, S25, S87, S16, S39 |
| H | 24 | S66, S58, S98, S72, S5, S99 |
| | 25 | S67 |
| | 26 | S50, S17, S69, S70, S56, S110, S46, S47, S3, |
| | | S121, S57, S1, S115, S54, S93, S55, S116, S52 |
| | 27 | S42 |
| | 28 | S10 |
| | 29 | S100 |
| I | 30 | S28, S34 |
| | 31 | S73, S86, S113, S65, S88 |
| | 32 | S21 |
| | 33 | S79 |
| J | 34 | S20, S112 |
| K | 35 | S48 |
| | 36 | S9, S44, S53, S81 |

### Example 11: Construction of Chimeras Comprising Representative M23 EADs

To test the activity level of the novel M23 sequences identified in Example 10, and their ability to function within chimeric cell wall hydrolases, chimeric enzymes were constructed from 73 representative M23 EADs from each of the 36 identified groups by linking these novel M23 EADs to the LysH5 CBD. The remaining 47 novel M23 EADs identified in Example 10 each have greater than or equal to 95% amino acid sequence identity to at least one of the 73 representative M23 EADs used to generate chimeric proteins.

Sources for EADs and CBDs. Chimeras were constructed using 73 representative novel M23 EADs from the 36 groups identified in Example 10. The chimeras comprised each of the EADs linked to the cell wall binding domain (CBD) from LysH5.

Construction of chimeric cell wall hydrolases. DNA sequences of the EADs and CBDs were codon optimized and synthesized. Individual enzymatic domains including up to 50 aa upstream and downstream were synthesized with NdeI/SpeI sites. Individual cell wall binding domains including up to 25 aa upstream and downstream were synthesized with SpeI/HindIII sites. The chimeric lysins were constructed by ligating both an enzymatic domain and a cell wall binding domain into the NdeI/HindIII sites of pET24a (+).

Protein production and purification. Expression vectors containing the cloned M23Sxx EAD+LysH5 CBD chimeric proteins were transformed into BL21 E. coli cells, protein expression was induced, and cell lysate was isolated for each chimeric enzyme.

### Example 12: Numerous M23 Chimeras Exhibit S. aureus Antimicrobial Activity

Cell lysates comprising the chimeras of Example 11 were tested for lytic activity against S. aureus using a turbidity reduction assay. The activity of each enzyme was calculated as (−ΔOD₆₀₀/min)×1000.

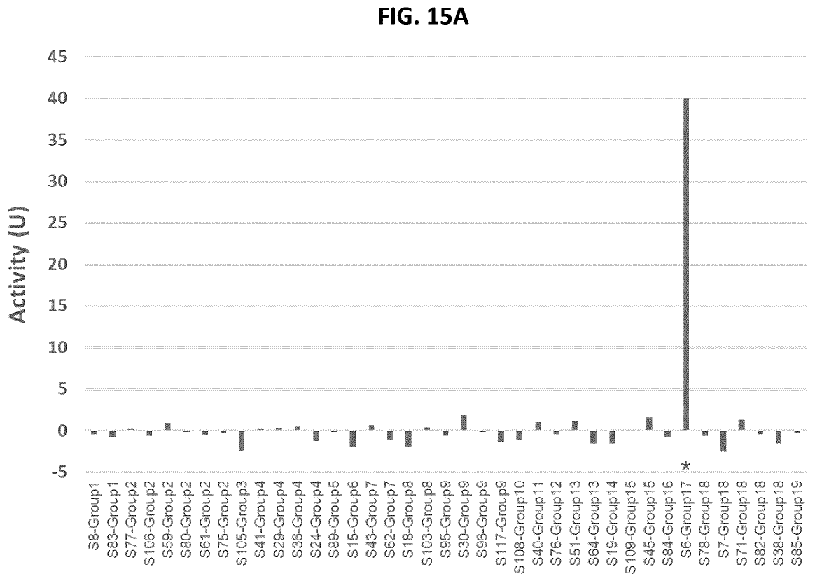

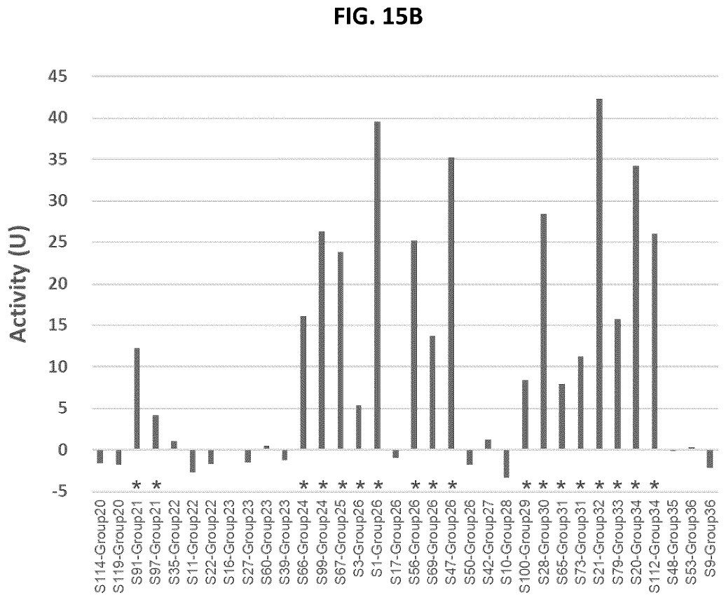

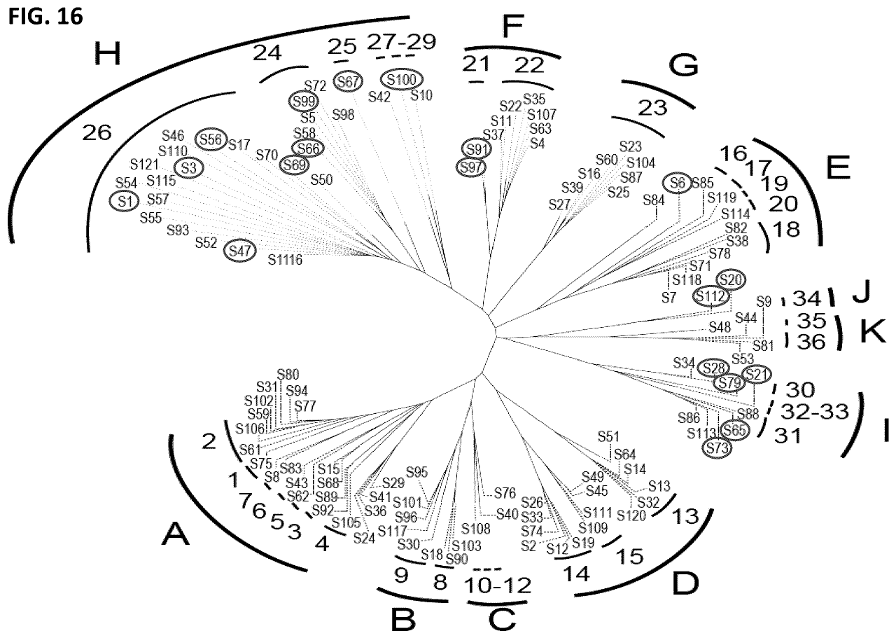

As shown in FIG. 15A-15B, 19 of the 73 chimeric proteins demonstrated antimicrobial activity against S. aureus in this assay. These 19 chimeric proteins contained EADs from 11 of the 36 groups delineated in Example 10. Strikingly, all EADs tested from groups 17, 21, 24, 25, 29, 30, 31, 32, 33, and 34 displayed S. aureus antimicrobial activity in chimeric proteins in combination with the LysH5 CBD. See FIG. 16, showing the EADs that had activity against S. aureus in this assay. In group 26, 6 out of 8 EADs (including M23-S1) displayed S. aureus antimicrobial activity in this assay. The remaining two EADs in group 26 may exhibit activity under other assay conditions.

### Example 13: The M23-S66 EAD+LysH5 CBD Chimera Exhibits Strong S. aureus-Specific Lytic Activity

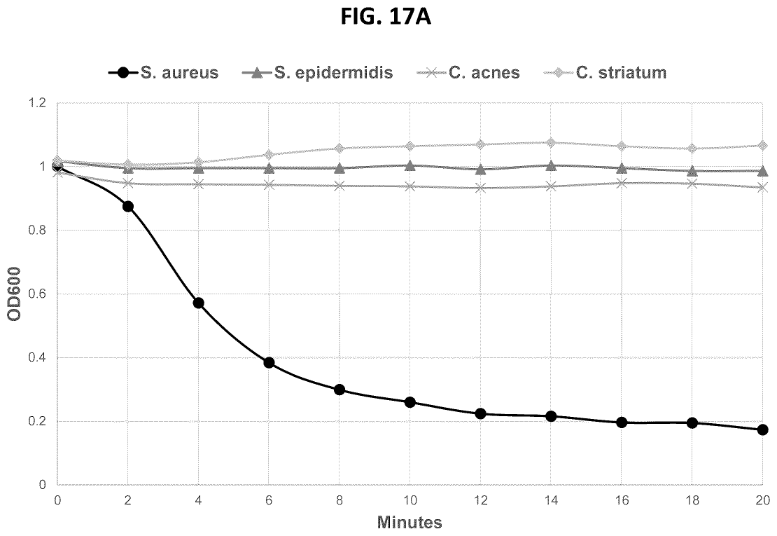

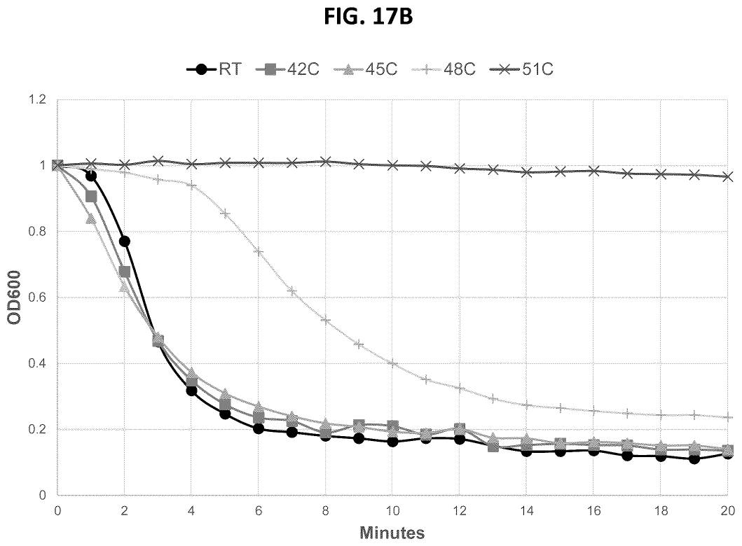

The M23-S66 EAD+LysH5 CBD chimeric protein (SEQ ID NO: 146) was selected for additional confirmatory testing. First, purified M23-S66 EAD+LysH5 CBD chimeric protein was assayed in turbidity reduction assays for antimicrobial activity against S. aureus, S. epidermidis, C. acnes, and C. striatum. S. epidermidis, C. acnes, and C. striatum are common commensal bacteria found on the skin and represent part of a healthy skin microbiome. Like the M23-S1 EAD+LysH5 CBD chimeric protein, the M23-S66 EAD+LysH5 CBD chimera displayed strong, selective lytic activity against S. aureus (FIG. 17A).

The thermostability of the M23-S66 EAD+LysH5 CBD chimera was also tested in turbidity reduction assays as described in the foregoing examples. Aliquots of the purified enzyme were incubated for 30 minutes at the indicated temperatures and then added to S. aureus in turbidity reduction assays. The protein retained activity until 51° C. (FIG. 17B).

### Example 14: Identification of Sequence Motifs Common to Active M23 EADs

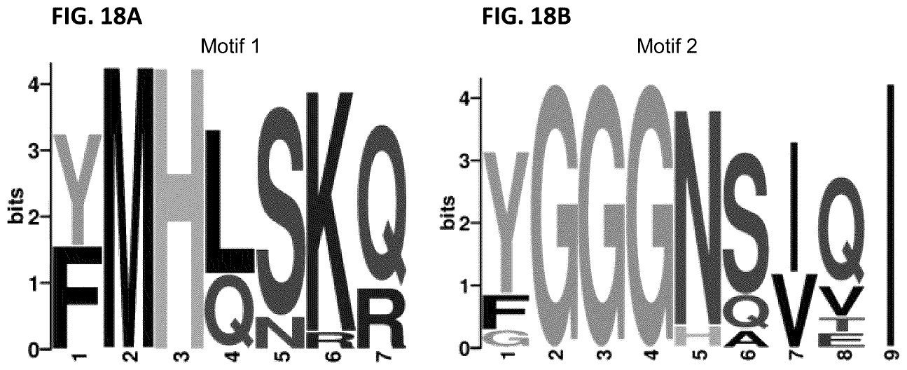

The M23 EADS identified as having S. aureus antimicrobial activity in Example 12 were analyzed to identify any sequence motifs enriched among the active proteins. Two sequence motifs were identified using STREME that are strongly enriched in M23 EADs from Example 12 with S. aureus antimicrobial activity relative to those without activity. Table 17 below provides the motif sequences and enrichment among M23 EADs identified with activity (positives) and among M23 EADs without activity (negatives). FIG. 18A-18B show the motif logos. Together, motifs 1 and 2 are found in 10 out of the 11 sequence groups that were found to have S. aureus activity in Example 12. These motifs were only identified in 1 out of 25 sequence groups that did not have detected activity in Example 12.

TABLE 17
Motif sequences, enrichment, and M23 EAD
sequence groups comprising these motifs.

| Motif | | | Found in |
|---|---|---|---|
| # | Sequence | Enrichment | Groups |
| 1 | Y/F M H L/Q S/N K/R Q/R | 16/19 positives | 21, 22, 24, 25, |
| | (SEQ ID NO: 147) | 3/54 negatives* | 26, 29, 30, 31, |
| | | | 32, 33 |
| 2 | Y/F/G G G G N/H S/Q/A | 11/19 positives | 21, 24, 25, 26, |
| | I/V X I (SEQ ID NO: 148) | 1/54 negatives** | 34 |

*2 negatives come from Group 26
**1 negative comes from Group 26

### Example 15: Construction of Chimeras Comprising Lysostaphin-Related M23 EADs

Chimeras were generated that comprised the LysH5 CBD along with M23 EADs from lysostaphin and lysostaphin-related proteins: Lss (Lysostaphin) from Staphylococcus simulans (GenBank Acc. No.: M15686.1); ALE-1 from Staphylococcus capitis (GenBank Acc. No.: D86328.1); and LytM from Staphylococcus aureus (GenBank Acc. No.: L77194.1). See Table 13.

Construction of chimeric cell wall hydrolases. DNA sequences of the EADs and CBDs were codon optimized and synthesized. Individual EADs including up to 50 aa upstream and downstream were synthesized with NdeI/SpeI sites. Individual CBDs including up to 25 aa upstream and downstream were synthesized with SpeI/HindIII sites. The chimeric lysins were constructed by ligating both an EAD and a CBD into the NdeI/HindIII sites of pET24a (+). For comparison, the wildtype mature/active forms of Lss and ALE-1 (consisting of the M23 EAD and its native CBD, while lacking the regulatory proregion) were also cloned into pET24a (+). Sequence information for the proteins, EADs, and chimeras is provided in Table 18.

TABLE 18
Sequences for lysostaphin-related proteins, EADs, and chimeras comprising.

| | SEQ | |
|---|---|---|
| Description | ID NO | Sequence |
| Lysostaphin | 149 | MKKTKNNYYTRPLAIGLSTFALASIVYGGIQNETHASEKSN |
| (Lss) full- | | MDVSKKVAEVETSKAPVENTAEVETSKAPVENTAEVETSK |
| length, mature | | APVENTAEVETSKAPVENTAEVETSKAPVENTAEVETSKA |
| protein | | PVENTAEVETSKALVQNRTALRAATHEHSAQWLNNYKKG |
| | | YGYGPYPLGINGGMHYGVDFFMNIGTPVKAISSGKIVEAG |
| | | WSNYGGGNQIGLIENDGVHRQWYMHLSKYNVKVGDYVK |
| | | AGQIIGWSGSTGYSTAPHLHFQRMVNSFSNSTAQDPMPFL |
| | | KSAGYGKAGGTVTPTPNTGWKTNKYGTLYKSESASFTPN |
| | | TDIITRTTGPFRSMPQSGVLKAGQTIHYDEVMKQDGHVWV |
| | | GYTGNSGQRIYLPVRTWNKSTNTLGVLWGTIK |
| ALE-1 full- | 150 | MDTNRKFTLVKSLSIGLGTFLVGSVFLTVNDEASASTKVD |
| length, mature | | APKVEQEAPAKADAPKVEQEAPAKADAPKVEQEAPAKVD |
| protein | | APKVEQEAPAKVDAPKVEQEAPAKADAPKVEQKRTFVRE |
| | | AAQSNHSASWLNNYKKGYGYGPYPLGINGGNHYGVDFF |
| | | MNVGTPVRAISDGKIVEAGWTNYGGGNEIGLVENDGVHR |
| | | QWYMHLSKFNVKVGDRVKAGQIIGWSGSTGYSTAPHLHF |
| | | QRMTNSFSNNTAQDPMPFLKSAGYGSNSTSSSNNNGYKTN |
| | | KYGTLYKSESASFTANTDIITRLTGPFRSMPQSGVLRKGLTI |
| | | KYDEVMKQDGHVWVGYNTNSGKRVYLPVRTWNESTGEL |
| | | GPLWGTIK |
| LytM protein | 151 | MKKLTAAAIATMGFATFTMAHQADAAETTNTQQAHTQM |
| | | STQSQDVSYGTYYTIDSNGDYHHTPDGNWNQAMFDNKEY |
| | | SYTFVDAQGHTHYFYNCYPKNANANGSGQTYVNPATAG |
| | | DNNDYTASQSQQHINQYGYQSNVGPDASYYSHSNNNQAY |
| | | NSHDGNGKVNYPNGTSNQNGGSASKATASGHAKDASWL |
| | | TSRKQLQPYGQYHSGGAHYGVDYAMPENSPVYSLTDGTV |
| | | VQAGWSNYGGGNQVTIKEANSNNYQWYMHNNRLTVSA |
| | | GDKVKAGDQIAYSGSTGNSTAPHVHFQRMSGGIGNQYAV |
| | | DPTSYLQSR |
| Lysostaphin | 152 | MAATHEHSAQWLNNYKKGYGYGPYPLGINGGMHYGVDF |
| M23 EAD | | FMNIGTPVKAISSGKIVEAGWSNYGGGNQIGLIENDGVHR |
| | | QWYMHLSKYNVKVGDYVKAGQIIGWSGSTGYSTAPHLHF |
| | | QRMVNSFSNSTAQDPMPFLKSAGYGKAGGTVTPTPNT |
| ALE-1 M23 | 153 | MSNHSASWLNNYKKGYGYGPYPLGINGGNHYGVDFFMN |
| EAD | | VGTPVRAISDGKIVEAGWTNYGGGNEIGLVENDGVHRQW |
| | | YMHLSKFNVKVGDRVKAGQIIGWSGSTGYSTAPHLHFQR |
| | | MTNSFSNNTAQDPMPFLKSAGYGSNSTSSS |
| LytM M23 | 154 | MHAKDASWLTSRKQLQPYGQYHSGGAHYGVDYAMPENS |
| EAD | | PVYSLTDGTVVQAGWSNYGGGNQVTIKEANSNNYQWYM |
| | | HNNRLTVSAGDKVKAGDQIAYSGSTGNSTAPHVHFQRMS |
| | | GGIGNQYAVDPTSYLQSR |
| LysH5 CBD- | 155 | MAATHEHSAQWLNNYKKGYGYGPYPLGINGGMHYGVDF |
| Lss EAD | | FMNIGTPVKAISSGKIVEAGWSNYGGGNQIGLIENDGVHR |
| Chimera | | QWYMHLSKYNVKVGDYVKAGQIIGWSGSTGYSTAPHLHF |
| | | QRMVNSFSNSTAQDPMPFLKSAGYGKAGGTVTPTPNTTSS |
| | | NDSSASSNTVKPVASAWKRNKYGTYYMEESARFTNGNQP |
| | | ITVRKVGPFLSCPVGYQFQPGGYCDYTEVMLQDGHVWVG |
| | | YTWEGQRYYLPIRTWNGSAPPNQILGDLWGEIS |
| LysH5 CBD- | 156 | MSNHSASWLNNYKKGYGYGPYPLGINGGNHYGVDFFMN |
| ALE-1 EAD | | VGTPVRAISDGKIVEAGWTNYGGGNEIGLVENDGVHRQW |
| Chimera | | YMHLSKFNVKVGDRVKAGQIIGWSGSTGYSTAPHLHFQR |
| | | MTNSFSNNTAQDPMPFLKSAGYGSNSTSSSTSSNDSSASSN |
| | | TVKPVASAWKRNKYGTYYMEESARFTNGNQPITVRKVGP |
| | | FLSCPVGYQFQPGGYCDYTEVMLQDGHVWVGYTWEGQR |
| | | YYLPIRTWNGSAPPNQILGDLWGEIS |
| LysH5 CBD- | 157 | MHAKDASWLTSRKQLQPYGQYHSGGAHYGVDYAMPENS |
| LytM EAD | | PVYSLTDGTVVQAGWSNYGGGNQVTIKEANSNNYQWYM |
| Chimera | | HNNRLTVSAGDKVKAGDQIAYSGSTGNSTAPHVHFQRMS |
| | | GGIGNQYAVDPTSYLQSRTSSNDSSASSNTVKPVASAWKR |
| | | NKYGTYYMEESARFTNGNQPITVRKVGPFLSCPVGYQFQP |
| | | GGYCDYTEVMLQDGHVWVGYTWEGQRYYLPIRTWNGSA |
| | | PPNQILGDLWGEIS |

Protein production and purification. The inducible expression vectors containing the chimeric and native cell wall hydrolases were chemically transformed into BL21 E. coli for downstream protein expression and purification. Proteins were expressed and purified as described in Example 2.

### Example 16: Lysostaphin-Related M23 Chimeras Show Strong Lytic Activity Against S. aureus and Improved Selectivity Compared to Native Proteins

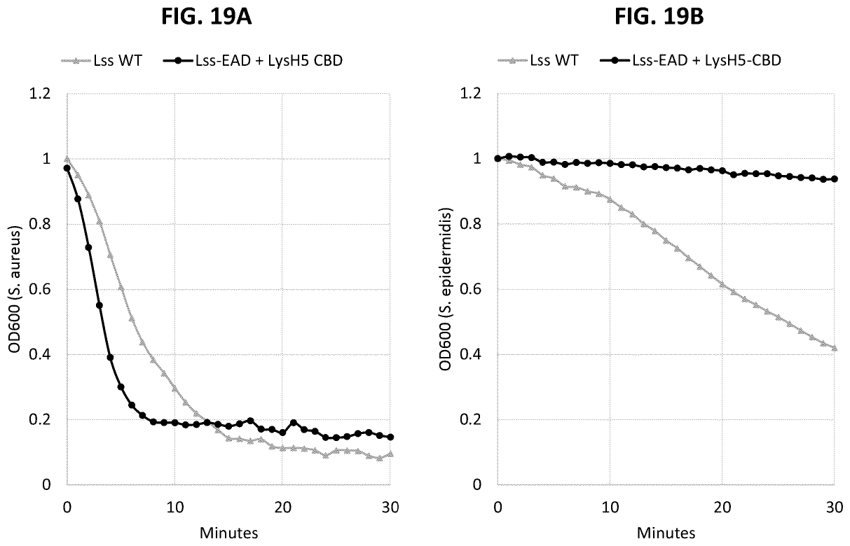

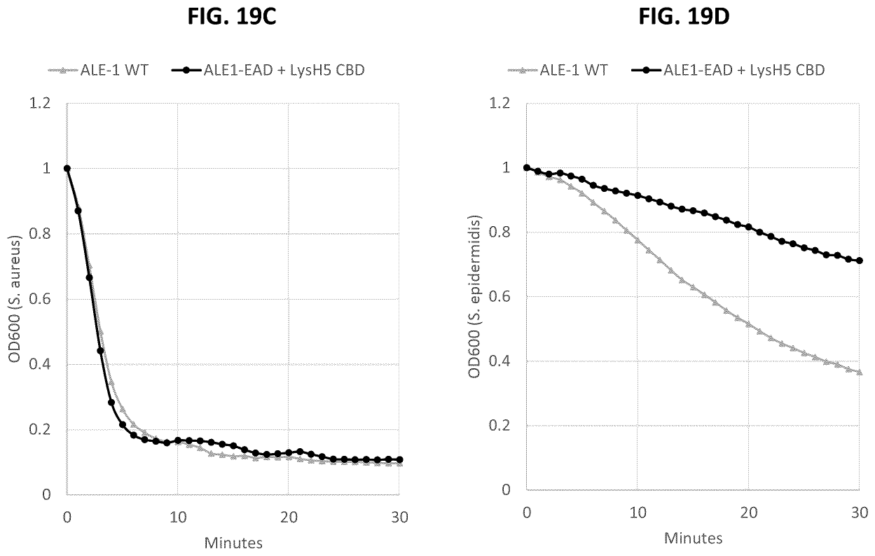

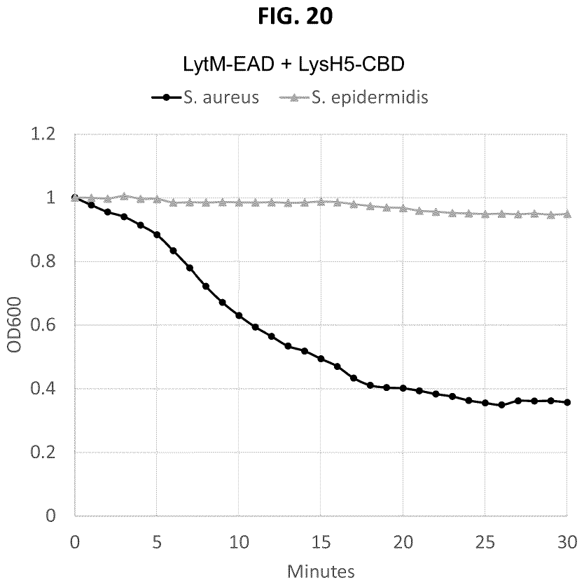

Purified Lss-EAD+LysH5-CBD (SEQ ID NO: 155) and ALE-1-EAD+LysH5-CBD (SEQ ID NO: 156) chimeric proteins were assayed in turbidity reduction assays for activity against S. aureus and S. epidermidis and compared to purified native Lss and ALE-1 that was similarly tested against S. aureus and S. epidermidis. All proteins were tested at 12 μg/mL. In both cases, the chimeric proteins showed very strong lytic activity against S. aureus that was stronger or equal to the native protein (FIG. 19A, 19C). At the same time, the chimeric proteins had weak or undetectable lytic activity against S. epidermidis that was significantly lower than the native protein (FIG. 19B, 19D). These data demonstrate that the replacement of the native CBD of Lss and ALE-1 with the LysH5 CBD resulted in chimeric proteins exhibiting significantly increased selectivity for S. aureus over S. epidermidis. Note that since LytM is an autolysin, it does not have an endolysin-like native architecture that could be tested in an activity comparison. However, purified LytM-EAD+LysH5 CBD chimeric protein, tested at 12 μg/mL, similarly exhibited strong, selective activity against S. aureus (FIG. 20).

### Example 17: Chimeric Protein of the Disclosure Retains Activity in Two Topical Hydrogel Formulations

Chimeric enzymes for topical skin applications must retain enzymatic function within formulations appropriate for topical application. Proteins tend to be more challenging to formulate than small molecules since their complex structures are more likely to suffer from issues like unfolding, degradation/aggregation, oxidative damage, and temperature/pH sensitivity.

As a representative example of a chimeric enzyme of the disclosure, the M23-S1 EAD+LysH5 CBD chimeric enzyme was formulated in two example hydrogel formulations and then tested for S. aureus-selective antimicrobial activity. Formulation #1 was a hyaluronic-acid based hydrogel, while formulation #2 was a hydroxymethylcellulose-based hydrogel. A hyaluronic acid-based hydrogel combines the hydrating effects of hyaluronic acid with the beneficial microbiome effects of the chimeric enzyme and can be used to treat Staphylococcus infection while promoting skin hydration. The M23-S1 EAD+LysH5 CBD chimeric enzyme was added to each formulation (4 μg/mL) and stored for 7 days at room temperature. These enzyme formulations were then tested for antimicrobial activity against S. aureus and S. epidermidis using a quantitative killing assay. Approximately 1×10⁶ cells of each strain were incubated with formulation with and without M23-S1 EAD+LysH5 CBD (4 μg/mL) for 2 hours. Serial dilutions were plated at the 2h time point to quantify the number of viable CFU.

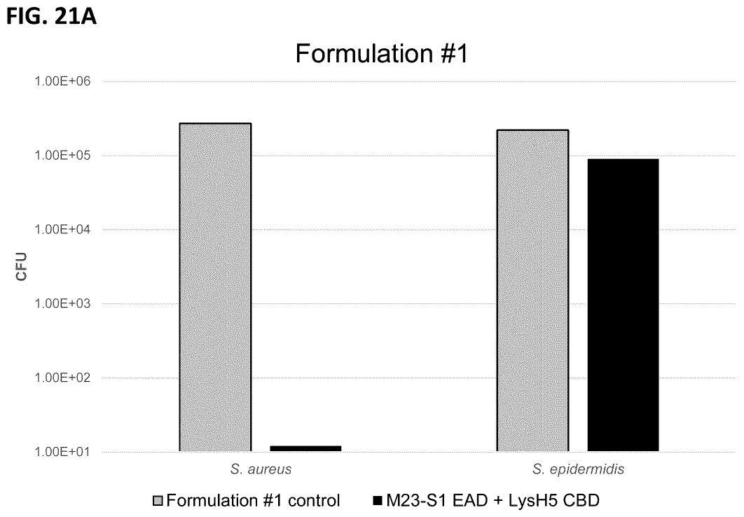

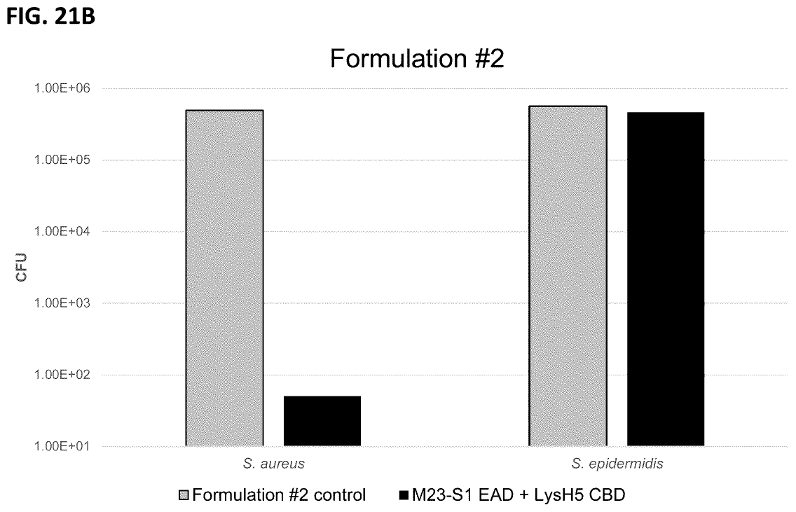

Both formulations exhibited strong, selective killing of S. aureus (FIG. 21A-21B). The number of viable CFU of S. aureus remaining after two-hour treatment with M23-S1 EAD+LysH5 CBD was reduced 3-4 orders of magnitude compared to <1 order of magnitude for S. epidermidis. These results demonstrate the ability of the M23-S1 EAD+LysH5 CBD to retain enzymatic activity in different formulations relevant for topical skin applications.

### REFERENCES

The following references are incorporated herein by reference in their entireties for all purposes.
1. Bonar E, Bukowski M, Chlebicka K, Madry A, Bereznicka A, Kosecka-Strojek M, Dubin G, Miedzobrodzki J, Mak P, Wladyka B. Human skin microbiota-friendly lysostaphin. International Journal of Biological Macromolecules. 2021 Jul. 31; 183:852-60.
2. Chang Y, Ryu S. Characterization of a novel cell wall binding domain-containing Staphylococcus aureus endolysin LysSA97. Applied microbiology and biotechnology. 2017 January; 101:147-58.
3. Eichenseher F, Herpers B L, Badoux P, Leyva-Castillo J M, Geha R S, van der Zwart M, Mckellar J, Janssen F, de Rooij B, Selvakumar L, Röhrig C. Linker-Improved Chimeric Endolysin Selectively Kills Staphylococcus aureus In Vitro, on Reconstituted Human Epidermis, and in a Murine Model of Skin Infection. Antimicrobial Agents and Chemotherapy. 2022 May 17; 66 (5): e02273-21.
4. Gutiérrez D, Garrido V, Fernández L, Portilla S, Rodríguez A, Grillo M J, García P. Phage lytic protein LysRODI prevents staphylococcal mastitis in mice. Frontiers in microbiology. 2020 Jan. 23; 11:7.
5. Gutiérrez D, Rodríguez-Rubio L, Ruas-Madiedo P, Fernández L, Campelo A B, Briers Y, Nielsen M W, Pedersen K, Lavigne R, García P, Rodríguez A. Design and selection of engineered lytic proteins with Staphylococcus aureus decolonizing activity. Frontiers in Microbiology. 2021 Sep. 14; 12:723834.
6. Kost Y, Deutsch A, Mieczkowska K, Nazarian R, Muskat A, Hosgood H D, Lin J, Daily J P, Ohri N, Kabarriti R, Shinoda K. Bacterial Decolonization for Prevention of Radiation Dermatitis: A Randomized Clinical Trial. JAMA oncology. 2023 May 4.
7. Kost Y, Rzepecki A K, Deutsch A, Birnbaum M R, Ohri N, Hosgood H D, Lin J, Daily J P, Shinoda K, Mclellan B N. Association of Staphylococcus aureus Colonization With Severity of Acute Radiation Dermatitis in Patients With Breast or Head and Neck Cancer. JAMA oncology. 2023 May 4.
8. Lee C, Kim J, Son B, Ryu S. Development of advanced chimeric endolysin to control multidrug-resistant Staphylococcus aureus through domain shuffling. ACS Infectious Diseases. 2021 May 28; 7 (8): 2081-92.
9. Matsuzaki S, Rashel M, Uchiyama J, Sakurai S, Ujihara T, Kuroda M, Ikeuchi M, Tani T, Fujieda M, Wakiguchi H, Imai S. Bacteriophage therapy: a revitalized therapy against bacterial infectious diseases. Journal of infection and chemotherapy. 2005 October; 11:211-9.
10. Obeso J M, Martínez B, Rodríguez A, García P. Lytic activity of the recombinant staphylococcal bacteriophage ΦH5 endolysin active against Staphylococcus aureus in milk. International journal of food microbiology. 2008 Dec. 10; 128 (2): 212-8.
11. Vermassen A, Leroy S, Talon R, Provot C, Popowska M, Desvaux M. Cell wall hydrolases in bacteria: insight on the diversity of cell wall amidases, glycosidases and peptidases toward peptidoglycan. Frontiers in microbiology. 2019 Feb. 28; 10:331.
12. WO 2004/020635 A1
13. WO 2009/024327 A2
14. WO 2019/105936 A1
15. WO 2007/130655 A2

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS OF THE INVENTION

Notwithstanding the appended claims, the disclosure sets forth the following numbered embodiments:

1. A recombinant protein comprising an enzymatically active domain (EAD) having the sequence of any one of SEQ ID NO: 2 or 4-123, or a sequence having at least 70, 80, 90, 95, or 99% sequence identity to any one of SEQ ID NO: 2 or 4-123.

2. The recombinant protein of embodiment 1, wherein the EAD comprises the sequence motif of SEQ ID NO: 147 and/or 148.

3. A recombinant protein comprising a heterologous enzymatically active domain (EAD) comprising the sequence motif of SEQ ID NO: 147 and/or 148.

4. The recombinant protein of embodiment 3, wherein the EAD has the sequence of any one of SEQ ID NO: 2 or 4-123, or a sequence having at least 70, 80, 90, 95, or 99% sequence identity to any one of SEQ ID NO: 2 or 4-123

5. The recombinant protein of any one of embodiments 1-4, wherein the EAD comprises a sequence having at least 70, 80, 90, 95, or 99% sequence identity to CHAP2 (SEQ ID NO: 2).

6. The recombinant protein of any one of embodiments 1-4, wherein the EAD comprises the sequence of CHAP2 (SEQ ID NO: 2).

7. The recombinant protein of any one of embodiments 1-4, wherein the EAD comprises a sequence having at least 70, 80, 90, 95, or 99% sequence identity to an M23 EAD of Family E, F, H, I, or J.

8. The recombinant protein of any one of embodiments 1-4, wherein the EAD comprises an M23 EAD of Family E, F, H, I, or J.

9. The recombinant protein of any one of embodiments 1-4, wherein the EAD comprises a sequence having at least 70, 80, 90, 95, or 99% sequence identity to an M23 EAD of Family H.

10. The recombinant protein of any one of embodiments 1-4, wherein the EAD comprises an M23 EAD of Family H.

11. The recombinant protein of any one of embodiments 1-4, wherein the EAD comprises a sequence having at least 70, 80, 90, 95, or 99% sequence identity to an M23 EAD of Group 17, 21, 24, 25, 26, 29, 30, 31, 32, 33, or 34.

12. The recombinant protein of any one of embodiments 1-4, wherein the EAD comprises an M23 EAD of Group 17, 21, 24, 25, 26, 29, 30, 31, 32, 33, or 34.

13. The recombinant protein of any one of embodiments 1-4, wherein the EAD comprises a sequence having at least 70, 80, 90, 95, or 99% sequence identity to an M23 EAD of Group 17, 21, 24, 25, 29, 30, 31, 32, 33, and 34.

14. The recombinant protein of any one of embodiments 1-4, wherein the EAD comprises an M23 EAD of Group 17, 21, 24, 25, 29, 30, 31, 32, 33, and 34.

15. The recombinant protein of any one of embodiments 1-4, wherein the EAD comprises a sequence having at least 70, 80, 90, 95, or 99% sequence identity to an M23 EAD of Group

26.

16. The recombinant protein of any one of embodiments 1-4, wherein the EAD comprises an M23 EAD of Group 26.

17. The recombinant protein of any one of embodiments 1-4, wherein the EAD comprises a sequence having at least 70, 80, 90, 95, or 99% sequence identity to an M23 EAD of Group 24.

18. The recombinant protein of any one of embodiments 1-4, wherein the EAD comprises an M23 EAD of Group 24.

19. The recombinant protein of any one of embodiments 1-4, wherein the EAD comprises a sequence having at least 70, 80, 90, 95, or 99% sequence identity to M23-S1 (SEQ ID NO: 4), M23-S3 (SEQ ID NO: 6), M23-S6 (SEQ ID NO: 9), M23-S20 (SEQ ID NO: 23), M23-S21 (SEQ ID NO: 24), M23-S28 (SEQ ID NO: 31), M23-S47 (SEQ ID NO: 50), M23-S56 (SEQ ID NO: 59), M23-S65 (SEQ ID NO: 68), M23-S66 (SEQ ID NO: 69), M23-S67 (SEQ ID NO: 70), M23-S69 (SEQ ID NO: 72), M23-S73 (SEQ ID NO: 76), M23-S79 (SEQ ID NO: 82), M23-S91 (SEQ ID NO: 94), M23-S97 (SEQ ID NO: 99), M23-S99 (SEQ ID NO: 101), M23-S100 (SEQ ID NO: 102), or M23-S112 (SEQ ID NO: 114).

20. The recombinant protein of any one of embodiments 1-4, wherein the EAD comprises M23-S1 (SEQ ID NO: 4), M23-S3 (SEQ ID NO: 6), M23-S6 (SEQ ID NO: 9), M23-S20 (SEQ ID NO: 23), M23-S21 (SEQ ID NO: 24), M23-S28 (SEQ ID NO: 31), M23-S47 (SEQ ID NO: 50), M23-S56 (SEQ ID NO: 59), M23-S65 (SEQ ID NO: 68), M23-S66 (SEQ ID NO: 69), M23-S67 (SEQ ID NO: 70), M23-S69 (SEQ ID NO: 72), M23-S73 (SEQ ID NO: 76), M23-S79 (SEQ ID NO: 82), M23-S91 (SEQ ID NO: 94), M23-S97 (SEQ ID NO: 99), M23-S99 (SEQ ID NO: 101), M23-S100 (SEQ ID NO: 102), or M23-S112 (SEQ ID NO: 114).

21. The recombinant protein of any one of embodiments 1-4, wherein the EAD comprises a sequence having at least 70, 80, 90, 95, or 99% sequence identity to M23-S1 (SEQ ID NO: 4).

22. The recombinant protein of any one of embodiments 1-4, wherein the EAD comprises M23-S1 (SEQ ID NO: 4).

23. The recombinant protein of any one of embodiments 1-4, wherein the EAD comprises a sequence having at least 70, 80, 90, 95, or 99% sequence identity to M23-S66 (SEQ ID NO: 69).

24 The recombinant protein of any one of embodiments 1-4, wherein the EAD comprises M23-S66 (SEQ ID NO: 69).

25. The recombinant protein of any one of embodiments 1-24, wherein the recombinant protein is a chimeric protein.

26. The recombinant protein of any one of embodiments 1-25, wherein the recombinant protein is a chimeric cell wall hydrolase (CWH).

27. The recombinant protein of any one of embodiments 1-26, wherein the recombinant protein comprises a cell wall binding domain (CBD).

28. The recombinant protein of any one of embodiments 1-27, wherein the recombinant protein comprises an SH3b CBD.

29. The recombinant protein of any one of embodiments 1-28, wherein the recombinant protein comprises a CBD from ALE-1 (SEQ ID NO: 127), LysA72 (SEQ ID NO: 125), LysH5 (SEQ ID NO: 124), LysSA97 (SEQ ID NO: 128), LysPALS1 (SEQ ID NO: 129), or PlySs2 (SEQ ID NO: 126), or a CBD having at least 70, 80, 90, 95, or 99% sequence identity to a CBD from ALE-1 (SEQ ID NO: 127), LysA72 (SEQ ID NO: 125), LysH5 (SEQ ID NO: 124), LysSA97 (SEQ ID NO: 128), LysPALS1 (SEQ ID NO: 129), or PlySs2 (SEQ ID NO: 126).

30. The recombinant protein of any one of embodiments 1-29, wherein the recombinant protein comprises a CBD from LysH5 (SEQ ID NO: 124), or a CBD having at least 70, 80, 90, 95, or 99% sequence identity to a CBD from LysH5 (SEQ ID NO: 124).

31. The recombinant protein of any one of embodiments 1-30, wherein the recombinant protein is active against a species of Staphylococcus.

32. The recombinant protein of any one of embodiments 1-31, wherein the recombinant protein is active against a species of Staphylococcus, and wherein the minimum inhibitory concentration (MIC) of that activity is determined via an MIC assay.

33. The recombinant protein of any one of embodiments 1-32, wherein the recombinant protein is active against a species of Staphylococcus, with an MIC less than or equal to 50 μg/mL.

34. The recombinant protein of any one of embodiments 1-33, wherein the recombinant protein is active against a species of Staphylococcus, with an MIC less than or equal to 10 μg/mL.

35. The recombinant protein of any one of embodiments 1-34, wherein the recombinant protein is active against a species of Staphylococcus, wherein the species is S. agnetis, S. argensis, S. argenteus, S. arlettae, S. aureus, S. auricularis, S. capitis, S. caprae, S. carnosus, S. chromogenes, S. cohnii, S. condimenti, S. cornubiensis, S. delphini, S. devriesei, S. edaphicus, S. epidermidis, S. equi, S. equorum, S. felis, S. fleurettii, S. gallinarum, S. haemolyticus, S. hominis, S. hyicus, S. intermedius, S. kloosii, S. lentus, S. lugdunensis, S. lutrae, S. massiliensis, S. microti, S. muscae, S. nepalensis, S. pasteuri, S. petrasii, S. pettenkoferi, S. piscifermentans, S. pseudintermedius, S. pseudoxylosus, S. rostri, S. saccharolyticus, S. saprophyticus, S. schleiferi, S. schweitzeri, S. sciuri, S. simiae, S. simulans, S. stepanovicii, S. succinus, S. vitulinus, S. warneri, or S. xylosus.

36. The recombinant protein of any one of embodiments 1-35, wherein the recombinant protein is active against Staphylococcus aureus.

37. The recombinant protein of any one of embodiments 1-36, wherein the recombinant protein is active against Staphylococcus aureus, with an MIC less than or equal to 50 μg/mL.

38. The recombinant protein of any one of embodiments 1-37, wherein the recombinant protein is active against Staphylococcus aureus, with an MIC less than or equal to 10 μg/mL.

39. The recombinant protein of any one of embodiments 1-38, wherein the protein demonstrates selective activity against one species of Staphylococcus in comparison to a second species of Staphylococcus.

40. The recombinant protein of any one of embodiments 1-39, wherein the protein demonstrates selective activity against a coagulase positive species (CoPS) of Staphylococcus over a coagulase negative species (CoNS) of Staphylococcus.

41. The recombinant protein of any one of embodiments 1-40, wherein the protein has at least 2-fold, at least 5-fold, or at least 10-fold selectivity against one species of Staphylococcus in comparison to a second species of Staphylococcus.

42. The recombinant protein of any one of embodiments 1-41, wherein the protein demonstrates selective activity against Staphylococcus aureus over Staphylococcus epidermidis.

43. The recombinant protein of any one of embodiments 1-42, wherein the protein has at least 2-fold selectivity for Staphylococcus aureus over Staphylococcus epidermidis.

44. The recombinant protein of any one of embodiments 1-43, wherein the protein has at least 10-fold selectivity for Staphylococcus aureus over Staphylococcus epidermidis.

45. The recombinant protein of any one of embodiments 1-44, wherein the protein is active against a species of Staphylococcus, and wherein the protein retains its activity after being exposed to a temperature up to 45° C.

46. The recombinant protein of any one of embodiments 1-45, wherein the protein is active against a species of Staphylococcus, and wherein the protein retains its activity after being exposed to a temperature up to 50° C.

47. The recombinant protein of any one of embodiments 1-46, wherein the protein is active within a pH range of pH 6-8.

48. The recombinant protein of any one of embodiments 1-47, wherein the protein is active within a pH range of pH 5-8.

49. A chimeric cell wall hydrolase (CWH) comprising:
a) an enzymatically active domain (EAD) having the sequence of CHAP2 (SEQ ID NO: 2), M23-S1 (SEQ ID NO: 4), or M23-S66 (SEQ ID NO: 69), or a sequence having at least 70, 80, 90, 95, or 99% sequence identity to the sequence of CHAP2 (SEQ ID NO: 2), M23-S1 (SEQ ID NO: 4), or M23-S66 (SEQ ID NO: 69); and
b) a cell wall binding domain (CBD) from ALE-1 (SEQ ID NO: 127), LysA72 (SEQ ID NO: 125), LysH5 (SEQ ID NO: 124), LysSA97 (SEQ ID NO: 128), LysPALS1 (SEQ ID NO: 129), or PlySs2 (SEQ ID NO: 126), or a CBD having at least 70, 80, 90, 95, or 99% sequence identity to a CBD from ALE-1 (SEQ ID NO: 127), LysA72 (SEQ ID NO: 125), LysH5 (SEQ ID NO: 124), LysSA97 (SEQ ID NO: 128), LysPALS1 (SEQ ID NO: 129), or PlySs2 (SEQ ID NO: 126).

50. A chimeric cell wall hydrolase (CWH) comprising:
a) an enzymatically active domain (EAD) comprising a sequence having at least 70, 80, 90, 95, or 99% sequence identity to the sequence of CHAP2 (SEQ ID NO: 2); and
b) a cell wall binding domain (CBD) comprising a sequence having at least 70, 80, 90, 95, or 99% sequence identity to the CBD from LysH5 (SEQ ID NO: 124).

51. A chimeric cell wall hydrolase (CWH) comprising:
a) an enzymatically active domain (EAD) having the sequence of CHAP2 (SEQ ID NO: 2); and
b) a cell wall binding domain (CBD) from LysH5 (SEQ ID NO: 124).

52. A chimeric cell wall hydrolase (CWH) comprising:
a) an enzymatically active domain (EAD) comprising a sequence having at least 70, 80, 90, 95, or 99% sequence identity to the sequence of M23-S1 (SEQ ID NO: 4); and
b) a cell wall binding domain (CBD) comprising a sequence having at least 70, 80, 90, 95, or 99% sequence identity to the CBD from LysH5 (SEQ ID NO: 124).

53. A chimeric cell wall hydrolase (CWH) comprising:
a) an enzymatically active domain (EAD) having the sequence of M23-S1 (SEQ ID NO: 4); and
b) a cell wall binding domain (CBD) from LysH5 (SEQ ID NO: 124).

54. A chimeric cell wall hydrolase (CWH) comprising:
a) an enzymatically active domain (EAD) comprising a sequence having at least 70, 80, 90, 95, or 99% sequence identity to the sequence of M23-S66 (SEQ ID NO: 69); and
b) a cell wall binding domain (CBD) comprising a sequence having at least 70, 80, 90, 95, or 99% sequence identity to the CBD from LysH5 (SEQ ID NO: 124).

55. A chimeric cell wall hydrolase (CWH) comprising:
a) an enzymatically active domain (EAD) having the sequence of M23-S66 (SEQ ID NO: 69); and
b) a cell wall binding domain (CBD) from LysH5 (SEQ ID NO: 124).

56. A chimeric cell wall hydrolase (CWH) comprising:
a) an enzymatically active domain (EAD) from lysostaphin or a lysostaphin-related protein, or a sequence having at least 70, 80, 90, 95, or 99% sequence identity thereto; and
b) a cell wall binding domain (CBD) from LysH5 (SEQ ID NO: 124), or a CBD having at least 70, 80, 90, 95, or 99% sequence identity to a CBD from LysH5 (SEQ ID NO: 124).

57. A chimeric cell wall hydrolase (CWH) comprising:
a) an enzymatically active domain (EAD) having the sequence of the Lss EAD (SEQ ID NO: 152), or a sequence having at least 70, 80, 90, 95, or 99% sequence identity to the sequence of the Lss EAD (SEQ ID NO: 152); and
b) a cell wall binding domain (CBD) from LysH5 (SEQ ID NO: 124), or a CBD having at least 70, 80, 90, 95, or 99% sequence identity to a CBD from LysH5 (SEQ ID NO: 124).

58. A chimeric cell wall hydrolase (CWH) comprising:
a) an enzymatically active domain (EAD) having the sequence of the ALE-1 EAD (SEQ ID NO: 153), or a sequence having at least 70, 80, 90, 95, or 99% sequence identity to the sequence of the ALE-1 EAD (SEQ ID NO: 153; and
b) a cell wall binding domain (CBD) from LysH5 (SEQ ID NO: 124), or a CBD having at least 70, 80, 90, 95, or 99% sequence identity to a CBD from LysH5 (SEQ ID NO: 124).

59. A chimeric cell wall hydrolase (CWH) comprising:
a) an enzymatically active domain (EAD) having the sequence of the LytM EAD (SEQ ID NO: 154), or a sequence having at least 70, 80, 90, 95, or 99% sequence identity to the sequence of the LytM EAD (SEQ ID NO: 154); and
b) a cell wall binding domain (CBD) from LysH5 (SEQ ID NO: 124), or a CBD having at least 70, 80, 90, 95, or 99% sequence identity to a CBD from LysH5 (SEQ ID NO: 124).

60. The chimeric CWH of any one of embodiments 49-59, wherein the chimeric CWH is active against a species of Staphylococcus.

61. The chimeric CWH of any one of embodiments 49-60, wherein the chimeric CWH is active against a species of Staphylococcus, and wherein the minimum inhibitory concentration (MIC) of that activity is determined via an MIC assay.

62. The chimeric CWH of any one of embodiments 49-61, wherein the chimeric CWH is active against a species of Staphylococcus, with an MIC less than or equal to 50 μg/mL.

63. The chimeric CWH of any one of embodiments 49-62, wherein the chimeric CWH is active against a species of Staphylococcus, with an MIC less than or equal to 10 μg/mL.

64. The chimeric CWH of any one of embodiments 49-63, wherein the chimeric CWH is active against a species of Staphylococcus, wherein the species is S. agnetis, S. argensis, S. argenteus, S. arlettae, S. aureus, S. auricularis, S. capitis, S. caprae, S. carnosus, S. chromogenes, S. cohnii, S. condimenti, S. cornubiensis, S. delphini, S. devriesei, S. edaphicus, S. epidermidis, S. equi, S. equorum, S. felis, S. fleurettii, S. gallinarum, S. haemolyticus, S. hominis, S. hyicus, S. intermedius, S. kloosii, S. lentus, S. lugdunensis, S. lutrae, S. massiliensis, S. microti, S. muscae, S. nepalensis, S. pasteuri, S. petrasii, S. pettenkoferi, S. piscifermentans, S. pseudintermedius, S. pseudoxylosus, S. rostri, S. saccharolyticus, S. saprophyticus, S. schleiferi, S. schweitzeri, S. sciuri, S. simiae, S. simulans, S. stepanovicii, S. succinus, S. vitulinus, S. warneri, or S. xylosus.

65. The chimeric CWH of any one of embodiments 49-64, wherein the chimeric CWH is active against Staphylococcus aureus.

66. The chimeric CWH of any one of embodiments 49-65, wherein the chimeric CWH is active against Staphylococcus aureus, with an MIC less than or equal to 50 μg/mL.

67. The chimeric CWH of any one of embodiments 49-66, wherein the chimeric CWH is active against Staphylococcus aureus, with an MIC less than or equal to 10 μg/mL.

68. The chimeric CWH of any one of embodiments 49-67, wherein the CWH demonstrates selective activity against one species of Staphylococcus in comparison to a second species of Staphylococcus.

69 The chimeric CWH of any one of embodiments 49-68, wherein the protein demonstrates selective activity against a coagulase positive species (CoPS) of Staphylococcus over a coagulase negative species (CoNS) of Staphylococcus.

70. The chimeric CWH of any one of embodiments 49-69, wherein the CWH has at least 2-fold, at least 5-fold, or at least 10-fold selectivity against one species of Staphylococcus in comparison to a second species of Staphylococcus.

71. The chimeric CWH of any one of embodiments 49-70, wherein the CWH demonstrates selective activity against Staphylococcus aureus over Staphylococcus epidermidis.

72. The chimeric CWH of any one of embodiments 49-71, wherein the CWH has at least 2-fold selectivity for Staphylococcus aureus over Staphylococcus epidermidis.

73. The chimeric CWH of any one of embodiments 49-72, wherein the CWH has at least 10-fold selectivity for Staphylococcus aureus over Staphylococcus epidermidis.

74. The chimeric CWH of any one of embodiments 49-73, wherein the CWH has improved selectivity for Staphylococcus aureus over Staphylococcus epidermidis compared to a native protein comprising the EAD or the CBD comprised by the CWH.

75. The chimeric CWH of any one of embodiments 49-74, wherein the CWH is active against a species of Staphylococcus, and wherein the CWH retains its activity at a temperature up to 45° C.

76. The chimeric CWH of any one of embodiments 49-75, wherein the CWH is active against a species of Staphylococcus, and wherein the CWH retains its activity at a temperature up to 50° C.

77. The chimeric CWH of any one of embodiments 49-76, wherein the CWH is active within a pH range of pH 6-8.

78. The chimeric CWH of any one of embodiments 49-77, wherein the CWH is active within a pH range of pH 5-8.

79. A composition comprising a first and a second recombinant protein, wherein each recombinant protein comprises: a recombinant protein according to any one of embodiments 1-48 or a chimeric CWH according to any one of embodiments 49-78.

80. A composition comprising a first and a second recombinant protein, wherein each recombinant protein comprises: an enzymatically active domain (EAD) having the sequence of any one of SEQ ID NO: 2 or 4-123, or a sequence having at least 70, 80, 90, 95, or 99% sequence identity to any one of SEQ ID NO: 2 or 4-123.

81. The composition of embodiment 80, wherein each recombinant protein comprises: a cell wall binding domain (CBD) from ALE-1 (SEQ ID NO: 127), LysA72 (SEQ ID NO: 125), LysH5 (SEQ ID NO: 124), LysSA97 (SEQ ID NO: 128), LysPALS1 (SEQ ID NO: 129), or PlySs2 (SEQ ID NO: 126), or a CBD having at least 70, 80, 90, 95, or 99% sequence identity to a CBD from ALE-1 (SEQ ID NO: 127), LysA72 (SEQ ID NO: 125), LysH5 (SEQ ID NO: 124), LysSA97 (SEQ ID NO: 128), LysPALS1 (SEQ ID NO: 129), or PlySs2 (SEQ ID NO: 126).

82. The composition of embodiment 80 or 81, wherein the first recombinant protein comprises:
a) an enzymatically active domain (EAD) having the sequence of CHAP2 (SEQ ID NO: 2); and
b) a cell wall binding domain (CBD) from LysH5 (SEQ ID NO: 124).

83. The composition of any one of embodiments 80-82, wherein the second recombinant protein comprises:
a) an enzymatically active domain (EAD) having the sequence of M23-S1 (SEQ ID NO: 4) or M23-S66 (SEQ ID NO: 69); and
b) a cell wall binding domain (CBD) from LysH5 (SEQ ID NO: 124).

84. The composition of any one of embodiments 79-83, wherein the composition exhibits synergistic activity against a species of Staphylococcus compared to the composition with either the first or the second recombinant protein alone.

85. The composition of any one of embodiments 79-84, wherein the composition exhibits synergistic activity against Staphylococcus aureus compared to the composition with either the first or the second recombinant protein alone.

86. The composition of any one of embodiments 79-85, wherein the composition comprises 0.1-10.0 μg/mL of each recombinant protein.

87. The composition of any one of embodiments 79-86, wherein the composition comprises 0.25-2.0 μg/mL of each recombinant protein.

88. The composition of any one of embodiments 79-87, wherein the composition comprises about 1 μg/mL of the first recombinant protein and about 0.5 μg/mL of the second recombinant protein.

89. A topical formulation comprising a recombinant protein according to any one of embodiments 1-48, a chimeric CWH according to any one of embodiments 49-78, or a composition according to any one of embodiments 79-88.

90. The formulation of embodiment 89, wherein the formulation is a hydrogel, lotion, cream, or gel-cream.

91. The formulation of embodiment 89 or embodiment 90, wherein the formulation is a hydrogel.

92 The formulation of any one of embodiments 89-91, wherein the formulation comprises a humectant.

93. The formulation of any one of embodiments 89-92, wherein the formulation comprises a humectant selected from the list consisting of: aloe vera, betaine, butylene glycol, caprylyl glycol, dimethicone, fructose, glucomannan, glucose, glycerin, glyceryl glucoside, honey, hyaluronic acid, lactic acid, panthenol, polyethylene glycol, propylene glycol, propanediol, sodium hyaluronate, sodium lactate, sodium pyrrolidone carboxylic acid, sorbitol, and urea.

94 The formulation of any one of embodiments 89-93, wherein the formulation comprises 0.1-50% w/v humectant.

95. The formulation of any one of embodiments 89-94, wherein the formulation comprises 0.5-10% w/v humectant.

96. The formulation of any one of embodiments 89-95, wherein the formulation comprises a cellulose polymer.

97. The formulation of any one of embodiments 89-96, wherein the formulation comprises a cellulose polymer selected from the list consisting of: hydroxyethyl cellulose, methylcellulose, hydroxy methylcellulose, carboxymethyl cellulose, microcrystalline cellulose, ethyl cellulose, sodium carboxymethyl cellulose, hydroxypropyl cellulose, and cellulose acetate.

98. The formulation of any one of embodiments 89-97, wherein the formulation comprises 0.5-10% w/v of a cellulose polymer.

99. The formulation of any one of embodiments 89-98, wherein the formulation comprises 1-5% w/v of a cellulose polymer.

100. The formulation of any one of embodiments 89-99, wherein the formulation comprises a salt.

101. The formulation of any one of embodiments 89-100, wherein the formulation comprises a salt selected from the list consisting of: calcium chloride, Dead Sea salt, Epsom salt, Himalayan pink salt, magnesium chloride, sea salt, and sodium chloride.

102. The formulation of any one of embodiments 89-101, wherein the formulation comprises 10-500 mM of a salt.

103. The formulation of any one of embodiments 89-102, wherein the formulation comprises 50-250 mM of a salt.

104. The formulation of any one of embodiments 89-103, wherein the formulation comprises a buffer.

105. The formulation of any one of embodiments 89-104, wherein the formulation comprises a buffer selected from the list consisting of: 4-(2-hydroxyethyl)-1-piperazineethanesulfonic acid, acetic acid, ammonium acetate, boric acid, citric acid, glycine, phosphoric acid, potassium hydroxide, potassium phosphate, sodium acetate, sodium bicarbonate, sodium borate, sodium carbonate, sodium citrate, sodium dihydrogen phosphate, sodium hydrogen phosphate, sodium hydroxide, sodium phosphate, sodium tetraborate, tris(hydroxymethyl)aminomethane, and trisodium phosphate.

106. The formulation of any one of embodiments 89-105, wherein the formulation comprises 5-50 mM of a buffer.

107. The formulation of any one of embodiments 89-106, wherein the formulation comprises a surfactant.

108. The formulation of any one of embodiments 89-107, wherein the formulation comprises a surfactant selected from the list consisting of: ceteareth-20, cocamidopropyl betaine, coco-glucoside, decyl glucoside, decyl polyglucose, disodium laureth sulfosuccinate, glycereth-26, lauryl glucoside, lauryl polyglucose, sodium cocoyl glutamate, sodium cocoyl isethionate, sodium laureth sulfate, and sodium lauryl sulfate.

109. The formulation of any one of embodiments 89-108, wherein the formulation comprises 0.1-20% w/v of a surfactant.

110. The formulation of any one of embodiments 89-109, wherein the formulation comprises 1-10% w/v of a surfactant.

111. The formulation of any one of embodiments 89-110, wherein the formulation comprises a free amino acid.

112. The formulation of any one of embodiments 89-111, wherein the formulation comprises a free amino acid selected from the list consisting of: alanine, arginine, cysteine, glutamine, glycine, histidine, lysine, methionine, proline, serine, and threonine.

113. The formulation of any one of embodiments 89-112, wherein the formulation comprises 10-250 mM of a free amino acid.

114. The formulation of any one of embodiments 89-113, wherein the formulation comprises an oil.

115. The formulation of any one of embodiments 89-114, wherein the formulation comprises an oil selected from the list consisting of: argan oil, avocado oil, baobab oil, camellia oil, carrot seed oil, coconut oil, evening primrose oil, grapeseed oil, hemp seed oil, jojoba oil, macadamia nut oil, marula oil, mineral oil, olive oil, pomegranate seed oil, raspberry seed oil, rosehip seed oil, squalane oil, sunflower seed oil, sweet almond oil, and tamanu oil.

116. The formulation of any one of embodiments 89-115, wherein the formulation comprises 0.1-20% w/v of an oil.

117. The formulation of any one of embodiments 89-116, wherein the formulation comprises an alcohol.

118. The formulation of any one of embodiments 89-117, wherein the formulation comprises an alcohol selected from the list consisting of: cetyl alcohol, ethyl alcohol, isopropyl alcohol, and stearyl alcohol.

119. The formulation of any one of embodiments 89-118, wherein the formulation comprises 0.1-20% w/v of an alcohol.

120. The formulation of any one of embodiments 89-119, wherein the formulation comprises 1-10% w/v of an alcohol.

121. The formulation of any one of embodiments 89-120, wherein the formulation comprises glycerol.

122. The formulation of any one of embodiments 89-121, wherein the formulation comprises 0.5-50% w/v glycerol.

123. The formulation of any one of embodiments 89-122, wherein the formulation comprises 1-30% w/v glycerol.

124. The formulation of any one of embodiments 89-123, wherein the formulation comprises 1-5% w/v glycerol.

125. The formulation of any one of embodiments 89-124, wherein the formulation comprises petrolatum.

126. The formulation of any one of embodiments 89-125, wherein the formulation comprises 0.1-20% w/v petrolatum.

127. The formulation of any one of embodiments 89-126, wherein the formulation is thermostable at 45° C. for at least four weeks.

128. The formulation of any one of embodiments 89-127, wherein the formulation is thermostable at 45° C. for at least two months.

129. The formulation of any one of embodiments 89-128, wherein the formulation is thermostable at 50° C. for at least two months.

130. The formulation of any one of embodiments 89-129, wherein the formulation is active within a pH range of 6-8

131. The formulation of any one of embodiments 89-130, wherein the formulation is active within a pH range of 5-8.

132. A method of treating a condition associated with Staphylococcus, the method comprising the step of: administering a composition comprising a recombinant protein according to any one of embodiments 1-48, a composition comprising a chimeric CWH according to any one of embodiments 49-78, a composition according to any one of embodiments 79-88, or a formulation according to any one of embodiments 89-131.

133. The method of embodiment 132, wherein the composition or formulation is administered topically, enterally, or parenterally.

134. The method of embodiment 132 or embodiment 133, wherein the composition or formulation is administered topically.

135. The method of any one of embodiments 132-134, wherein the composition or formulation is administered 1-4 times every 1-7 days.

136. The method of any one of embodiments 132-135, wherein the composition or formulation is administered once or twice daily.

137. The method of any one of embodiments 132-136, wherein the composition or formulation is administered for a period of 1-12 weeks.

138. The method of any one of embodiments 132-137, wherein the composition or formulation is administered until resolution of symptoms.

139. The method of any one of embodiments 132-138, wherein the condition is associated with a species of Staphylococcus selected from: S. agnetis, S. argensis, S. argenteus, S. arlettae, S. aureus, S. auricularis, S. capitis, S. caprae, S. carnosus, S. chromogenes, S. cohnii, S. condimenti, S. cornubiensis, S. delphini, S. devriesei, S. edaphicus, S. epidermidis, S. equi, S. equorum, S. felis, S. fleurettii, S. gallinarum, S. haemolyticus, S. hominis, S. hyicus, S. intermedius, S. kloosii, S. lentus, S. lugdunensis, S. lutrae, S. massiliensis, S. microti, S. muscae, S. nepalensis, S. pasteuri, S. petrasii, S. pettenkoferi, S. piscifermentans, S. pseudintermedius, S. pseudoxylosus, S. rostri, S. saccharolyticus, S. saprophyticus, S. schleiferi, S. schweitzeri, S. sciuri, S. simiae, S. simulans, S. stepanovicii, S. succinus, S. vitulinus, S. warneri, and S. xylosus.

140. The method of any one of embodiments 132-139, wherein the condition is associated with Staphylococcus aureus.

141. The method of any one of embodiments 132-140, wherein the condition is an infection by a species of Staphylococcus.

142. The method of any one of embodiments 132-141, wherein the condition is overgrowth of a species of Staphylococcus.

143. The method of any one of embodiments 132-142, wherein the condition is dry skin, itchy skin, and/or red skin.

144. The method of any one of embodiments 132-142, wherein the condition is atopic dermatitis.

145. The method of any one of embodiments 132-142, wherein the condition is wound infection.

146. The method of any one of embodiments 132-142, wherein the condition is acute radiation dermatitis.

147. The method of any one of embodiments 132-142, wherein the condition is a chronic wound.

148. The method of any one of embodiments 132-147, wherein the method comprises a step of applying a second topical formulation after administration of the composition comprising a recombinant protein according to any one of embodiments 1-48, composition comprising a chimeric CWH according to any one of embodiments 49-78, composition according to any one of embodiments 79-88, or formulation according to any one of embodiments 89-131.

149. The method of embodiment 148, wherein the second topical formulation is a hydrating formulation.

150. The method of embodiment 148 or embodiment 149, wherein the second topical formulation restores the skin barrier.

151. The method of any one of embodiments 148-150, wherein the second topical formulation is a cream or lotion.

152. The method of any one of embodiments 148-150, wherein the second topical formulation is applied within 60 minutes of application of the composition comprising a recombinant protein according to any one of embodiments 1-48, composition comprising a chimeric CWH according to any one of embodiments 49-78, composition according to any one of embodiments 79-88, or formulation according to any one of embodiments 89-131.

153. The method of any one of embodiments 148-150, wherein the second topical formulation is applied within 15 minutes of application of the composition comprising a recombinant protein according to any one of embodiments 1-48, composition comprising a chimeric CWH according to any one of embodiments 49-78, composition according to any one of embodiments 79-88, or formulation according to any one of embodiments 89-131.

1. A composition, system, or method as disclosed herein.


SEQUENCE LISTING (157 sequences)

SEQ ID NO: 1 - protein, 372 aa, Sciuriunavirus SscM1
MKTQKQVVDYIKSLEGKGWDWDNYAGWQCFDTANYHAYFAMGMSLAGEGAKDIPYKNDFTGKADIYNNTPEFLAQPGDIVVWTSPTFGGGYGHVASVISATLNTITVIEQNWLGGGISKTEVATRRSHSYDPSMVFIRPKYAKSSTVTESKPTTVKPVTKPKAPATSNSSSGAKAIVKGKVPPKTLSNSKGAYFRAKADKYGVNICKLQNGKYYPTNEVYQAGYDQFYIFEIKNGWARVYSNSNNGWVWFERLRVVEIFKNKTVAKAKATVAKAKKKVTPKKKTPAPKLKLAIGKKVPKGTIKVQKKPILQAKADSAGVTITTRNGTLKNEKYKPGQIFYVYEVTKDGYCRVYSPDNNGYVWHERLIITKVY

SEQ ID NO: 2 - protein, 92 aa, synthetic construct
DWDNYAGWQCFDTANYHAYFAMGMSLAGEGAKDIPYKNDFTGKADIYNNTPEFLAQPGDIVVWTSPTFGGGYGHVASVISATLNTITVIEQN

SEQ ID NO: 3 - protein, 2066 aa, Triavirus StauST3982
MNEKVEGMTLELKLDHLGVQEGMKGLKRQLGVVNSEMKANLSAFDKSEKSMEKYQARIKGLNDRLKVQKKMYSQVEDELKQVNANYQKAKSSVKDVEKAYLKLVEANKKEKLALDKSKEALKSSNTELKKAENQYKRTNQRKQDAYQKLKQLRDAEQKLKNSNQATTAQLKRASDAVQKQSAKHKALVEQYKQEGNQVQKLKVQNDNLSKSNDKIESSYAKTNTKLKQTEKEFNDLNNTIKNHSANVAKAETAVNKEKAALNNLERSIDKASSEMKTFNKEQMIAQSHFGKLASQADVMSKKFSSIGDKMTSLGRTMTMGVSTPITLGLGAALKTSADFEGQMSRVGAIAQASSKDLKSMSNQAVDLGAKTSKSANEVAKGMEELAALGFNAKQTMEAMPGVISAAEASGAEMATTATVMASAINSFGLKASDANHVADLLARSANDSAADIQYMGDALKYAGTPAKALGVSIEDTSAAIEVLSNSGLEGSQAGTALRASFIRLANPSKSTAKEMKKLGIHLSDAKGQFVGMGELIRQFQDNMKGMTREQKLATVATIVGTEAASGFLALIEAGPDKINSYSKSLKNSNGESKKAADLMKDNLKGALEQLGGAFESLAIEVGKDLTPMIRAGAEGLTKLVDGFTHLPGWVRKASVGLALFGAAIGPAVLAGGLLIRTVGSAAKGYASLNRRIAENTILSNTNSKAMKSLGLQTLFLGSTTGKTSKGFKGLAGAMMFNLKPINVLKNSAKLAILPFKLLKNGLGLAAKSLFAVSGGARFAGVALRFLTGPIGATITAITIAYKVFKTAYDRVEWFRNGINGLGETIKFFGGKIIGGAVRKLGEFKNYLGSIGKSFKEKFSKDMKDGYKSLSDDDLLKVGVNKFKGFMQTMGTASKKASDTVKVLGKGVSKETEKALEKYVHYSEENSRIMEKVRLNSGQISEDKAKKLLKIETDLSNNLIAEIEKRNKKELEKTQELIDKYSAFDEQEKQNILTRTKEKNDLRIKKEQELNQKIKELKEKALSDGQISENERKEIEKLENQRRDITVKELSKTEKEQERILVRMQRNRNAYSIDEASKAIKEAEKARKARKKEVDKQYEDDVIAIKNNVNLSKSEKDKLLAIADQRHKDEVRKAKSKKDAVVDVVKKQNKDIDKEMDLSSGRVYKNTEKWWNGLKSWWSNFREDQKKKSDKYAKEQEETARRNRENIKKWFGNAWDGVKSKTGEAFSKMGRNANHFGGEMKKMWSGIKGIPSKLSSGWSSAKSSVGYHTKAIANSTGKWFGKAWQSVKSTTGSIYNQTKQKYSDASDKAWAHSKSIWRGTSKWFSNAYKSAKGWLTDMANKSRAKWDNISSTAWSNAKSVWKGTSKWFSNSYKSLKDWTGDMYSRAHDRFDAISSSAWSNAKSVFNGFRKWLSKTYDWIRDIGKDMGRAAADLGKNVANKAIGGLNSMIGGINKISKAITDKNLIKPIPTLSTGTLAGKGVATDNSGALTQPTFAVLNDRGSGNAPGGGVQEVIHRADGTFHAPQGRDVVVPLGVGDSVINANDTLKLQRMGVLPKFHGGTKKKKWMEQVTENLGKKAGDFGSKAKNTAHNIKKGAEEMVEAAGDKIKDGASWLGDKIGDVWDYVQHPGKLVNKVMSGLNINFGGGANATVKLAKGAYSLLKKKLVDKVKSWFEDFGGGGDGSYLFEYPIWQRFGRYTGGLNFNGGRHYGIDFGMPSGTNVYAVKGGIADKVWTDYGGGNSIQIKTGANEWNWYMHLSKQLARQGQRIKAGQLIGKSGATGNFVRGAHLHFQLMQGSHPGNDTAKDPEKWLKSLKGSGVRSGSGVNKAASAWAGDIRRAAKRMGVNVTSGDVGNIISLIQHESGGNAGITQSSSLRDINVLQGNPAKGLLQYIPQTFRHYAVRGHNNIYSGYDQLLAFFNNRYWRSQFNPRGGWSPSGPRRYANGGLITKHQLAEVGEGDKQEMVIPLTRRKRAIQLTEQVMRIIGMDGKPNNITVNNDTSTVEKLLKQIVMLSDKGNKLTDALIQTVSSQDNNLGSNDAIRGLEKILSKQSGHRANANNYMGGLTN

SEQ ID NO: 4 - protein, 102 aa, synthetic construct
GRHYGIDFGMPSGTNVYAVKGGIADKVWTDYGGGNSIQIKTGANEWNWYMHLSKQLARQGQRIKAGQLIGKSGATGNFVRGAHLHFQLMQGSHPGNDTAKDP

SEQ ID NO: 5 - protein, 97 aa, synthetic construct
PFHEGVDFPFVYQEVRTPMGGRLTRMPFMSGGYGNYVKITSGVIDMLFAHLKNFSKSPPSGTMVKPGDVVGLTGNTGFSTGPHLHFEMRRNGRHFDP

SEQ ID NO: 6 - protein, 102 aa, synthetic construct
GRHYGIDFQMPTGTNIYAVKGGIADKVWTDYGGGNSIQIKTGANEWNWYMHLSKQLARQGQRIKAGQLIGKSGATGNFVRGAHLHFQLMRGSHPGNDTAVDP

SEQ ID NO: 7 - protein, 100 aa, synthetic construct
GVHHGVDYDTPTGTPIRTPMGGRVRSWYDNYGGGKAITVQKGRTFLWFMHLSEQLRRTGEQIKAGQLIGKSGNTGSMTNYRHLHFQVNQGGESNRYSTDP

SEQ ID NO: 8 - protein, 102 aa, synthetic construct
GRHYGMDFGMPAGTKVYAVKGGTVDNVWYDYGGGNSIQIKTGPGEWNWYMHLSKQLVRLGEHIRTGQLIAESGATGAFCKGAHLHFQLMRGDHPGNDTAIDP

SEQ ID NO: 9 - protein, 100 aa, synthetic construct
GHHYGIDYATPYGTLITAPTSGTVSRQYNQYGGLIARLVSGIYAQYFLHLSEVLKTGRVEQGEPIARTGNSGQWTTGPHLHYQVESPFGAELTNRNTINP

SEQ ID NO: 10 - protein, 100 aa, synthetic construct
GHHYGIDFGAPYGTTINATNSGQLGELHNFGGGLVARLLTGQFTLFFMHLSKILKHGKVQAGEPIAKTGNSGNWTTGPHLHFQVEKGRHNDITNQNTVNP

SEQ ID NO: 11 - protein, 104 aa, synthetic construct
RAHHGLDINYKYDKVYSTLSGIATGSSGWNGGFGQNMWIRAKGGLEAIYGHLHKLAFHGKKRVKPGTYLGISGGDPGRDGQNAGSSTGPHLHYEMRRNGVPFDP

SEQ ID NO: 12 - protein, 106 aa, synthetic construct
GVHHGRDITSATINGSPIKAARSGIVTFKGWTGGGNTLSIFDGKNTYTYMHMKNPARVVKGQRVKAGQIVGNVGTTYDRRLGGFSTGPHLHVQANLGKTPSGTFMN

SEQ ID NO: 13 - protein, 98 aa, synthetic construct
GKHYGMDFGMLPGTNVYAVAGGKASRVWHDYGGGNSIEVDLGGGLTNWYMHLQKQLVKQGQRIKAGDLIAKSGNTGAFTAGTGHLHFQLNKNGVPKDP

SEQ ID NO: 14 - protein, 100 aa, synthetic construct
GVHHGVDYDTPVGTPIRTPMAGRVRSWYDNYGGGNAITVSKGKTFLWFMHLSKQLKKTGEQVKAGQLIGKSGNTGSMTNYRHLHFQVNQGGESNSNSVEP

SEQ ID NO: 15 - protein, 97 aa, synthetic construct
PFHEGVDFPFVYQEVRTPMGGRLTRMPFMSGGYGNYVKITSGVIDMLFAHLKNFSKSPPSGTMVKPGDVVGLTGNTGFSTGPHLHFEMRRNGRHFNP

SEQ ID NO: 16 - protein, 95 aa, synthetic construct
PFHEGLDFDYIYEPVPSTINGRAQVMPFHNGGYGKWVKIVKGTLEVIYAHLSKYKVKTGQQVRVGQTVGISGNTGFSTGPHLHYEMRWNGRHRDP

SEQ ID NO: 17 - protein, 95 aa, synthetic construct
PFHEGLDFDYIYEPVPSTINGRAQVMPVHNGGYGKWVKIVKGALEVIYAHLSKYKVKTGQQVRVGQTVGISGNTGFSTGPHLHYEMRWNGRHRDP

SEQ ID NO: 18 - protein, 104 aa, synthetic construct
RAHHGLDIGYPYGTKVFSTTGGTATASKGWNGGFGNMVSVKSGTMETIYGHLSKHAFSGSKKVKPGDLLGLSGGDPSKQGREAGSSTGAHLHYEMRWGGVPKDP

SEQ ID NO: 19 - protein, 102 aa, synthetic construct
GKHYGIDFGMPTGTKIRALTAGKISQAGPVAGGGGNQITLDEPGGKWFQWYMHMSKVIAKKGQRVNAGDVIGLSGNTGNSTTPHLHIQRMKGYPSNETAINP

SEQ ID NO: 20 - protein, 102 aa, synthetic construct
GKHYGMDFGMPTGTPIYAVKGGVADKVWTDYGGGNSVQIKTGANEWNWYMHLSKQIAKQGQKIRAGQLIGKSGATGNFVRGAHLHFQLMRGSHAGNDTAVNP

SEQ ID NO: 21 - protein, 99 aa, synthetic construct
AHPGIDLPYHYEKVQTPLGGTIKTGEMPGGFGHYLRVMAKPYDAYFGHLSKWLVKDGQHVSPSDAIAISGNTGASTGPHLHFEMNKHGFGANTGHSIDP

SEQ ID NO: 22 - protein, 97 aa, synthetic construct
PFHEGVDFPFVYQEVRTPMGGRLTRMPFMSGGYGNYVKITSGVIDMLFAHLKNFSKSPPSGTMVKPGDVVGLTGNTGFSTGPHLHFEMRRNGQHFDP

SEQ ID NO: 23 - protein, 101 aa, synthetic construct
TPHYGVDYASSVGSPIKASRGGIVLSSGWSNYGGGNQVVIYNPKLNKTFTYMHMLSDLRVKKGQQVVAGQMIGRMGNTGNSTGPHLHFQVNEGKGFTAAGT

SEQ ID NO: 24 - protein, 104 aa, synthetic construct
TGHAGIDYAASVGTKIPSPLDGTVIKSWQSPWGGGNETQVYDGSKYTHIFMHQSKRGVSTGDKVHQGQIIGLTGNTGNSTGPHLHWQVNKGKGFLNNHPDSINP

SEQ ID NO: 25 - protein, 98 aa, synthetic construct
GVHHGIDYDTPVGTPIRTPMGGRVRSWYDNYGGGKAITVQQGKTFLWFMHLSQQLRKTGEQIKAGQLIGKSGNTGSMTNYRHLHFQVNQGGEANRYSV

SEQ ID NO: 26 - protein, 102 aa, synthetic construct
GRHYGVDFGMPTGTKIKALTDGKISQAGAVAGGGGNQITLDEPGGKWYQWYMHMSKIIAKKGQKVSAGDVIGLSGNTGNSTTPHLHIQRMKGYPSNETAVDP

SEQ ID NO: 27 - protein, 95 aa, synthetic construct
YPHMGVDLNYVYDKLYSTHSGIATGKTGYNGGFGNHMSIKSGIYEIIYGHMSKLAWTGSKRVHPGSYLGVSGNTGMSSGPHLHYEMRKNGVPINP

SEQ ID NO: 28 - protein, 102 aa, synthetic construct
SRHYGVDFGMPTGTKIKALTDGKISQAGAVAGGGGNQITLDEPGGKWYQWYMHMSKIIAKKGQKVSAGDVIGLSGSTGNSTTPHLHIQRMKGYPSNETAVNP

SEQ ID NO: 29 - protein, 97 aa, synthetic construct
PFHEGVDFPFVYQTVRTPMGGRLTRMPFMSGGYGNYVKITSGAIDMLFAHLKDFSKSPPSGSTVKPGDVVGLTGNTGFSTGPHLHFEMRRNGRHFDP

SEQ ID NO: 30 - protein, 102 aa, synthetic construct
GRHYGIDFGMPTGTKIKALTDGTVTQAGAVAGGGGNQVTLKEPGGKWYQWYMHMSKILTKKGAKVKAGDLLGLSGNTGNSTTPHLHIQRMKGYPSNETAVNP

SEQ ID NO: 31 - protein, 104 aa, synthetic construct
GGHAGIDYAVPGGTKIPSPIDGEVIQRWFSPFGGGNETQVYDGSKYTHIFMHQSKQIAKKGQRISQGDIIGLVGNTGNSFGDHLHWQVNKGKGFRNNHPDSINP

SEQ ID NO: 32 - protein, 95 aa, synthetic construct
YPHKGVDLNYVYDRLYSTHAGTATGSMGYNGGFGNHMRIRSGIYEIIYGHMSKLNWTGSKKVRPGSFLGISGNTGMSSGPHLHYEMRKNGRAIDP

SEQ ID NO: 33 - protein, 100 aa, synthetic construct
AHPGIDLPYHYEKVQSTTSGIARTKDTGNVGFGHHIVVEGKPYDVIYGHLSKWLVKNGEHVHPGTVLGISGSTGSSTGPHLHYEMNKHGFGSMTGHSIDP

SEQ ID NO: 34 - protein, 103 aa, synthetic construct
RPHFGLDINYKHDKVYSTMSGTAKTFNGWSGGFGRHVEITNGNLKSIYGHLHKLAFNGTKKVRPGTFLGISGGDPREDGQNAGSSTGLHLHYEMQRNGRAFDP

SEQ ID NO: 35 - protein, 95 aa, synthetic construct
PFHEGLDFDYIYEPVPSTINGRAQVMPFHNGGYGKWVKIVKGALEVIYAHLSKYKVKTGQQVRVGQTVGISGNTGFSTGPHLHYEMRWNGRHRDP

SEQ ID NO: 36 - protein, 97 aa, synthetic construct
PFHEGVDFPFVYQTVRTPMGGRLTRMPFMDGGYGNYVKITSGAIDMLFAHLKNFSKSPPSGSTVKPGDVVGLTGNTGFSTGPHLHFEMRRNGRHFDP

SEQ ID NO: 37 - protein, 104 aa, synthetic construct
GGHAGIDYAVPGGTKIPSPIDGEVIQRWFSPYGGGNETQVYDGSKYTHIFMHQSKQIAKKGQRISQGDIIGLVGNTGNSFGDHLHWQVNKGRGFRNNHPDSINP

SEQ ID NO: 38 - protein, 96 aa, synthetic construct
GVHHGVDYDTPVGTPIRTPMGGRVRSWYDNYGGGKAITVQKGRTFLWFMHLSEQLRRTGEQIKAGQLIGKSGNTGSMTNYRHLHFQVNQGGEANRF

SEQ ID NO: 39 - protein, 95 aa, synthetic construct
YPHMGVDLNYVYDKLYSTHGGVATGKTGYNGGFGNSMWIKSGIYEIIYGHMSKLAWTGAKKVHPGSYLGISGNTGMSSGPHLHYEMRKNGVPIDP

SEQ ID NO: 40 - protein, 100 aa, synthetic construct
GVHHGVDYDMPVGTPVRTPMAGRVRSWYDNYGGGNAITVSKGKTFLWFMHLSKQLKKTGEQVKAGQLIGKSGNTGSMTNYRHLHFQVNQGGESNSNSVEP

SEQ ID NO: 41 - protein, 100 aa, synthetic construct
GAHFGIDYGAPYGTTINATNDGNVKAIHNLGGGLVARLLTGQFTLFFMHLSKILKQGKIKAGEPMAKTGNSGQWTTGPHVHFQVERGRHDDITNRGTVNP

SEQ ID NO: 42 - protein, 102 aa, synthetic construct
GRHYGMDFAMPTGTSIKALTAGKISQAGPVAGGGGNQITLDEPGGKWFQWYMHMSKIIAKKGQRVNAGDVIGLSGNTGNSTTPHLHIQRMKGYPSNETAVNP

SEQ ID NO: 43 - protein, 99 aa, synthetic construct
AHPGIDLPYHHEPVQSTVSGKAYRKYMPNGFGNYVLVKSGNLEVFYGHLLKYLIKNGQSVRPGTKLAISGGALSDPGHGASTGMHLHYEMHQNGKPINP

SEQ ID NO: 44 - protein, 95 aa, synthetic construct
YPHMGVDLNYVYDKLYSTHNGTATAKSGYNGGFGNSMWIKSGIYEIIYGHMSKLAFNGSKKVHPGSYLGVSGNTGMSSGPHLHYEMRKNGKPIDP

SEQ ID NO: 45 - protein, 102 aa, synthetic construct
GRHYGIDFGMPIGTPVKAVADGKVSKVWNDYGGGKSIEVQVGKNLWNWYMHLSEQMAKQGQKVSAGDVIGKSGDTGNFVNGAHLHFQLNKGDHSGNDTAINP

SEQ ID NO: 46 - protein, 104 aa, synthetic construct
RPHHGLDINYPYGTKVYSTVSGKATGSKGYNGGFGNMMSVISGAIEVIYGHLSKLNWTGSKSVKPGTLLGLSGGDPARQGAGAGSSTGPHLHYEMRWNGVAKDP

SEQ ID NO: 47 - protein, 107 aa, synthetic construct
GVHHGRDITSATINGSPIKAARSGIVTFKGWTGGGNTLSIFDGKNTYTYMHMKNPARVVKGQRVKAGQIVGNVGTTHDRRLGGFSTGPHLHVQVNLGKTPSGTFMNT

SEQ ID NO: 48 - protein, 97 aa, synthetic construct
PFHEGVDFPFVYKTIRTPMGGKVARQSFMHGGYGNWVKVISGAMEMIFAHLRDFSKTPPTGKTVKAGDVIGLTGNTGFSTGPHLHFGIRKNGRDVDP

SEQ ID NO: 49 - protein, 102 aa, synthetic construct
GRHYGVDFGMPAGTNIYAVKGGIADRVWTDFGGGNSIQIKTGANEWNWYMHLSKQIARQGQRIKAGQLIGLSGATGNFVRGAHLHFQLMRGSHPGNDTAVDP

SEQ ID NO: 50 - protein, 102 aa, synthetic construct
GRHYGVDFGMDPGTNIYAVKGGIADKVWTDYGGGNSIQIKTGANEWNWYMHLSKQLVRQGQRIKAGQLIGKSGATGNFVRGAHLHFQLMQGSHPGNDTAKDP

SEQ ID NO: 51 - protein, 104 aa, synthetic construct
GAHYGLDITSANINNSAIRAAKSGVVTFKGWTGGGNTISIFDGKNTYTYMHMIRPSKLKVGSTVQAGQHVGNVGSTFGRGGRSTGPHLHVQVNKGRTPTGTFMD

SEQ ID NO: 52 - protein, 97 aa, synthetic construct
PFHEGVDFPFVYKTIRTPMGGKVARQSFMHGGYGNWVKVISGAMEMIFAHLRDFSKTPPSGKTVKAGDVIGLTGNTGFSTGPHLHFGIRKNGRDVDP

SEQ ID NO: 53 - protein, 102 aa, synthetic construct
GRHYGVDFGMPIGTNVYAVKGGKADKVWTDYGGGKSVQIKTGQGEWNWYMHLSKQIAKQGQRIKPGQLIGKSGDTGNFVRGAHLHFQLMKGSHAGNDTAKDP

SEQ ID NO: 54 - protein, 95 aa, synthetic construct
PFHEGLDFDYIYEPLPSTINGTAQVMPFMHGGYGNWVKIVQGALEVIYAHLSKHKLKTGQKVKIGDIVGISGDTGFSTGPHLHYEMRRNGRHFNP

SEQ ID NO: 55 - protein, 102 aa, synthetic construct
GRHYGIDFQMPTGTNIYAVKGGIADKVWTDYGGGNSIQIKTGANEWNWYMHLSKQLVRQGQRIKAGQLIGKSGATGNFVRGAHLHFQLMQGSHPGNDTAKDP

SEQ ID NO: 56 - protein, 106 aa, synthetic construct
GVHHGRDITSGTIDGSPIKAARSGVVTFKGWTGGGNTLSIFDGKNTYTYMHMKNPAKVVKGQRVKAGQIVGNVGTTADRSLGGFSTGSHLHVQVNLGKTPSGTFMN

SEQ ID NO: 57 - protein, 102 aa, synthetic construct
GRHYGIDFGMPTGTNVYAVKGGIADKVWTDYGGGNSIQIKTGANEWNWYMHLSKQLARQGQRIKAGQLIGKSGATGNFVRGAHLHFQLMQGSHPGNDTAKDP

SEQ ID NO: 58 - protein, 102 aa, synthetic construct
GRHYGIDFGMPTGTNIYAVKGGIADKVWTDYGGGNSIQIKTGANEWNWYMHLSKQLARQGQRIKAGQLIGKSGATGNFVRGAHLHFQLMQGSHPGNDTAKDP

SEQ ID NO: 59 - protein, 102 aa, synthetic construct
GRHYGVDFGMPAGTSIYAVKGGIADRVWTDFGGGNSIQIKTGANEWNWYMHLSKQIARQGQRIKAGQLIGLSGATGNFVKGAHLHFQLMRGSHPGNDTAVDP

SEQ ID NO: 60 - protein, 102 aa, synthetic construct
GRHYGIDFGMPSGTNVYAVKGGIADKVWTDYSGGNSIQIKTGANEWNWYMHLSKQLARQGQRIKAGQLIGKSGATGNFVRGAHLHFQLMQGSHPGNDTAKDP

SEQ ID NO: 61 - protein, 102 aa, synthetic construct
GRHYGMDFGMPAGTKIYAVKGGTVDNVWYDYGGGNSIQIKTGPGEWNWYMHLSKQLVRLGEHIRTGQLIAESGATGAYCKGAHLHFQLMRGDHPGNDTAIDP

SEQ ID NO: 62 - protein, 103 aa, synthetic construct
RPHYGLDINYKHDKVYSTMSGTARTFNGWSGGFGRHVEITNGNLKSIYGHLHKLAFNGTKKVKPGTFLGISGGDPREDGQNAGSSTGLHLHYEMQRNGRAFDP

SEQ ID NO: 63 - protein, 102 aa, synthetic construct
GKHYGIDFGMPTGTKIKALTDGKISQAGAVAGGGGNQITLDEPGGKWYQWYMHMSKIIAKKGQKVSAGDVIGLSGSTGNSTTPHLHIQRMKGYPSNETAVNP

SEQ ID NO: 64 - protein, 103 aa, synthetic construct
RPHYGLDINYKHDKVYSTMGGTATAARGWNGGFGNFVTVTSGALKSIYGHLHKLAFTGTKKVKPGTFLGISGGDPREDGQGAGSSTGLHLHYEMQRNGRPFDP

SEQ ID NO: 65 - protein, 104 aa, synthetic construct
RPHHGIDVNYPYGTKVYSTIAGKATGSHGYNCGFGNMMQIISGAIKVIYGHLSKLAFTGSKQVRPGSFLGLSGGDPARQGAGAGSSTGPHLHYEMQWNGVPKDP

SEQ ID NO: 66 - protein, 100 aa, synthetic construct
GVHHGVDYDTPIGTPIRTPMGGRVRSWYDNYGGGKAITVQKGRTFLWFMHLSEQLRRTGEQIKAGQLIGKSGNTGSMTNYRHLHFQVNQGGESNRYSTDP

SEQ ID NO: 67 - protein, 95 aa, synthetic construct
PFHEGLDFDYIYEPVPSTINGRAQVMPFHNGGYGKWVKIVKGALEVIYAHLSKYKVKTGQQVRVGQTVGISGNTGFSTGPHLHYEMRWNGMHRDP

SEQ ID NO: 68 - protein, 106 aa, synthetic construct
SGHAGIDYGAPTGTPIPSPIDGKVIQSWFSPNQPSGGNETQIWDGQKYTHIFMHQSKRKVKTGDRVRQGQIIGLVGNTGNSFGSHLHWQVNKGKGYLNNHPDSVNP

SEQ ID NO: 69 - protein, 102 aa, synthetic construct
GRHYGMDFGMPAGTSVYAVKGGTVDKLWYDYGGGNSIQIKTGPGEWNWYMHLSKQLVKLGEQFKTGQLIAKSGETGAYCKGAHLHFQLMRGDHPGNDTAIDP

SEQ ID NO: 70 - protein, 102 aa, synthetic construct
GRHYGIDFGMTPGTPVYAVKGGKARVFDDYGGGHSVEIKTGANEWNWYMHLSKQIAKTGEQIKAGQLIAKSGNTGAFTAGSGHLHFQLMQGSHPGNDTAKDP

SEQ ID NO: 71 - protein, 104 aa, synthetic construct
RAHHGLDIGYPYGTKVFSTTGGTATASKGWNGGFGNMVSVKSGTMETIYGHLSKHAFSGSKKVKPGDLLGLSGGDPSRQGREAGSSTGAHLHYEMRWGGVPKDP

SEQ ID NO: 72 - protein, 102 aa, synthetic construct
GKHYGMDFGMPTGTNIYAVKGGIADKVWTDFGGGNSVQIKTAANEWNWYMHLSKQIARQGQRIKAGQLIGKSGATGNFVRGAHLHFQLMRGGHPGNDTAVNP

SEQ ID NO: 73 - protein, 102 aa, synthetic construct
GKHYGMDFGMPTGTNVYAVKGGIADKVWTDFGGGNSVQIKTAANEWNWYMHLSKQIARQGQRIKAGQLIGKSGATGNFVRGAHLHFQLMRGGHPGNDTAVNP

SEQ ID NO: 74 - protein, 100 aa, synthetic construct
GHHYGIDFGAPFGTTINATNSGQLGELHNYGGGLVARLLTGQFTLFFMHLSKILKHGKVQAGEPIAKTGNSGHWTTGAHLHFQVEKGRHNDITNQNTINP

SEQ ID NO: 75 - protein, 102 aa, synthetic construct
GRHYGMDFGMPAGTKVYAVKGGTVDNVWYDYGGGNSIQIKTGPSEWNWYMHLSKQLVRLGEHIRTGQLIAESGATGAYCKGAHLHFQLMRGDHPGNDTAIDP

SEQ ID NO: 76 - protein, 104 aa, synthetic construct
GGHAGIDYGAATGTPIPSPIDGKVIQSWFSPNQPSGGNETQIWDGHKYTHIFMHQSKRKVKTGDRVHQGQIIGLVGDTGNSFGSHLHWQVNKGKGYLNNHPDSI

SEQ ID NO: 77 - protein, 97 aa, synthetic construct
PFHEGVDFPFIYQTVRTPMGGRLTRMPFMAGGYGNYVKITSGAIDMLFAHLKNFSKSPPSGSTVKPGDVVGLTGNTGFSTGPHLHFEMRRNGRHFDP

SEQ ID NO: 78 - protein, 103 aa, synthetic construct
RPHYGLDINYKHDKVYSTLNGIANASYGWNGGFGNMVKITSGALTAIYGHMHKLAFTGSKKVHPGSYLGISGGNPSEDGQGAGSSTGLHLHYEMQRNGVPFDP

SEQ ID NO: 79 - protein, 94 aa, synthetic construct
WPHPGIDLPYIYEPVYSTISGKAYTKEMPKGFGHYIQVKGGALDVIYGHLSKWLVKNGQKVQPGTKLGISGNTGASTGPHLHYEMHKNGKPIDP

SEQ ID NO: 80 - protein, 103 aa, synthetic construct
RPHYGLDINYKHDKVYSTMSGTAKAFTGWSGGFGNHMEVTNGNLKSIYGHLHKLAFHGTKKVKPGTFLGISGGDPREDGQGAGSSTGLHLHYEMQRNGRAFDP

SEQ ID NO: 81 - protein, 100 aa, synthetic construct
GHHYGIDFGTPYGTTINSTNDGNLKEIHNFGGGLVARLLTGQFTLFFMHLSKILKHGKVKAGEPIAKTGNSGNWTTGPHLHFQVEKGRHDTITNANTVNP

SEQ ID NO: 82 - protein, 101 aa, synthetic construct
GGHAGIDYGAPTGTPIPSPIDGKVIKSWQSPWGGGNETQVYDGNKYTHIFMHQSRRGVSAGDKVHQGEILGKVGSTGNSSGPHLHWQVNKGKGYLNNHPDS

SEQ ID NO: 83 - protein, 103 aa, synthetic construct
RPHHGLDINYKHDKVYSTMSGTAKATTGWGGGFGNHVEITNGNLKSIYGHLHKLAFHGTKKVKPGTFLGISGGDPREDGQGAGSSTGLHLHYEMQRNGQPFDP

SEQ ID NO: 84 - protein, 107 aa, synthetic construct
GAHHGRDITSGTINGSPIKAARSGVVTFKGWTGGGNTLSIFDGKNTYTYMHMKNPAKVVKGQRVKAGQIVGNVGTTADRSLGGFSTGPHLHVQVNLGKTPSGTFMNT

SEQ ID NO: 85 - protein, 100 aa, synthetic construct
GAHFGIDYGAPYGTTINATNDGVVKGIHNFGGGLVARLLTGQFTLFFMHLSKILKEGKIKAGEPMAKTGNSGHWTTGPHLHFQVEKGRHDTITNANTVDP

SEQ ID NO: 86 - protein, 104 aa, synthetic construct
RAHHGLDINYKYDKVYSTLSGMATASSGWNGGFGQNVWIRAKNGLEAIYGHLHKLQFSGKKRVKPGDLLGVSGGDPGRDGANAGSSTGPHLHYEMRKNGVPFAP

SEQ ID NO: 87 - protein, 99 aa, synthetic construct
GRHYGIDTPHQYEKIQAPTGGIVRKQYDPGGGTVAQILNGKLAQYYLHLTDVLKTGRIKKGQTFAKTGNSGANTTGPHLHTQIEDPAANALTNSNTKDP

SEQ ID NO: 88 - protein, 100 aa, synthetic construct
GKHFGIDYATPSGTTLKATNDGKVSKLHDHGGGTVAKLLSGKFTQFFMHLSNVLKTGKVKQGEGFAKTGNSGAWTTGPHLHYQVERGNSPFVTNKNTMDP

SEQ ID NO: 89 - protein, 104 aa, synthetic construct
NGHAGIDYGAATGTPIPSPIDGKVIQSWFSPNQPSGGNETQIWDGHKYTHIFMHQSKRKVKTGDRVHQGQIIGLVGDTGNSFGSHLHWQVNKGKGYLNNHPDSI

SEQ ID NO: 90 - protein, 102 aa, synthetic construct
GRHYGVDFGMPTGTKIKALTDGKISQAGAVAGGGGNQITLDEPGGKWYQWYMHMSKIIAKKGQKVSAGDVIGLSGSTGNSTTPHLHIQRMKGYPSNETAVNP

SEQ ID NO: 91 - protein, 106 aa, synthetic construct
SGHAGIDYGAPTGTPIPSPIDGKVIQSWFSPNQPSGGNETQIWDGQKYTHIFMHQSKRKVKIGDRVHQGQIIGLVGNTGNSFGSHLHWQVNKGKGYLNNHPDSVNP

SEQ ID NO: 92 - protein, 105 aa, synthetic construct
GRHDGLDIGYPYGSKLYSTMSGTGTGSKGWNGGFGNNMWLNKGGPVEAIYGHMSELAWTGKKKVKPGTYIGKSGGDPARQGAGAGQSTGPHLHYEMRWNGQPKDP

SEQ ID NO: 93 - protein, 99 aa, synthetic construct
AHPGIDLPYRYEKVQTPLGGTIKTGEMPGGFGHYLRVMAKPYDAYFGHLSKWLVKDGQHVSPGDAIAISGNTGASTGPHLHFEMNKHGFGANTGHSIDP

SEQ ID NO: 94 - protein, 100 aa, synthetic construct
GVHYGIDYDMPENTPVRTPMGGKIRNWFDNGGGGNAVTISKNGTYLWFMHLNKQLRKQGEIVKSGDLIAKSGNTGSMTNYRHLHFQVMKGSESNRAAIDP

SEQ ID NO: 95 - protein, 102 aa, synthetic construct
GRHYGIDFQMPTGTNIYAVKGGIADKVWTDYGGGNSIQIKTGANEWNWYMHLSKQLARQGQRIKAGQLIGKSGATGNFVRGAHLHFQLMQGSHPGNDTAKDP

SEQ ID NO: 96 - protein, 103 aa, synthetic construct
RPHYGLDINYKHDKVYSTMSGTAKAFTGWSGGFGNHMEVTNGNVKSIYGHLHKLAFHGTKKVKPGTFLGISGGDPREDGQGAGSSTGLHLHYEMQWNGQPKDP

SEQ ID NO: 97 - protein, 99 aa, synthetic construct
AHPGIDLPYIYEKVLTPMGGKVETRHTASGFGKHVIVRAKPYDAYFGHLSKWLVKNGQRVKPGDAIGISGDTGSSSGPHLHYEMNKHGFGSMTGHSIDP

SEQ ID NO: 98 - protein, 99 aa, synthetic construct
AHPGIDLPYIYEKVQTPLEGKVETRNTASGFGHHIIVRAKPYDAYFGHLSKWLVKNGQHVKPGDTIGISGNTGSSSGPHLHYEMNKHGFGSMTGHSIDP

SEQ ID NO: 99 - protein, 100 aa, synthetic construct
GVHYGVDYDIPENTPVRTPMGGRIRNWFDNGGGGNTVTVSKNGTYLWFMHLNKQLRKNGETVKAGDLIAKSGNTGSMTNYRHLHFQVMKGSESNSAAIDP

SEQ ID NO: 100 - protein, 102 aa, synthetic construct
GRHYGMDFGMPAGTKVYAVKGGTVDNVWYDYGGGNSIQIKTGPSEWNWYMHLSKQLVRLGEYIRTGQLIAESGATGAYCKGAHLHFQLMRGDHPGNDTAIDP

SEQ ID NO: 101 - protein, 102 aa, synthetic construct
GRHYGMDFGMPAGTKVYAVKGGTVDNVWYDYGGGNSIQIKTGPSEWNWYMHLSKQLVRLGEHIRTGQLIAESGATGAFCKGAHLHFQLMRGDHPGNDTAIDP

SEQ ID NO: 102 - protein, 97 aa, synthetic construct
GNHYGLDFGMPTGTSIKAVAGGKVSRVWNDYGGGKSIEVALGGGLTNWYMHLSKQLVKQGQKVSVGDEIAKSGATGNFVRGAHLHFQLNKNGKPQSN

SEQ ID NO: 103 - protein, 99 aa, synthetic construct
AHPGIDLPYIYEKVLTPMGGKVETRHTASGFGKHVIVRAKPYDAYFGHLSKWLVKNGQHVKPGDAIGISGNTGSSSGPHLHYEMNKHGFGSMTGHSIDP

SEQ ID NO: 104 - protein, 103 aa, synthetic construct
RPHFGLDINYKHDKVYSTMSGTARTFNGWSGGFGRHVEITNGNLKSIYGHLHKLAFNGTKKVRPGTFLGISGGDPREDGQNAGSSTGLHLHYEMQRNGRAFDP

SEQ ID NO: 105 - protein, 99 aa, synthetic construct
AHPGIDLPYRNEKVQTPLGGTIKTGEMPGGFGHYLRVMAKPYDAYFGHLSKWLVKDGQHVKPGDTIAISGSTGASTGPHLHFELNKHGFAANTGHSIDP

SEQ ID NO: 106 - protein, 102 aa, synthetic construct
GRHYGVDFGMPTGTKIKALTDGKISQAGAVAGGGGNQITLDEPGGKWYQWYMHMSKIIAKKGQKVSAGDVIGLSGNTGNSTTPHLHIQRMKGYPSNETAVNP

SEQ ID NO: 107 - protein, 104 aa, synthetic construct
RPHMGLDINYPYGSKLYSTLGGTATAKSGYNGGFGNSMWIKSGVMQAIYGHMSELAFSGSKKVKPGDYLGKSGGDPSKQGASAGDSTGAHLHYEMRRNGEPFDP

SEQ ID NO: 108 - protein, 103 aa, synthetic construct
RPHHGLDINYKHDKVYSTMSGTARAFKGWSGGFGNHMEVTNGNVKSIYGHLHKLAFTGSKKVHPGTFLGISGGDPGEDGQGAGSSTGLHLHYEMQWGGVAKDP

SEQ ID NO: 109 - protein, 100 aa, synthetic construct
GVHHGVDYDTPTGTPIRTPMGGRVRSWYDNYGGGKAITVQKGRTFLWFMHLSEQLRRTGEQIKAGQLIGKSGNTGSMTNYRHLHFQVNEGGEANRYSTDP

SEQ ID NO: 110 - protein, 100 aa, synthetic construct
WPHPGIDLPYHHEPIFSTLNGKAYNKFMAGGFGKYILVKSGALEAFYAHLSKSFIKDGQSVHAGQKLGISGGDPGLAISGASTGPHLHYEMHRNGKPINP

SEQ ID NO: 111 - protein, 97 aa, synthetic construct
PFHEGVDFPFVYETIRTPMGGRVARQSFMAGGYGNWVKVISGAMELIFAHLKNFSKTPPSGKNVKPGDVIGLTGNTGFSTGPHLHFGIRKNGRDIDP

SEQ ID NO: 112 - protein, 102 aa, synthetic construct
GRHYGVDFGMPVGTNIYAVKGGIADHVWTDFGGGNSIQIKTGANEWNWYMHLSKQIARQGQRIKAGQLIGLSGATGNFVRGAHLHFQLMRGSHPGNDTAVDP

SEQ ID NO: 113 - protein, 97 aa, synthetic construct
PFHEGVDFPFVYQTVRTPMGGRVARQSFMPGGYGNWVKVISGAMELIFAHLKNFSKTPASGKNVKPGDVIGLTGNTGFSTGPHLHFGIRKNGRDIDP

SEQ ID NO: 114 - protein, 101 aa, synthetic construct
TPHYGVDYASSVGSPIRAARGGVVLSSGWSNYGGGNQIVIYNPKLNKTFTYMHMLSDLRVKKGQQVVAGQLIGRMGNTGNSSGPHLHFQVNEGKGFTAAGT

SEQ ID NO: 115 - protein, 106 aa, synthetic construct
NGHAGIDYGAATGTPIPSPIDGKVIQSWFSPNQPSGGNETQIWDGHKYTHIFMHQSKRKVKTGDRVHQGQIIGLVGDTGNSFGSHLHWQVNKGKGYLNNHPDSINP

SEQ ID NO: 116 - protein, 100 aa, synthetic construct
GRHYGIDYATPVGTTLTAPTSGTISKLSNYGGGTVAKLLSGKFTQFFMHLKDIFKTGKVKQGEKFAHTGNSGHWTTGPHLHYQVEKGHSADITNRNTVDP

SEQ ID NO: 117 - protein, 102 aa, synthetic construct
GRHYGIDFGMPTGTNVYAVKGGIADKVWTDYGGGNSIQIKTGANEWNWYMHLSKQLARQGQRIKAGQLIGKSGATGNFVRGAHLHLQLMQGSHPGNDTAKDP

SEQ ID NO: 118 - protein, 102 aa, synthetic construct
GRHYGIDFGMPTGTNIYAVKGGIADKVWTDYGGGNSIQIKTGANEWNWYMHLSKQLVRQGQRIKAGQLIGKSGATGNFVRGAHLHFQLMQGSHPGNDTAKDP

SEQ ID NO: 119 - protein, 100 aa, synthetic construct
AHPGIDLPYIYEKVQTPLEGKVQTKDTGNVGFGHHVVVKAKPYDAYFGHMSKWSVKNGQHVKPGDVLGISGNTGSSTGPHLHYEMNKHGMGSMTGHSIDP

SEQ ID NO: 120 - protein, 100 aa, synthetic construct
GHHYGIDFGAPFGTTINATNSGQLGELHNFGGGLVARLLTGQFTLFFMHLSKILKHGKVQAGEPIAKTGNSGNWTTGPHLHFQVEKGRHNDITNQNTVNP

SEQ ID NO: 121 - protein, 100 aa, synthetic construct
GRHFGIDYGTPAGTVIKAPTSGTVSTLHNYGGGLVAKLLSGKFTQFFMHLQDILKTGRVKKGDKFAKTGNSGHWTTGAHLHYQVEKGNSSDITNKNTVDP

SEQ ID NO: 122 - protein, 95 aa, synthetic construct
PFHEGLDFDYIYEPVPSTINGRAQVMPFHNGGYGKWVKIVKGALEVIYAHLSKYKVKTGHQVRVGQTVGISGNTGFSTGPHLHYEMRWNGRHRDP

SEQ ID NO: 123 - protein, 102 aa, synthetic construct
GRHYGIDFGMPTGTNIYAVKGGIADKVWTDYGGGNSIQIKTGANEWNWYMHLSKQLARQGQRIKAGQLIGKSGATGNFVRGAHLHFQLMRGSHPGNDTAVDP

SEQ ID NO: 124 - protein, 112 aa, synthetic construct
SNDSSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 125 - protein, 102 aa, synthetic construct
KNPPVPAGYTLDKNNVPYKKEAGNYTVANVKGNNVRDGYSTNSRITGVLPNNATIKYDGAYCINGYRWITYIANSGQRRYIATGEVDKAGNRISSFGKFSTI

SEQ ID NO: 126 - protein, 85 aa, synthetic construct
SRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 127 - protein, 113 aa, synthetic construct
MPFLKSAGYGSNSTSSSNNNGYKTNKYGTLYKSESASFTANTDIITRLTGPFRSMPQSGVLRKGLTIKYDEVMKQDGHVWVGYNTNSGKRVYLPVRTWNESTGELGPLWGTIK

SEQ ID NO: 128 - protein, 106 aa, synthetic construct
PSSKPSADKITWNWKGVFYPNPEKAIRVRKTAGLTGTVVEEDSWLYTKDDWVKFDQVIKKDGYWWIRFKYQREGSSTNNFYCAVCRITDKEQKIKNEKYWGTIEWA

SEQ ID NO: 129 - protein, 108 aa, synthetic construct
SASTPATRPVTGSWKKNQYGTWYKPESATFVNGNQPIVTRIGSPFLNAPVGGNLPAGATIVYDEVCIQAGHIWIGYNAYNGNRVYCPVRTCQGVPPSHVPGVAWGTFK

SEQ ID NO: 130 - protein, 271 aa, synthetic construct
MKTQKQVVDYIKSLEGKGWDWDNYAGWQCFDTANYHAYFAMGMSLAGEGAKDIPYKNDFTGKADIYNNTPEFLAQPGDIVVWTSPTFGGGYGHVASVISATLNTITVIEQNWLGGGISKTEVATRRSHSYDPSMVFIRPKYAKSSTVTESKPTTVKPVTKPTSSASTPATRPVTGSWKKNQYGTWYKPESATFVNGNQPIVTRIGSPFLNAPVGGNLPAGATIVYDEVCIQAGHIWIGYNAYNGNRVYCPVRTCQGVPPSHVPGVAWGTFK

SEQ ID NO: 131 - protein, 265 aa, synthetic construct
MKTQKQVVDYIKSLEGKGWDWDNYAGWQCFDTANYHAYFAMGMSLAGEGAKDIPYKNDFTGKADIYNNTPEFLAQPGDIVVWTSPTFGGGYGHVASVISATLNTITVIEQNWLGGGISKTEVATRRSHSYDPSMVFIRPKYAKSSTVTESKPTTVKPVTKPTSKNPPVPAGYTLDKNNVPYKKEAGNYTVANVKGNNVRDGYSTNSRITGVLPNNATIKYDGAYCINGYRWITYIANSGQRRYIATGEVDKAGNRISSFGKFSTI

SEQ ID NO: 132 - protein, 269 aa, synthetic construct
MKTQKQVVDYIKSLEGKGWDWDNYAGWQCFDTANYHAYFAMGMSLAGEGAKDIPYKNDFTGKADIYNNTPEFLAQPGDIVVWTSPTFGGGYGHVASVISATLNTITVIEQNWLGGGISKTEVATRRSHSYDPSMVFIRPKYAKSSTVTESKPTTVKPVTKPTSPSSKPSADKITWNWKGVFYPNPEKAIRVRKTAGLTGTVVEEDSWLYTKDDWVKFDQVIKKDGYWWIRFKYQREGSSTNNFYCAVCRITDKEQKIKNEKYWGTIEWA

SEQ ID NO: 133 - protein, 276 aa, synthetic construct
MKTQKQVVDYIKSLEGKGWDWDNYAGWQCFDTANYHAYFAMGMSLAGEGAKDIPYKNDFTGKADIYNNTPEFLAQPGDIVVWTSPTFGGGYGHVASVISATLNTITVIEQNWLGGGISKTEVATRRSHSYDPSMVFIRPKYAKSSTVTESKPTTVKPVTKPTSMPFLKSAGYGSNSTSSSNNNGYKTNKYGTLYKSESASFTANTDIITRLTGPFRSMPQSGVLRKGLTIKYDEVMKQDGHVWVGYNTNSGKRVYLPVRTWNESTGELGPLWGTIK

SEQ ID NO: 134 - protein, 248 aa, synthetic construct
MKTQKQVVDYIKSLEGKGWDWDNYAGWQCFDTANYHAYFAMGMSLAGEGAKDIPYKNDFTGKADIYNNTPEFLAQPGDIVVWTSPTFGGGYGHVASVISATLNTITVIEQNWLGGGISKTEVATRRSHSYDPSMVFIRPKYAKSSTVTESKPTTVKPVTKPTSSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 135 - protein, 275 aa, synthetic construct
MKTQKQVVDYIKSLEGKGWDWDNYAGWQCFDTANYHAYFAMGMSLAGEGAKDIPYKNDFTGKADIYNNTPEFLAQPGDIVVWTSPTFGGGYGHVASVISATLNTITVIEQNWLGGGISKTEVATRRSHSYDPSMVFIRPKYAKSSTVTESKPTTVKPVTKPTSSNDSSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 136 - protein, 255 aa, synthetic construct
MDGSYLFEYPIWQRFGRYTGGLNFNGGRHYGIDFGMPSGTNVYAVKGGIADKVWTDYGGGNSIQIKTGANEWNWYMHLSKQLARQGQRIKAGQLIGKSGATGNFVRGAHLHFQLMQGSHPGNDTAKDPEKWLKSLKGSGVRSGSGTSSASTPATRPVTGSWKKNQYGTWYKPESATFVNGNQPIVTRIGSPFLNAPVGGNLPAGATIVYDEVCIQAGHIWIGYNAYNGNRVYCPVRTCQGVPPSHVPGVAWGTFK

SEQ ID NO: 137 - protein, 249 aa, synthetic construct
MDGSYLFEYPIWQRFGRYTGGLNFNGGRHYGIDFGMPSGTNVYAVKGGIADKVWTDYGGGNSIQIKTGANEWNWYMHLSKQLARQGQRIKAGQLIGKSGATGNFVRGAHLHFQLMQGSHPGNDTAKDPEKWLKSLKGSGVRSGSGTSKNPPVPAGYTLDKNNVPYKKEAGNYTVANVKGNNVRDGYSTNSRITGVLPNNATIKYDGAYCINGYRWITYIANSGQRRYIATGEVDKAGNRISSFGKFSTI

SEQ ID NO: 138 - protein, 253 aa, synthetic construct
MDGSYLFEYPIWQRFGRYTGGLNFNGGRHYGIDFGMPSGTNVYAVKGGIADKVWTDYGGGNSIQIKTGANEWNWYMHLSKQLARQGQRIKAGQLIGKSGATGNFVRGAHLHFQLMQGSHPGNDTAKDPEKWLKSLKGSGVRSGSGTSPSSKPSADKITWNWKGVFYPNPEKAIRVRKTAGLTGTVVEEDSWLYTKDDWVKFDQVIKKDGYWWIRFKYQREGSSTNNFYCAVCRITDKEQKIKNEKYWGTIEWA

SEQ ID NO: 139 - protein, 260 aa, synthetic construct
MDGSYLFEYPIWQRFGRYTGGLNFNGGRHYGIDFGMPSGTNVYAVKGGIADKVWTDYGGGNSIQIKTGANEWNWYMHLSKQLARQGQRIKAGQLIGKSGATGNFVRGAHLHFQLMQGSHPGNDTAKDPEKWLKSLKGSGVRSGSGTSMPFLKSAGYGSNSTSSSNNNGYKTNKYGTLYKSESASFTANTDIITRLTGPFRSMPQSGVLRKGLTIKYDEVMKQDGHVWVGYNTNSGKRVYLPVRTWNESTGELGPLWGTIK

SEQ ID NO: 140 - protein, 232 aa, synthetic construct
MDGSYLFEYPIWQRFGRYTGGLNFNGGRHYGIDFGMPSGTNVYAVKGGIADKVWTDYGGGNSIQIKTGANEWNWYMHLSKQLARQGQRIKAGQLIGKSGATGNFVRGAHLHFQLMQGSHPGNDTAKDPEKWLKSLKGSGVRSGSGTSSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 141 - protein, 259 aa, synthetic construct
MDGSYLFEYPIWQRFGRYTGGLNFNGGRHYGIDFGMPSGTNVYAVKGGIADKVWTDYGGGNSIQIKTGANEWNWYMHLSKQLARQGQRIKAGQLIGKSGATGNFVRGAHLHFQLMQGSHPGNDTAKDPEKWLKSLKGSGVRSGSGTSSNDSSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 142 - protein, 467 aa, Twortvirus twort
MKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNMVKRGYNPVGVILHNDAGSMTGLQYKNNLQNAGYNRWAQGIAHSYISEGQVWQALGESRIAWHCANQWGNKNLYGIEICQSMTASDEQFLKNEQTAFYEASRMLKKWGLKPDKNTVRLHMEYYQTACPHRSMKLHVGKDPTKTSITQADIEKLKEYFIKQIKMYYEGKTPVPTVVNQKAKTKPVKQSSTSGWNVNNYGTYYKSESATFKCTARQGIVTRYTGPFTTCPQAGVLYYGQSVTYDTVCKQDGYVWISWTTNGGQDVWMPVRTWDKNTDIMGQLWGDIY

SEQ ID NO: 143 - protein, 166 aa, synthetic construct
MKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDG

SEQ ID NO: 144 - protein, 255 aa, synthetic construct
MKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGTSSRSYRETGTMTVTVDALNVRRAPNTSGEIVAVYKRGESFDYDTVIIDVNGYVWVSYIGGSGKRNYVATGATKDGKRFGNAWGTFKLE

SEQ ID NO: 145 - protein, 277 aa, synthetic construct
MKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGTSKNPPVPAGYTLDKNNVPYKKEAGNYTVANVKGNNVRDGYSTNSRITGVLPNNATIKYDGAYCINGYRWITYIANSGQRRYIATGEVDKAGNRISSFGKFSTIKLAAALE

SEQ ID NO: 146 - protein, 267 aa, synthetic construct
MDGSYLFNYPVWQRFGSYTGGLGFNGGRHYGMDFGMPAGTSVYAVKGGTVDKLWYDYGGGNSIQIKTGPGEWNWYMHLSKQLVKLGEQFKTGQLIAKSGETGAYCKGAHLHFQLMRGDHPGNDTAIDPEPYLKSMKGAAGGNGADVARRAILKTSSNDSSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 147 - protein, 7 aa, synthetic construct
  VARIANT 1: note = replace with F
  VARIANT 4: note = replace with Q
  VARIANT 5: note = S or N
  VARIANT 6: note = K or R
  VARIANT 7: note = Q or R
YMHLXXX

SEQ ID NO: 148 - protein, 9 aa, synthetic construct
  VARIANT 1: note = Y, F or G
  VARIANT 5: note = N or H
  VARIANT 6: note = S, Q or A
  VARIANT 7: note = I or V
  VARIANT 8: note = any amino acid
XGGGXXXXI

SEQ ID NO: 149 - protein, 389 aa, Staphylococcus simulans
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 150 - protein, 362 aa, Staphylococcus capitis
MDTNRKFTLVKSLSIGLGTFLVGSVFLTVNDEASASTKVDAPKVEQEAPAKADAPKVEQEAPAKADAPKVEQEAPAKVDAPKVEQEAPAKVDAPKVEQEAPAKADAPKVEQKRTFVREAAQSNHSASWLNNYKKGYGYGPYPLGINGGNHYGVDFFMNVGTPVRAISDGKIVEAGWTNYGGGNEIGLVENDGVHRQWYMHLSKFNVKVGDRVKAGQIIGWSGSTGYSTAPHLHFQRMTNSFSNNTAQDPMPFLKSAGYGSNSTSSSNNNGYKTNKYGTLYKSESASFTANTDIITRLTGPFRSMPQSGVLRKGLTIKYDEVMKQDGHVWVGYNTNSGKRVYLPVRTWNESTGELGPLWGTIK

SEQ ID NO: 151 - protein, 316 aa, Staphylococcus aureus
MKKLTAAAIATMGFATFTMAHQADAAETTNTQQAHTQMSTQSQDVSYGTYYTIDSNGDYHHTPDGNWNQAMFDNKEYSYTFVDAQGHTHYFYNCYPKNANANGSGQTYVNPATAGDNNDYTASQSQQHINQYGYQSNVGPDASYYSHSNNNQAYNSHDGNGKVNYPNGTSNQNGGSASKATASGHAKDASWLTSRKQLQPYGQYHSGGAHYGVDYAMPENSPVYSLTDGTVVQAGWSNYGGGNQVTIKEANSNNYQWYMHNNRLTVSAGDKVKAGDQIAYSGSTGNSTAPHVHFQRMSGGIGNQYAVDPTSYLQSR

SEQ ID NO: 152 - protein, 154 aa, synthetic construct
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNT

SEQ ID NO: 153 - protein, 146 aa, synthetic construct
MSNHSASWLNNYKKGYGYGPYPLGINGGNHYGVDFFMNVGTPVRAISDGKIVEAGWTNYGGGNEIGLVENDGVHRQWYMHLSKFNVKVGDRVKAGQIIGWSGSTGYSTAPHLHFQRMTNSFSNNTAQDPMPFLKSAGYGSNSTSSS

SEQ ID NO: 154 - protein, 133 aa, synthetic construct
MHAKDASWLTSRKQLQPYGQYHSGGAHYGVDYAMPENSPVYSLTDGTVVQAGWSNYGGGNQVTIKEANSNNYQWYMHNNRLTVSAGDKVKAGDQIAYSGSTGNSTAPHVHFQRMSGGIGNQYAVDPTSYLQSR

SEQ ID NO: 155 - protein, 268 aa, synthetic construct
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTTSSNDSSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 156 - protein, 260 aa, synthetic construct
MSNHSASWLNNYKKGYGYGPYPLGINGGNHYGVDFFMNVGTPVRAISDGKIVEAGWTNYGGGNEIGLVENDGVHRQWYMHLSKFNVKVGDRVKAGQIIGWSGSTGYSTAPHLHFQRMTNSFSNNTAQDPMPFLKSAGYGSNSTSSSTSSNDSSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 157 - protein, 247 aa, synthetic construct
MHAKDASWLTSRKQLQPYGQYHSGGAHYGVDYAMPENSPVYSLTDGTVVQAGWSNYGGGNQVTIKEANSNNYQWYMHNNRLTVSAGDKVKAGDQIAYSGSTGNSTAPHVHFQRMSGGIGNQYAVDPTSYLQSRTSSNDSSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS